(19) United States
(12) Patent Application Publication
(10) Pub. No.: US 2024/0425603 A1
(43) Pub. Date: Dec. 26, 2024

(54) ANTI-VEGFR1 ANTIBODIES AND THEIR USES

(71) Applicant: Janssen Biotech, Inc., Horsham, PA (US)

(72) Inventors: Matthew D. Breyer, Indianapolis, IN (US); Romer A. Gonzalez Villalobos, East Greenwich, RI (US); Jingjun Li, Newton, MA (US); Thomas J. Rutkoski, Philadelphia, PA (US); Ronald V. Swanson, Del Mar, CA (US); Gang Zheng, Chestnut Hill, MA (US); Songmao Zheng, Ambler, PA (US); Xirong Zheng, Malvern, PA (US)

(21) Appl. No.: 18/683,892

(22) PCT Filed: Aug. 12, 2022
(86) PCT No.: PCT/US2022/074891

§ 371 Date: Feb. 15, 2024

Related U.S. Application Data
(60) Provisional application No. 63/233,343, filed on Aug. 16, 2021.
(60) Provisional application No. 63/322,273, filed on Mar. 22, 2022.

(51) Int. Cl. C07K 16/28; A61K 39/00; A61P 13/12

(52) U.S. Cl. CPC C07K 16/2863; A61P 13/12; A61K 2039/505; C07K 2317/34; C07K 2317/52; C07K 2317/565; C07K 2317/76; C07K 2317/92

(57) ABSTRACT

Disclosed herein are antibodies or antigen binding fragments thereof that bind vascular endothelial growth factor receptor 1 (VEGFR1), polynucleotides, vectors, host cells and methods of treating chronic kidney disease using the same.

### CROSS-REFERENCE TO RELATED APPLICATIONS

This application claims priority to U.S. Provisional Application No. 63/233,343 filed on Aug. 16, 2021, and to U.S. Provisional Application No. 63/322,273 filed on Mar. 22, 2022, titled “ANTI-VEGFR1 ANTIBODIES AND THEIR USES”, the disclosure of each of which is incorporated by reference herein in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Jul. 22, 2022, is named JBI6512WOPCT1_SL.xml and is 181,681 bytes in size.

### TECHNICAL FIELD

Provided are proteins comprising antigen binding domains or fragments thereof that bind vascular endothelial growth factor receptor 1 (VEGFR1), polynucleotides, vectors, host cells and methods of treating chronic kidney disease using the same.

### BACKGROUND

Chronic kidney disease (CKD) is a worldwide public health problem that continues to grow with the aging population and the diabetes/obesity epidemic. CKD increases the risk for all-cause and cardiovascular mortality as well as end stage kidney disease, which requires dialysis or kidney transplant for survival. CKD stage 4-5 patients are at highest risk for kidney failure with 5 year mortality rate ˜40%. Diabetes (38%) and hypertension (25%) are the major causes of late stage and end stage kidney disease. Diabetic kidney disease, the most common CKD, occurs in ˜25% of the patients with type 2 diabetes within 10 years of diagnosis. Despite the standard of care renin-angiotensin-aldosterone system inhibitors i.e., angiotensin converting enzyme inhibitors or angiotensin receptor blockers, or recent introduction of SGLT2 inhibitors, most “at-risk” patients or poor responders still progress to end stage kidney disease. In the US alone over 100,000 patients per year progress to dialysis. 70% of patients on dialysis die within 5 years.

The medical need for therapies that prevent or treat end stage kidney disease by renal protection or functional restoration in late-stage CKD patients remains extraordinarily high.

In the kidney, vascular endothelial growth factor A (VEGFA) made by glomerular podocytes and tubular epithelial cells is essential to maintain glomerular integrity, renal microvasculature, and function. In CKD patients, mRNA microarray analyses of patient kidney biopsies showed decreased VEGFA expression and the level of VGFA expression in glomeruli and tubulointerstitium is correlated with eGFR, proteinuria or vascular rarefaction (Bortoloso, Del Prete et al. 2004, Martini, Nair et al. 2014, Pan, Jiang et al. 2018). In healthy kidney, locally generated VEGFA, signaling through VEGFR2 promotes endothelial health protecting glomerular filtration barrier. In CKD, VEGFA deficiency together with sequestration by its decoy receptor VEGFR1 results in suppressed VEGFR2 signaling. Anti-VEGFR1 blocking antibodies were developed to block VEGFA sequestration, increase local VEGFA availability, and enable endothelial protection, preservation of glomerular filtration barrier and renal function restoration.

### BRIEF SUMMARY

Provided herein is an isolated antibody or antigen binding fragment thereof that binds to an epitope within SEQ ID NO: 173 of VEGFR1 and prevents the binding of VEGFA to VEGFR1.

In some embodiments, the antibody or antigen binding fragment thereof that binds VEGFR1 binds to an epitope on VEGFR1 having the amino acid sequence FPLDTL (SEQ ID NO: 143) or EIGL (SEQ ID NO: 144). In some embodiments, the antibody or antigen binding fragment thereof that binds VEGFR1 binds to an epitope on VEGFR1 having the amino acid sequence FPLDTL (SEQ ID NO: 143) and EIGL (SEQ ID NO: 144).

In some embodiments, the isolated antibody or antigen binding fragment thereof binds human, mouse, rat and/or cynomolgus monkey VEGFR1.

In some embodiments, the isolated antibody or antigen binding fragment thereof binds human VEGFR1. In some embodiments, the isolated antibody or antigen binding fragment thereof binds human VEGFR1 and a VEGFR1 from at least one species selected from the group consisting of cynomolgus monkey, mouse and rat. In some embodiments, the isolated antibody or antigen binding fragment thereof binds human VEGFR1 and a VEGFR1 from at least two species selected from the group consisting of cynomolgus monkey, mouse and rat. In some embodiments, the isolated antibody or antigen binding fragment thereof binds human, cynomolgus monkey, mouse and rat VEGFR1.

In some embodiments, the antibody or antigen binding fragment thereof binds to human VEGFR1, mouse VEGFR1, and cynomolgus monkey VEGFR1, with a KD of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

In some embodiments, an isolated antibody or antigen binding fragment thereof of the application comprises a heavy chain variable region (VH) having the heavy chain complementarity determining regions (HCDRs) of a VH comprising the amino acid sequence of SEQ ID NO: 31, 33, 34, 36, 38 or 39, and a light chain variable region (VL) having the light chain complementarity determining regions (LCDRs) of a VL comprising the amino acid sequence of SEQ ID NO: 32, 35, 37 or 40.

In certain embodiments, the isolated antibody or antigen binding fragment thereof comprises:
a. the HCDRs of the VH having the amino acid sequence of SEQ ID NO: 31, and the LCDRs of the VL having the amino acid sequence of SEQ ID NO: 32;
b. the HCDRs of the VH having the amino acid sequence of SEQ ID NO: 33, and the LCDRs of the VL having the amino acid sequence of SEQ ID NO: 32.
c. the HCDRs of the VH having the amino acid sequence of SEQ ID NO: 34, and the LCDRs of the VL having the amino acid sequence of SEQ ID NO: 35;
d. the HCDRs of the VH having the amino acid sequence of SEQ ID NO: 36, and the LCDRs of the VL having the amino acid sequence of SEQ ID NO: 37;
e. the HCDRs of the VH having the amino acid sequence of SEQ ID NO: 38, and the LCDRs of the VL having the amino acid sequence of SEQ ID NO: 37; or
f. the HCDRs of the VH having the amino acid sequence of SEQ ID NO: 39, and the LCDRs of the VL having the amino acid sequence of SEQ ID NO: 40.

Provided herein is an isolated antibody or antigen binding fragment thereof comprising the HCDR1, the HCDR2, the HCDR3, the LCDR1, the LCDR2 and the LCDR3 of
SEQ ID NOs: 7, 175, 9, 10, 11 and 12, respectively;
SEQ ID NOs: 7, 8, 9, 10, 11 and 12, respectively;
SEQ ID NOs: 13, 14, 15, 16, 17 and 18, respectively;
SEQ ID NOs: 19, 20, 21, 22, 23 and 24, respectively;
SEQ ID NOs: 25, 26, 27, 28, 29, and 30, respectively;
SEQ ID NOs: 71, 176, 73, 74, 75 and 76, respectively;
SEQ ID NOs: 71, 72, 73, 74, 75 and 76, respectively;
SEQ ID NOs: 77, 78, 79, 80, 81, and 82, respectively;
SEQ ID NOs: 83, 84, 85, 86, 87, and 88, respectively;
SEQ ID NOs: 89, 90, 91, 92, 93 and 94, respectively;
SEQ ID NOs: 95, 177, 97, 98, 99 and 100, respectively;
SEQ ID NOs: 95, 96, 97, 98, 99 and 100, respectively;
SEQ ID NOs: 101, 102, 103, 104, 105 and 106, respectively;
SEQ ID NOs: 107, 108, 109, 110, 111, and 112, respectively;
SEQ ID NOs: 113, 114, 115, 116, 117, and 118, respectively;
SEQ ID NOs: 119, 178, 121, 122, amino acid sequence LNS, and SEQ ID NO: 124, respectively;
SEQ ID NOs: 119, 120, 121, 122, amino acid sequence LNS, and SEQ ID NO: 124, respectively;
SEQ ID NOs: 125, 126, 127, 128, amino acid sequence FNF, and SEQ ID NO: 130, respectively;
SEQ ID NOs: 131, 132, 133, 134, amino acid sequence YD, and SEQ ID NO: 136, respectively; or
SEQ ID NOs: 137, 138, 139, 140, amino acid sequence FNS, and SEQ ID NO: 142, respectively;
   wherein the antibody or antigen binding fragment thereof binds VEGFR1.

Also disclosed is an isolated antibody or antigen binding fragment thereof that binds VEGFR1 comprising:
the VH of SEQ ID NO: 31 and the VL of SEQ ID NO: 32;
the VH of SEQ ID NO: 33 and the VL of SEQ ID NO: 32;
the VH of SEQ ID NO: 34 and the VL of SEQ ID NO: 35;
the VH of SEQ ID NO: 36 and the VL of SEQ ID NO: 37;
the VH of SEQ ID NO: 38 and the VL of SEQ ID NO: 37; or
the VH of SEQ ID NO: 39 and the VL of SEQ ID NO: 40.

Also disclosed is an isolated antibody or antigen binding fragment thereof comprising a VH which is at least 80% (e.g. at least 85%, at least 90%, at least 95%, at least 99% or 100%) identical to the VH of SEQ ID NO: 39 and a VL which is at least 80% (e.g. at least 85%, at least 90%, at least 95%, at least 99% or 100%) identical to the VL of SEQ ID NO: 40.

Also disclosed is an isolated antibody or antigen binding fragment thereof comprising a VH which is at least 80% (e.g. at least 85%, at least 90%, at least 95%, at least 99% or 100%) identical to the VH of SEQ ID NO: 31 and a VL which is at least 80% (e.g. at least 85%, at least 90%, at least 95%, at least 99% or 100%) identical to the VL of SEQ ID NO: 32.

disclosed is an isolated antibody or antigen binding fragment thereof comprising a VH which is at least 80% (e.g. at least 85%, at least 90%, at least 95%, at least 99% or 100%) identical to the VH of SEQ ID NO: 33 and a VL which is at least 80% (e.g. at least 85%, at least 90%, at least 95%, at least 99% or 100%) identical to the VL of SEQ ID NO: 32.

Also disclosed is an isolated antibody or antigen binding fragment thereof comprising a VH which is at least 80% (e.g. at least 85%, at least 90%, at least 95%, at least 99% or 100%) identical to the VH of SEQ ID NO: 34 and a VL which is at least 80% (e.g. at least 85%, at least 90%, at least 95%, at least 99% or 100%) identical to the VL of SEQ ID NO: 35.

Also disclosed is an isolated antibody or antigen binding fragment thereof comprising a VH which is at least 80% (e.g. at least 85%, at least 90%, at least 95%, at least 99% or 100%) identical to the VH of SEQ ID NO: 36 and a VL which is at least 80% (e.g. at least 85%, at least 90%, at least 95%, at least 99% or 100%) identical to the VL of SEQ ID NO: 37.

Also disclosed is an isolated antibody or antigen binding fragment thereof comprising a VH which is at least 80% (e.g. at least 85%, at least 90%, at least 95%, at least 99% or 100%) identical to the VH of SEQ ID NO: 38 and a VL which is at least 80% (e.g. at least 85%, at least 90%, at least 95%, at least 99% or 100%) identical to the VL of SEQ ID NO: 37.

The disclosure also provides an isolated antibody or antigen binding fragment thereof that binds VEGFR1, comprising an amino acid sequence selected from the group consisting of SEQ ID NOs: 51, 52, 53, 54, 55, 56, 57, 58, 59 and 60.

The disclosure also provides an isolated antibody or antigen binding fragment thereof that binds VEGFR1 comprising an amino acid sequence at least 80% (e.g. at least 85%, at least 90%, at least 95%, at least 99% or 100%) identical to the amino acid sequence of SEQ ID NO: 51 or 52.

The disclosure also provides an isolated antibody or antigen binding fragment thereof that binds VEGFR1 comprising an amino acid sequence at least 80% (e.g. at least 85%, at least 90%, at least 95%, at least 99% or 100%) identical to the amino acid sequence of SEQ ID NO: 53 or 52.

The disclosure also provides an isolated antibody or antigen binding fragment thereof that binds VEGFR1 comprising an amino acid sequence at least 80% (e.g. at least 85%, at least 90%, at least 95%, at least 99% or 100%) identical to the amino acid sequence of SEQ ID NO: 54 or 55.

The disclosure also provides an isolated antibody or antigen binding fragment thereof that binds VEGFR1 comprising an amino acid sequence at least 80% (e.g. at least 85%, at least 90%, at least 95%, at least 99% or 100%) identical to the amino acid sequence of SEQ ID NO: 56 or 57.

The disclosure also provides an isolated antibody or antigen binding fragment thereof that binds VEGFR1 comprising an amino acid sequence at least 80% (e.g. at least 85%, at least 90%, at least 95%, at least 99% or 100%) identical to the amino acid sequence of SEQ ID NO: 58 or 57.

The disclosure also provides an isolated antibody or antigen binding fragment thereof that binds VEGFR1 comprising an amino acid sequence at least 80% (e.g. at least 85%, at least 90%, at least 95%, at least 99% or 100%) identical to the amino acid sequence of SEQ ID NO: 59 or 60.

In some embodiments, an isolated antibody of the application comprises:
a. a HC comprising the amino acid sequence of SEQ ID NO: 51 and a LC comprising the amino acid sequence of SEQ ID NO: 52;
b. a HC comprising the amino acid sequence of SEQ ID NO: 53 and a LC comprising the amino acid sequence of SEQ ID NO: 52;
c. a HC comprising the amino acid sequence of SEQ ID NO: 54 and a LC comprising the amino acid sequence of SEQ ID NO: 55;
d. a HC comprising the amino acid sequence of SEQ ID NO: 56 and a LC comprising the amino acid sequence of SEQ ID NO: 57;
e. a HC comprising the amino acid sequence of SEQ ID NO: 58 and a LC comprising the amino acid sequence of SEQ ID NO: 57; or
f. a HC comprising the amino acid sequence of SEQ ID NO: 59 and a LC comprising the amino acid sequence of SEQ ID NO: 60.

Also disclosed is an isolated antibody or antigen binding fragment thereof comprising
a HCDR1, a HCDR2, a HCDR3, a LCDR1, a LCDR2 and a LCDR3 of SEQ ID NOs: 7, 175, 9, 10, 11, and 12, respectively;
a VH of SEQ ID NO: 31 and a VL of SEQ ID NO: 32; and/or
a HC of SEQ ID NO: 51 and a LC of SEQ ID NO: 52;
and wherein the antibody or antigen binding fragment thereof binds VEGFR1.

Also disclosed is an isolated antibody or antigen binding fragment thereof comprising
a HCDR1, a HCDR2, a HCDR3, a LCDR1, a LCDR2 and a LCDR3 of SEQ ID NOs: 7, 8, 9, 10, 11, and 12, respectively;
a VH of SEQ ID NO: 33 and a VL of SEQ ID NO: 32; and/or
a HC of SEQ ID NO: 53 and a LC of SEQ ID NO: 52;
and wherein the antibody or antigen binding fragment thereof binds VEGFR1.

Also disclosed is an isolated antibody or antigen binding fragment thereof comprising
a HCDR1, a HCDR2, a HCDR3, a LCDR1, a LCDR2 and a LCDR3 of SEQ ID NOs: 13, 14, 15, 16, 17 and 18, respectively;
a VH of SEQ ID NO: 34 and a VL of SEQ ID NO: 35; and/or
a HC of SEQ ID NO: 54 and a LC of SEQ ID NO: 55;
and wherein the antibody or antigen binding fragment thereof binds VEGFR1.

Also disclosed is an isolated antibody or antigen binding fragment thereof comprising
a HCDR1, a HCDR2, a HCDR3, a LCDR1, a LCDR2 and a LCDR3 of SEQ ID NOs: 19, 20, 21, 22, 23, and 24, respectively;
a VH of SEQ ID NO: 36 and a VL of SEQ ID NO: 37; and/or
a HC of SEQ ID NO: 56 and a LC of SEQ ID NO: 57;
and wherein the antibody or antigen binding fragment thereof binds VEGFR1.

Also disclosed is an isolated antibody or antigen binding fragment thereof comprising
a HCDR1, a HCDR2, a HCDR3, a LCDR1, a LCDR2 and a LCDR3 of SEQ ID NOs: 19, 20, 21, 22, 23, and 24, respectively;
a VH of SEQ ID NO: 38 and a VL of SEQ ID NO: 37; and/or
a HC of SEQ ID NO: 58 and a LC of SEQ ID NO: 57;
and wherein the antibody or antigen binding fragment thereof binds VEGFR1.

Also disclosed is an isolated antibody or antigen binding fragment thereof comprising
a HCDR1, a HCDR2, a HCDR3, a LCDR1, a LCDR2 and a LCDR3 of SEQ ID NOs: 25, 26, 27, 28, 29, and 30, respectively;
a VH of SEQ ID NO: 39 and a VL of SEQ ID NO: 40; and/or
a HC of SEQ ID NO: 59 and a LC of SEQ ID NO: 60;
and wherein the antibody or antigen binding fragment thereof binds VEGFR1.

In some embodiments, the isolated antibody or antigen binding fragment thereof is conjugated to a half-life extending moiety. Optionally, the half-life extending moiety is an immunoglobulin (Ig), a fragment of the Ig, an Ig constant region, a fragment of the Ig constant region, a Fc region, transferrin, albumin, an albumin binding domain or polyethylene glycol. Optionally, the fragment of the Ig constant region comprises a Fc region. Optionally, the antibody or antigen binding fragment that binds VEGFR1 is conjugated to the C-terminus of the Ig constant region or the fragment of the Ig constant region.

Optionally, the Ig constant region or the fragment of the Ig constant region is an IgG1, an IgG2, an IgG3 or an IgG4 isotype. Optionally, the Ig constant region or the fragment of the Ig constant region is an IgG1 isotype. Optionally, the Ig constant region or the fragment of the Ig constant region is an IgG4 isotype. Optionally, the Ig constant region or the fragment of the Ig constant region comprises at least one mutation that results in reduced binding of the protein to a Fcγ receptor (FcγR). Optionally, the at least one mutation that results in reduced binding of the protein to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. Optionally, the mutations that results in reduced binding of the protein to the FcγR are L234A_L235A_D265S. Optionally, the Ig constant region or the fragment of the Ig constant region comprises at least one mutation that modulates a half-life of the protein. Optionally the at least one mutation that modulates the half-life of the protein is selected from the group consisting of H435A, P257I/N434H, D376V/N434H, M252Y/S254T/T256E/H433K/N434F, T308P/N434A and H435R, wherein residue numbering is according to the EU index.

The disclosure also provides an immunoconjugate comprising the isolated antibody or antigen binding fragment thereof conjugated to a therapeutic agent or an imaging agent.

The disclosure also provides a pharmaceutical composition comprising the isolated antibody or antigen binding fragment thereof that binds VEGFR1 and a pharmaceutically acceptable carrier.

The disclosure also provides a polynucleotide encoding the isolated antibody or antigen binding fragment thereof that binds VEGFR1.

Optionally, the polynucleotide encoding the isolated antibody or antigen binding fragment thereof that binds VEGFR1 comprises a polynucleotide sequence of SEQ ID NOs: 41, 42, 43, 44, 45, 46, 47, 48, 49 50, 61, 62, 63, 74, 65, 66, 67, 68, 69, or 70.

Optionally, the polynucleotide encoding the isolated antibody or antigen binding fragment thereof that binds VEGFR1 comprises a polynucleotide which is at least 80% (e.g. at least 85%, at least 90%, at least 95%, at least 99% or 100%) identical to the polynucleotide sequence of SEQ ID NOs: 41, 42, 43, 44, 45, 46, 47, 48, 49, 50, 61, 62, 63, 64, 65, 66, 67, 68, 69, or 70.

The disclosure also provides a vector comprising the polynucleotide of the disclosure.

The disclosure also provides a host cell comprising the polynucleotide or vector of the disclosure.

The disclosure also provides a host cell expressing the isolated antibody or antigen binding fragment thereof and/or comprising the polynucleotide or vector of the disclosure.

The disclosure also provides a method of preventing the binding of VEGFA to a VEGFR1 in a subject in need thereof, comprising administering to the subject an effective amount of any of the isolated antibodies or antigen binding fragments thereof of the disclosure, any of the immunoconjugates of the disclosure, any of the pharmaceutical compositions of the disclosure, any of the isolated polynucleotides or vectors of the disclosure, or any of the host cells of the disclosure, to the subject to thereby prevent the binding of VEGFA to the VEGFR1.

The disclosure also provides a method of treating and slowing the progression of a chronic kidney disease (CKD) in a subject, comprising administering a therapeutically effective amount of any of the isolated antibody or antigen binding fragment thereof of the disclosure, any of the immunoconjugate of the disclosure, or any of the pharmaceutical composition of the disclosure, any of the isolated polynucleotide or vector of the disclosure, or any of the host cell of the disclosure, to the subject for a time sufficient to treat the CKD. Optionally, the subject has advanced stage 4 or 5 chronic kidney disease. Optionally, the subject has early-stage chronic kidney disease.

The disclosure also provides a kit comprising any of the isolated antibody or antigen binding fragment thereof of the disclosure, any of the immunoconjugate of the disclosure, any of the pharmaceutical composition of the disclosure, any of the isolated polynucleotide or vector of the disclosure, or any of the host cell of the disclosure.

The disclosure also provides a method of treating CKD in a subject in need thereof, comprising administering to the subject a therapeutically effective amount of any of the isolated antibody or antigen binding fragment thereof of the disclosure, any of the immunoconjugate of the disclosure, any of the pharmaceutical composition of the disclosure, any of the isolated polynucleotide or vector of the disclosure, or any of the host cell of the disclosure, to the subject for a time sufficient to decrease proteinuria in the subject.

The disclosure also provides a method of decreasing proteinuria in a subject in need thereof, comprising administering to the subject a therapeutically effective amount of any of the isolated antibody or antigen binding fragment thereof of the disclosure, any of the immunoconjugate of the disclosure, any of the pharmaceutical composition of the disclosure, any of the isolated polynucleotide or vector of the disclosure, or any of the host cell of the disclosure, to the subject for a time sufficient to decrease proteinuria in the subject.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows the amino acid sequence alignment of the ligand-binding region of VEGFR1 (Domains 2-3) from various species. Residues identical to human sequence are indicated with a dot. Non-identical residues are shown explicitly. FIG. 1 discloses SEQ ID NOS 179-184, respectively, in order of appearance.

FIG. 2 shows a presentative FACS binding data depicting the species cross-reactivity of VEGFR1 antibodies (VGFB54, VGFB71, VGFB78 and VGFB82) to human, cynomolgus, and mouse VEGFR1 but retaining specificity for VEGFR1 over VEGFR2 or VEGFR3. Human VEGR1, cyno VEGFR1, murine VEGFR1, human VEGFR2, and human VEGFR3 were over-expressed on the surface of cells. Mean fluorescence intensity is plotted using cell lines over-expressing the indicated VEGFR proteins.

FIG. 3 shows the assessment of the relative frequency of the SHM Leucine (L) compared to Glutamine (Q) at position 105 of VGFB78 VH.

FIG. 4A shows the paratope map of the heavy chain variable domain of four anti-VEGFR1 antibody (VGFB54, VGFB71, VGFB78 and VGFB82) against VEGFR1 (VGFW1). FIG. 4A discloses SEQ ID NOs: 31, 34, 36, and 39, respectively, in order of appearance.

FIG. 4B shows the paratope map of the light chain variable domain of four anti-VEGFR1 antibody light chain (VGFB54, VGFB71, VGFB78 and VGFB82) against VEGFR1 (VGFW1). Highlighted residues represent the HCDRs and LCDRs based on the IMTG delineation. The residues with free energy change-1.0 kcal/mol or less upon binding to VEGFR1 are shown in gradient color from medium gray to dark gray as shown in the scale. The residues which were not stabilized upon binding to VEGFR1 were shown in light gray. The residues which exchange too fast or too slow to see the perturbation upon the binding are shown gray. The residues without color indicate the HDX behaviors were not monitored, because there is no peptide to cover the residues or the residues are the first two residues of a peptide. FIG. 4B discloses SEQ ID NOs 32, 35, 37, and 40, respectively, in order of appearance.

FIG. 5 shows the plasma concentration (ng/mL) of total and free VGFB54 over time following a single IV/SC administration in cynomolgus monkeys.

FIG. 6 shows the plasma concentration (ng/ml) of total and free VGFB78 over time following a single IV/SC administration in cynomolgus monkeys.

FIG. 7 shows the Plasma concentration (ng/ml) of total and free VGFB82 over time following a single IV/SC administration in cynomolgus monkeys

FIG. 8 shows the plasma concentration (ng/ml) of total and free VGFB80 over time following a single IV/SC administration in cynomolgus monkeys

FIG. 9 shows the total PlGF plasma concentration (pg/mL) over time following a single IV/SC administration of VGFB54 in cynomolgus monkeys

FIG. 10 shows the total PlGF plasma concentration (pg/mL) over time following a single IV/SC administration of VGFB78 in cynomolgus monkeys

FIG. 11 shows the total PlGF plasma concentration (pg/mL) over time following a single IV/SC administration of VGFB82 in cynomolgus monkeys.

FIG. 12 shows the total PlGF plasma concentration (pg/mL) over time following a single IV/SC administration of VGFB80 in cynomolgus monkeys

### DETAILED DESCRIPTION OF ILLUSTRATIVE EMBODIMENTS

### Definitions

The disclosed isolated anti-VEGFR1 antibody, antigen binding fragment thereof, polynucleotides, vectors, cells, compositions, kits, and methods may be understood more readily by reference to the following detailed description taken in connection with the accompanying figures, which form a part of this disclosure. It is to be understood that the disclosed antibodies antigen binding domains, antibody fragments, polynucleotides, vectors, cells, compositions, kits, and methods are not limited to those specifically described and/or shown herein, and that the terminology used herein is for the purpose of describing particular embodiments by way of example only and is not intended to be limiting of the claimed antibody, antigen binding domains, antibody fragments, polynucleotides, vectors, cells, compositions, kits, and methods.

Unless specifically stated otherwise, any description as to a possible mechanism or mode of action or reason for improvement is meant to be illustrative only, and the disclosed antibodies, antigen binding domains, antibody fragments, polynucleotides, vectors, cells, compositions, kits, and methods are not to be constrained by the correctness or incorrectness of any such suggested mechanism or mode of action or reason for improvement.

Throughout this text, the descriptions refer to antibodies, antigen binding fragments thereof and methods of using said antigen binding domains. Where the disclosure describes or claims a feature or embodiment associated with an antigen binding domain, such a feature or embodiment is equally applicable to the methods of using said antigen binding domains. Likewise, where the disclosure describes or claims a feature or embodiment associated with a method of using an antigen binding domain, such a feature or embodiment is equally applicable to the antigen binding domain.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment, combinable with others.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of.” Embodiments described in terms of the phrase “consisting essentially of” (or its equivalents) also provide as embodiments those independently described in terms of “consisting of.”

As used in this specification and the appended claims, the phrase “and fragments thereof” when appended to a list includes fragments of one or more members of the associated list. The list may comprise a Markush group so that, as an example, the phrase “the group consisting of peptides A, B, and C, and fragments thereof” specifies or recites a Markush group including A, B, C, fragments of A, fragments of B, and/or fragments of C.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than 50 amino acids may be referred to as “peptides”.

As used herein, “Tagg” refers to the temperature at which the protein starts to aggregate either through dimerization or oligomerization. The aggregation temperature detects the onset of aggregation, the temperature at which a protein will show a tendency to aggregate. Tagg can be determined by differential scanning calorimetry (DSC), Differential Scanning Fluorimetry (DSF) or by circular dichroism (CD). These techniques can detect small changes in the conformation of the protein and therefore detect the starting point of aggregation. Tagg values can be lower or higher than Tm. In cases where Tagg is lower than Tm, the protein either dimerizes and/or oligomerizes first and then starts unfolding later at higher temperatures than the Tagg. In cases where Tagg is higher than Tm, the protein starts to unfold first and then aggregates at a higher temperature than the Tm. Both events are commonly observed and depend on amino acid composition and protein conformation.

As used herein, “Tm” or “mid-point temperature” “is the temperature midpoint of a thermal unfolding curve. It refers to the temperature where 50% of the amino acid sequence is in its native conformation and the other 50% is denatured. A thermal unfolding curve is typically plotted as a function of temperature. Tm is used to measure protein stability. In general, a higher Tm is an indication of a more stable protein. The Tm can be readily determined using methods well known to those skilled in the art such as Circular Dichroism Spectroscopy, Differential Scanning calorimetry, Differential Scanning Fluorimetry (both intrinsic and extrinsic dye based), UV spectroscopy, FT-IR and Isothermal calorimetry (ITC).

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132:211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77) and AbM (Martin and Thornton J Bmol Biol 263:800-15, 1996). The correspondence between the various delineations and variable region numbering is described (see e.g., Lefranc et al. (2003) Dev Comp Immunol 27:55-77; Honegger and Pluckthun (2001), J Mol Biol 309:657-70; International ImMunoGeneTics (IMGT) database; World Wide Web: imgt.org). Available programs such as abYsis by UCL Business PLC may be used to delineate CDRs. The terms “CDR”, “HCDR1”, “HCDR2”, “HCDR3”, “LCDR1”, “LCDR2” and “LCDR3” as used herein include CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat, supra; Chothia, supra; Martin, supra; Lefranc et al., supra).

TABLE 1

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

The term “variable region” or “variable domain” refers to the heavy or light chain domain that is involved in the binding of the antibody to the antigen. The variable domains of the heavy or light chain (VH and VL, respectively) comprise four framework regions (FR) and three complementarity determining regions (CDRs).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

The terms “kit” and “article of manufacture” are used as synonyms.

All publications, including but not limited to patents and patent applications, cited in this specification are herein incorporated by reference as though fully set forth.

Antibodies that Bind VEGFR1

The disclosure relates to isolated anti-VEGFR1 antibodies and antigen binding fragments thereof. The term “isolated antibody”, “antigen binding fragment thereof” and “anti-VEGFR1 antibody” and the like are used interchangeably and refer to an antibody that binds VEGFR1 and that comprises at least one binding domain specifically binding VEGFR1.

The antibodies of the disclosure possess one or more functional properties, included but not limited to high affinity binding to VEGFR1, blocking VEGFA binding to VEGFR1 and the ability to treat chronic kidney disease (CKD). In some embodiments, the invention relates to isolated antibodies or antigen binding fragments thereof that specifically bind VEGFR1.

As used herein the term “Antibody” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. The term antibody includes full length antibodies, whole antibodies, intact antibodies, antibody fragments, antigen binding fragment and antigen binding domains.

In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to some embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to VEGFR1 is substantially free of antibodies that do not bind to VEGFR1). In addition, an isolated antibody is substantially free of other cellular material and/or chemicals. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, “VEGFR1” or “VEGFR-1” refers to a known protein which is called Vascular Endothelial Growth Factor Receptor 1. VEGFR1 is encoded by the FLT1 gene. Unless specified, as used herein, VEGFR1 refers to human VEGFR1. The amino acid sequence of human VEGFR1 is retrievable from Uniprot (Accession #P17948). The amino acid sequence of full length human VEGFR1 is shown in SEQ ID NO: 170. The sequence of VEGFR1 consists of an extracellular ligand-binding domain (amino acid residues 27-758) comprising seven immunoglobulin (Ig)-like motifs (domain D1-domain D7: D1 residue 32-123; D2 residue 151-214, D3 residue 230-327, D4 residue 335-421, D5 residue 428-553, D6 residue 556-654, D7 residue 661-747), a single transmembrane domain and a cytoplasmic domain comprising a kinase domain split by a kinase insert (amino acid residues 827-1158), and a carboxyl terminus. The amino acid sequence of human VEGFR1 D2 domain is shown in SEQ ID NO: 171. The amino acid sequence of human VEGFR1 D3 domain is shown in SEQ ID NO: 172. The amino acid sequence of human VEGFR1 D2-D3 domain is shown in SEQ ID NO: 173. VEGFR1 is produced both as a membrane receptor and as a soluble protein. Soluble VEGFR1 acts as a negative regulator of the activity of VEGFA by binding this factor and preventing its interaction with its membrane receptor.

human VEGFR1 (Uniprot Accession# P17948)
SEQ ID NO: 170
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKMASTLVVADSRISGIYICIASNKVGTVGRNISF
YITDVPNGFHVNLEKMPTEGEDLKLSCTVNKFLYRDVTWILLRTVNNRTM
HYSISKQKMAITKEHSITLNLTIMNVSLQDSGTYACRARNVYTGEEILQK
KEITIRDQEAPYLLRNLSDHTVAISSSTTLDCHANGVPEPQITWFKNNHK
IQQEPGIILGPGSSTLFIERVTEEDEGVYHCKATNQKGSVESSAYLTVQG
TSDKSNLELITLTCTCVAATLFWLLLTLFIRKMKRSSSEIKTDYLSIIMD
PDEVPLDEQCERLPYDASKWEFARERLKLGKSLGRGAFGKVVQASAFGIK
KSPTCRTVAVKMLKEGATASEYKALMTELKILTHIGHHLNVVNLLGACTK
QGGPLMVIVEYCKYGNLSNYLKSKRDLFFLNKDAALHMEPKKEKMEPGLE
QGKKPRLDSVTSSESFASSGFQEDKSLSDVEEEEDSDGFYKEPITMEDLI
SYSFQVARGMEFLSSRKCIHRDLAARNILLSENNVVKICDFGLARDIYKN
PDYVRKGDTRLPLKWMAPESIFDKIYSTKSDVWSYGVLLWEIFSLGGSPY
PGVQMDEDFCSRLREGMRMRAPEYSTPEIYQIMLDCWHRDPKERPRFAEL
VEKLGDLLQANVQQDGKDYIPINAILTGNSGFTYSTPAFSEDFFKESISA
PKFNSGSSDDVRYVNAFKFMSLERIKTFEELLPNATSMFDDYQGDSSTLL
ASPMLKRFTWTDSKPKASLKIDLRVTSKSKESGLSDVSRPSFCHSSCGHV
SEGKRRFTYDHAELERKIACCSPPPDYNSVVLYSTPPI,
(Ig-like D2 domain)
SEQ ID NO: 171
GRELVIPCRVTSPNITVTLKKFPLDTLIPDGKRIIWDSRKGFIISNATYK
EIGLLTCEATVNGH
(Ig-like D3 domain)
SEQ ID NO: 172
IDVQISTPRPVKLLRGHTLVLNCTATTPLNTRVQMTWSYPDEKNKRASVR
RRIDQSNSHANIFYSVLTIDKMQNKDKGLYTCRVRSGPSFKSVNTSVH
(Ig-like D2-D3)
SEQ ID NO: 173
GRELVIPCRVTSPNITVTLKKFPLDTLIPDGKRIIWDSRKGFIISNATYK
EIGLLTCEATVNGHIDVQISTPRPVKLLRGHTLVLNCTATTPLNTRVQMT
WSYPDEKNKRASVRRRIDQSNSHANIFYSVLTIDKMQNKDKGLYTCRVRS
GPSFKSVNTSVH

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein). The term “K_{D}” refers to the dissociation constant, which is obtained from the ratio of Kd to Ka (i.e., Kd/Ka) and is expressed as a molar concentration (M). K_{D} values for antibodies can be determined using methods in the art in view of the present disclosure. For example, the K_{D} of an antibody can be determined by using surface plasmon resonance (SPR), such as by using a biosensor system, e.g., a Biacore® system, or by using bio-layer interferometry technology, such as an Octet RED96 system. The smaller the value of the K_{D} of an antibody, the higher affinity that the antibody binds to a target antigen.

As used herein, an antibody that “binds to VEGFR1” or that “specifically binds to VEGFR1” refers to an antibody that binds to VEGFR1, preferably human VEGFR1, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less.

In some embodiments, an antibody or antigen binding fragment thereof that “binds to VEGFR1” or that “specifically binds to VEGFR1” refers to an antibody or antigen binding fragment thereof that binds to a VEGFR1 with a K_{D} of 1×10⁻⁷ M or less. In certain embodiments, the anti-VEGFR1 antibody or antigen binding fragment thereof binds specifically to a human VEGFR1, with a K_{D} preferably of 1×10⁻⁸ M or less, more preferably 5×10⁻⁹ M or less, 1×10⁻⁹ M or less, 5×10⁻¹⁰ M or less, or 1×10⁻¹⁰ M or less.

The anti-VEGFR1 antibody of the disclosure include whole antibodies, antibody fragments that specifically bind to VEGFR1, and antigen binding fragments thereof that specifically binds to VEGFR1.

In some embodiments, the anti-VEGFR1 antibody of the disclosure include whole antibodies or full-length antibodies, Fv fragments, single chain scFv fragments (scFv), Fab, F (ab) 2, or single chain antibodies. In some embodiments, the anti-VEGFR1 antibody of the disclosure is a whole antibody or a full-length antibody.

In some embodiments, the anti-VEGFR1 antibody of the disclosure is an antibody fragment or an antigen binding domain that specifically binds to VEGFR1.

The terms “Full length antibodies”, “whole antibodies” and “intact antibodies” are used herein interchangeably to refer to an antibody having a structure similar to a native antibody. “Intact antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

As used herein, the term “Antibody fragment”, and “antigen binding fragment” refers to a molecule other than an intact antibody. Antigen binding fragments may be synthetic, enzymatically obtainable or genetically engineered polypeptides and include portions of an immunoglobulin that bind an antigen, such as a VH, a VL, a VH and aVL, a Fab, a Fab′, a F(ab′) 2, a Fd and a Fv fragments, a disulfide stabilized Fv fragment (dsFv), a (dsFv) 2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a domain antibody (dAb) consisting of one VH domain or one VL domain, a shark variable IgNAR domain, a camelized VH domain, a VHH domain, a minimal recognition unit consisting of the amino acid residues that mimic the CDRs of an antibody, such as a FR3-CDR3-FR4 portion, the HCDR1, the HCDR2 and/or the HCDR3 and the LCDR1, the LCDR2 and/or the LCDR3, an alternative scaffold that bind an antigen, a bivalent domain antibody, a multispecific protein comprising the antigen binding fragment or any other antibody fragment that binds to an antigen but does not comprise a complete antibody structure.

“dAb” or “dAb fragment” refers to an antibody fragment composed of a VH domain (Ward et al., Nature 341:544 546 (1989)).

“Fab” or “Fab fragment” refers to an antibody fragment composed of VH, CH1, VL and CL domains.

“F(ab′)2” or “F(ab′)2 fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody. Fv fragments lack the constant regions of Fab (CH1 and CL) regions. The VH and VL in Fv fragments are held together by non-covalent interactions.

Antigen binding fragments (such as VH and VL) may be linked together via a synthetic linker to form various types of single antibody designs where the VH/VL domains may be paired intramolecularly, or intermolecularly to form a monovalent antigen binding domain, such as single chain Fv (scFv) or diabody. In recombinant expression systems, the linker is a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to VEGFR1. The linker may be about 5-50 amino acids long.

“Single chain Fv” or “scFv” are fusion proteins comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. A scFv may have the VL and VH variable regions in either order, e.g., with respect to the N-terminal and C-terminal ends of the polypeptide, the scFv may comprise VL-linker-VH or may comprise VH-linker-VL.

Divalent or bivalent single chain variable fragments (di-scFv, bi-scFvs) can be engineered by linking two scFvs “. (scFv) 2” or “tandem scFv” or “bis-scFv” fragments refers to a fusion protein comprising two light chain variable regions (VL) and two heavy chain variable regions (VH), wherein the two VL and the two VH regions are contiguously linked via polypeptide linkers, and capable of being expressed as a single chain polypeptide. The two VL and two VH regions fused by peptide linkers form a bivalent molecule VLA-linker-VHA-linker-VLB-linker-VHB to form two binding sites, capable of binding two different antigens or epitopes concurrently. (ScFv) 2 can be expressed as a single chain polypeptide.

Any of the VH and the VL domains identified herein that bind VEGFR1 may be engineered into scFv format in either VH-linker-VL or VL-linker-VH orientation. Any of the VH and the VL domains identified herein may also be used to generate sc (Fv) 2 structures, such as VH-linker-VL-linker-VL-linker-VH, VH-linker-VL-linker-VH-linker-VL, VH-linker-VH-linker-VL-linker-VL, VL-linker-VH-linker-VH-linker-VL, VL-linker-VH-linker-VL-linker-VH or VL-linker-VL-linker-VH-linker-VH.

“Diabodies” are bivalent dimers formed from two chains, each containing a VH and a VL domain. The two domains within a chain are separated by a linker that is too short to facilitate intrachain dimerization leading to two chains dimerising in a head-to-tail arrangement. The linker may be a pentameric glycine-rich linker (G4S).

“VHH” refers to a single-domain antibody or nanobody, exclusively composed of the antigen binding domain of a heavy chain. A VHH single domain antibody lacks the light chain and the CH1 domain of the heavy chain of conventional Fab region. In some embodiments, the anti-VEGFR1 antibodies of the disclosure include Fv fragments, single chain scFv fragments (scFv), (scFv)₂, Fab, F(ab)₂, diabodies, VHH, dAb, Fd, Fv, or other single chain antibodies.

The anti-VEGFR1 antibody of the disclosure include chimeric, humanized or fully human antibodies that specifically bind to VEGFR1.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both.

Typically, a “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

Transgenic animals, such as mice, rat or chicken carrying human immunoglobulin (Ig) loci in their genome may be used to generate antigen binding fragments that bind VEGFR1, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (World Wide Web: regeneron.com), Harbour Antibodies (World Wide Web: harbourantibodies.com), Open Monoclonal Technology, Inc. (OMT) (World Wide Web: omtinc.net), KyMab (World Wide Web: kymab.com), Trianni (World Wide Web: trianni.com) and Ablexis (World Wide Web: ablexis.com) may be engaged to provide human antibodies directed against a selected antigen.

The antibody or antigen binding fragment thereof that bind VEGRFI generated by immunizing non-human animals may be humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding domains may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antigen binding domain.

In some embodiments, the disclosure provides an isolated antibody or antigen binding fragment thereof that binds VEGFR1 comprising the HCDR1, the HCDR2, the HCDR3, the LCDR1, the LCDR2 and the LCDR3 of
a. SEQ ID NOs: 7, 175, 9, 10, 11 and 12, respectively;
b. SEQ ID NOs: 7, 8, 9, 10, 11 and 12, respectively;
c. SEQ ID NOs: 13, 14, 15, 16, 17 and 18, respectively;
d. SEQ ID NOs: 19, 20, 21, 22, 23 and 24, respectively;
e. SEQ ID NOs: 25, 26, 27, 28, 29, and 30, respectively;
f. SEQ ID NOs: 71, 176, 73, 74, 75 and 76, respectively;
g. SEQ ID NOs: 71, 72, 73, 74, 75 and 76, respectively;
h. SEQ ID NOs: 77, 78, 79, 90, 81, and 82, respectively;
i. SEQ ID NOs: 83, 84, 85, 86, 87, and 88, respectively;
j. SEQ ID NOs: 89, 90, 91, 92, 93 and 94, respectively;
k. SEQ ID NOs: 95, 177, 97, 98, 99 and 100, respectively;
1. SEQ ID NOs: 95, 96, 97, 98, 99 and 100, respectively;
m. SEQ ID NOs: 101, 102, 103, 104, 105 and 106, respectively;
n. SEQ ID NOs: 107, 108, 109, 110, 111, and 112, respectively;
o. SEQ ID NOs: 113, 114, 115, 116, 117, and 118, respectively;
p. SEQ ID NOs: 119, 178, 121, 122, amino acid sequence LNS, and SEQ ID NO: 124, respectively;
q. SEQ ID NOs: 119, 120, 121, 122, amino acid sequence LNS, and SEQ ID NO: 124, respectively;
r. SEQ ID NOs: 125, 126, 127, 128, amino acid sequence FNF, and SEQ ID NO: 130, respectively;
s. SEQ ID NOs: 131, 132, 133, 134, amino acid sequence YD, and SEQ ID NO: 136, respectively; or
t. SEQ ID NOs: 137, 138, 139, 140, amino acid sequence FNS, and SEQ ID NO: 142, respectively.

In some embodiments, the antibody or antigen binding fragment thereof that binds VEGFR1 comprises the HCDR1, the HCDR2, the HCDR3, the LCDR1, the LCDR2, and the LCDR3 of SEQ ID NO: 7, 175, 9, 10, 11, and 12, respectively.

In some embodiments, the antibody or antigen binding fragment thereof that binds VEGFR1 comprises the HCDR1, the HCDR2, the HCDR3, the LCDR1, the LCDR2 and the LCDR3 of SEQ ID NO: 7, 8, 9, 10, 11 and 12, respectively.

In some embodiments, the antibody or antigen binding fragment thereof that binds VEGFR1 comprises the HCDR1, the HCDR2, the HCDR3, the LCDR1, the LCDR2 and the LCDR3 of SEQ ID NO: 13, 14, 15, 16, 17 and 18, respectively.

In some embodiments, the antibody or antigen binding fragment thereof that binds VEGFR1 comprises the HCDR1, the HCDR2, the HCDR3, the LCDR1, the LCDR2 and the LCDR3 of SEQ ID NO: 19, 20, 21, 22, 23 and 24, respectively.

In some embodiments, the antibody or antigen binding fragment thereof that binds VEGFR1 comprises the HCDR1, the HCDR2, the HCDR3, the LCDR1, the LCDR2 and the LCDR3 of SEQ ID NO: 25, 26, 27, 28, 29, and 30, respectively.

In some embodiments, the disclosure provides an isolated antibody or antigen binding fragment thereof that binds VEGFR1 comprising the HCDR1, the HCDR1, the HCDR3, the LCDR1, the LCDR2 and the LCDR3 of SEQ ID NOs: 25, 26, 27, 28, 29 and 30, respectively.

The disclosure also provides an isolated antibody or antigen binding fragment thereof comprising
a heavy chain complementarity determining region (HCDR) 1, a HCDR2 and a HCDR3 of a heavy chain variable region (VH) of SEQ ID NO: 31 and a light chain complementarity determining region (LCDR) 1, a LCDR2 and a LCDR3 of a light chain variable region (VL) of SEQ ID NO: 32; or the HCDR1, the HCDR2 and the HCDR3 of the VH of SEQ ID NO: 33 and the LCDR1, the LCDR2 and the LCDR3 of the VL of SEQ ID NO: 32; or the HCDR1, the HCDR2 and the HCDR3 of the VH of SEQ ID NO: 34 and the LCDR1, the LCDR2 and the LCDR3 of the VL of SEQ ID NO: 35; or the HCDR1, the HCDR2 and the HCDR3 of the VH of SEQ ID NO: 36 and the LCDR1, the LCDR2 and the LCDR3 of the VL of SEQ ID NO: 37; or the HCDR1, the HCDR2 and the HCDR3 of the VH of SEQ ID NO: 38 and the LCDR1, the LCDR2 and the LCDR3 of the VL of SEQ ID NO: 37; or the HCDR1, the HCDR2 and the HCDR3 of the VH of SEQ ID NO: 39 and the LCDR1, the LCDR2 and the LCDR3 of the VL of SEQ ID NO: 40. In some embodiments, the isolated antibody or antigen binding fragment thereof comprises the HCDR1, the HCDR2 and the HCDR3 of the VH of SEQ ID NO: 39 and the LCDR1, the LCDR2 and the LCDR3 of the VL of SEQ ID NO: 40 and wherein the antibody or antigen binding fragment thereof binds VEGFR1.

In some embodiments, the disclosure provides an isolated antibody or antigen binding fragment thereof comprising
a. the VH of SEQ ID NO: 31 and the VL of SEQ ID NO: 32;
b. the VH of SEQ ID NO: 33 and the VL of SEQ ID NO: 32;
c. the VH of SEQ ID NO: 34 and the VL of SEQ ID NO: 35;
d. the VH of SEQ ID NO: 36 and the VL of SEQ ID NO: 37;
e. the VH of SEQ ID NO: 38 and the VL of SEQ ID NO: 37;
f. the VH of SEQ ID NO: 39 and the VL of SEQ ID NO: 40; or
   and wherein the antibody or antigen binding fragment thereof binds VEGFR1.

In some embodiments, the antibody or antigen binding fragment thereof that binds VEGFR1 comprises the VH of SEQ ID NO: 31 and the VL of SEQ ID NO: 32.

In some embodiments, the antibody or antigen binding fragment thereof that binds VEGFR1 comprises the VH of SEQ ID NO: 33 and the VL of SEQ ID NO: 32.

In some embodiments, the antibody or antigen binding fragment thereof that binds VEGFR1 comprises the VH of SEQ ID NO: 34 and the VL of SEQ ID NO: 35.

In some embodiments, the antibody or antigen binding fragment thereof that binds VEGFR1 comprises the VH of SEQ ID NO: 36 and the VL of SEQ ID NO: 37.

In some embodiments, the antibody or antigen binding fragment thereof that binds VEGFR1 comprises the VH of SEQ ID NO: 38 and the VL of SEQ ID NO: 37.

In some embodiments, the antibody or antigen binding fragment thereof that binds VEGFR1 comprises the VH of SEQ ID NO: 39 and the VL of SEQ ID NO: 40.

In some embodiment, the disclosure also provides an isolated antibody or antigen binding fragment thereof comprising an amino acid sequence selected from the group consisting of SEQ ID NOs: 51, 52, 53, 54, 55, 56, 57, 58, 59 and 60.

In some embodiments, the disclosure provides an isolated antibody or antigen binding fragment thereof comprising an amino acid sequence of SEQ ID NO: 51.

In some embodiments, the disclosure also provides an isolated antibody or antigen binding fragment thereof comprising an amino acid sequence of SEQ ID NO: 52.

In some embodiments, the disclosure provides an isolated antibody or antigen binding fragment thereof comprising an amino acid sequence of SEQ ID NO: 53.

In some embodiments, the disclosure also provides an isolated antibody or antigen binding fragment thereof comprising an amino acid sequence of SEQ ID NO: 54.

In some embodiments, the disclosure provides an isolated antibody or antigen binding fragment thereof comprising an amino acid sequence of SEQ ID NO: 55.

In some embodiments, the disclosure also provides an isolated antibody or antigen binding fragment thereof comprising an amino acid sequence of SEQ ID NO: 56.

In some embodiments, the disclosure provides an isolated antibody or antigen binding fragment thereof comprising an amino acid sequence of SEQ ID NO: 57.

In some embodiments, the disclosure also provides an isolated antibody or antigen binding fragment thereof comprising an amino acid sequence of SEQ ID NO: 58.

In some embodiments, the disclosure provides an isolated antibody or antigen binding fragment thereof comprising an amino acid sequence of SEQ ID NO: 59.

In some embodiments, the disclosure also provides an isolated antibody or antigen binding fragment thereof comprising an amino acid sequence of SEQ ID NO: 60.

In some embodiments, the disclosure provides an isolated antibody or antigen binding fragment thereof comprising
a. a HC comprising the amino acid sequence of SEQ ID NO: 51 and a LC comprising the amino acid sequence of SEQ ID NO: 52;
b. a HC comprising the amino acid sequence of SEQ ID NO: 53 and a LC comprising the amino acid sequence of SEQ ID NO: 52;
c. a HC comprising the amino acid sequence of SEQ ID NO: 54 and a LC comprising the amino acid sequence of SEQ ID NO: 55;
d. a HC comprising the amino acid sequence of SEQ ID NO: 56 and a LC comprising the amino acid sequence of SEQ ID NO: 57;
e. a HC comprising the amino acid sequence of SEQ ID NO: 58 and a LC comprising the amino acid sequence of SEQ ID NO: 57; or
f. a HC comprising the amino acid sequence of SEQ ID NO: 59 and a LC comprising the amino acid sequence of SEQ ID NO: 60.

In some embodiments, the disclosure provides an isolated antibody or antigen binding fragment thereof comprising
a HCDR1, a HCDR2, a HCDR3, a LCDR1, a LCDR2 and a LCDR3 of SEQ ID NOs: 7, 175, 9, 10, 11, and 12, respectively;
a VH of SEQ ID NO: 31 and a VL of SEQ ID NO: 32; and/or
a HC of SEQ ID NO: 51 and a LC of SEQ ID NO: 52;
and wherein the antibody or antigen binding fragment thereof binds VEGFR1.

In some embodiments, the disclosure provides an isolated antibody or antigen binding fragment thereof comprising
a HCDR1, a HCDR2, a HCDR3, a LCDR1, a LCDR2 and a LCDR3 of SEQ ID NOs: 7, 8, 9, 10, 11, and 12, respectively;
a VH of SEQ ID NO: 33 and a VL of SEQ ID NO: 32; and/or
a HC of SEQ ID NO: 53 and a LC of SEQ ID NO: 52;
and wherein the antibody or antigen binding fragment thereof binds VEGFR1.

In some embodiments, the disclosure provides an isolated antibody or antigen binding fragment thereof comprising
a HCDR1, a HCDR2, a HCDR3, a LCDR1, a LCDR2 and a LCDR3 of SEQ ID NOs: 13, 14, 15, 16, 17 and 18, respectively;
a VH of SEQ ID NO: 34 and a VL of SEQ ID NO: 35; and/or
a HC of SEQ ID NO: 54 and a LC of SEQ ID NO: 55;
and wherein the antibody or antigen binding fragment thereof binds VEGFR1.

In some embodiments, the disclosure provides an isolated antibody or antigen binding fragment thereof comprising
a HCDR1, a HCDR2, a HCDR3, a LCDR1, a LCDR2 and a LCDR3 of SEQ ID NOs: 19, 20, 21, 22, 23, and 24, respectively;
a VH of SEQ ID NO: 36 and a VL of SEQ ID NO: 37; and/or
a HC of SEQ ID NO: 56 and a LC of SEQ ID NO: 57;
and wherein the antibody or antigen binding fragment thereof binds VEGFR1.

In some embodiments, the disclosure provides an isolated antibody or antigen binding fragment thereof comprising
a HCDR1, a HCDR2, a HCDR3, a LCDR1, a LCDR2 and a LCDR3 of SEQ ID NOs: 19, 20, 21, 22, 23, and 24, respectively;
a VH of SEQ ID NO: 38 and a VL of SEQ ID NO: 37; and/or
a HC of SEQ ID NO: 58 and a LC of SEQ ID NO: 57;
and wherein the antibody or antigen binding fragment thereof binds VEGFR1.

In some embodiments, the disclosure provides an isolated antibody or antigen binding fragment thereof comprising
a HCDR1, a HCDR2, a HCDR3, a LCDR1, a LCDR2 and a LCDR3 of SEQ ID NOs: 25, 26, 27, 28, 29, and 30, respectively;
a VH of SEQ ID NO: 39 and a VL of SEQ ID NO: 40; and/or
a HC of SEQ ID NO: 59 and a LC of SEQ ID NO: 60;
and wherein the antibody or antigen binding fragment thereof binds VEGFR1.

In some embodiments, the disclosure provides an isolated antibody or antigen binding fragment thereof comprising
a VH of SEQ ID NO: 31, a VL of SEQ ID NO: 32,
a HC of SEQ ID NO: 51 and a LC of SEQ ID NO: 52;
and the antibody or antigen binding fragment thereof binds VEGFR1.

In some embodiments, the disclosure provides an isolated antibody or antigen binding fragment thereof comprising
a VH of SEQ ID NO: 33, a VL of SEQ ID NO: 32,
a HC of SEQ ID NO: 53 and a LC of SEQ ID NO: 52;
and the antibody or antigen binding fragment thereof binds VEGFR1.

In some embodiments, the disclosure provides an isolated antibody or antigen binding fragment thereof comprising
a VH of SEQ ID NO: 34, a VL of SEQ ID NO: 35,
a HC of SEQ ID NO: 54 and a LC of SEQ ID NO: 55;
and the antibody or antigen binding fragment thereof binds VEGFR1.

In some embodiments, the disclosure provides an isolated antibody or antigen binding fragment thereof comprising
a VH of SEQ ID NO: 36, a VL of SEQ ID NO: 37,
a HC of SEQ ID NO: 56 and a LC of SEQ ID NO: 57;
and the antibody or antigen binding fragment thereof binds VEGFR1.

In some embodiments, the disclosure provides an isolated antibody or antigen binding fragment thereof comprising
a VH of SEQ ID NO: 38, a VL of SEQ ID NO: 37,
a HC of SEQ ID NO: 58 and a LC of SEQ ID NO: 57;
and the antibody or antigen binding fragment thereof binds VEGFR1.

In some embodiments, the disclosure provides an isolated antibody or antigen binding fragment thereof comprising
a VH of SEQ ID NO: 39, a VL of SEQ ID NO: 40,
a HC of SEQ ID NO: 59 and a LC of SEQ ID NO: 60;
and the antibody or antigen binding fragment thereof binds VEGFR1.

In some embodiments, the isolated antibody or isolated antigen binding fragment thereof is a full-length antibody. In some embodiments, the isolated antibody or isolated antigen binding domain thereof is an antibody fragment or antigen binding fragment

Homologous Antibodies and Antigen Binding Fragment Thereof with Conservative Substitutions

Derivatives, homologous antigen binding domains, functional equivalents, or variants of said antibody or antigen binding fragment thereof are also object of the disclosure. The antibodies of the present disclosure also include homologous antibodies, homologous antigen binding domains, functional equivalents or variants of the disclosed antibody or antigen binding fragment thereof that bind VEGFR1, that include polypeptides with amino acid sequences substantially identical to the amino acid sequence of the variable domain or hypervariable domain of the antibodies of the present disclosure or polypeptides with conservative substitutions. The homologous antibodies and antigen binding domain, functional equivalents or variants of the disclosure have sufficient homology with the sequences of said antibody or antigen binding fragment thereof that binds VEGFR1 and are functionally similar to the unmodified anti-VEGFR1 antibody to retain binding to VEGFR1 or retain at least one of the activities of the unmodified antibody.

The term “antibody derivative”, “homologous antigen binding domain”, “functional equivalents” or “variants” refer to antibodies comprising one or more mutations, substitutions, deletions and/or additions of one or more amino acid residues. Such an addition, substitution or deletion can be located at any position in the molecule. In the case where several amino acids have been added, substituted, or deleted, any combination of addition, substitution or deletion can be considered, on condition that the resulting antibody still has at least the advantageous properties of the antibody of the invention.

Sequences of the disclosure may comprise amino acid sequences with at least 80% identity or homology to the sequences described above. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding domains that bind VEGFR1 of the disclosure. Variants of the antigen binding domains that bind VEGFR1 comprising 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acid substitutions in the antigen binding domain that bind VEGFR1 are within the scope of the disclosure, as long as they retain or have improved functional properties when compared to the parent antigen binding domains. Functional equivalents or variants of the antigen binding domains that bind VEGFR1 include one or more deletions and/or additions of one or more amino acid residues. Such an addition, substitution or deletion can be located at any position in the molecule. In the case where several amino acids have been added, substituted or deleted, any combination of addition, substitution or deletion can be considered, on condition that the resulting antibody still has at least the advantageous properties of the antibody of the invention.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 31 and a VL of at least 80% (e.g. at least 85%, at least 90%, at least 95%, or at least 99%) identical to the VL of SEQ ID NO: 32.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 33 and a VL of at least 80% (e.g. at least 85%, at least 90%, at least 95%, or at least 99%) identical to the VL of SEQ ID NO: 32.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 34 and a VL of at least 80% (e.g. at least 85%, at least 90%, at least 95%, or at least 99%) identical to the VL of SEQ ID NO: 35.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 36 and a VL of at least 80% (e.g. at least 85%, at least 90%, at least 95%, or at least 99%) identical to the VL of SEQ ID NO: 37.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 38 and a VL of at least 80% (e.g. at least 85%, at least 90%, at least 95%, or at least 99%) identical to the VL of SEQ ID NO: 37.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 39 and a VL of at least 80% (e.g. at least 85%, at least 90%, at least 95%, or at least 99%) identical to the VL of SEQ ID NO: 40.

The disclosure also provides an isolated antibody or antigen binding fragment thereof comprising a VH which is at least 80% (e.g. at least 85%, at least 90%, at least 95%, at least 99% or 100%) identical to the VH of SEQ ID NO: 39 and a VL which is at least 80% (e.g. at least 85%, at least 90%, at least 95%, at least 99% or 100%) identical to the VL of SEQ ID NO: 40.

In some embodiments, the antibody or antigen binding fragment thereof that binds VEGFR1 comprises a VH which is at least 80% (e.g. at least 85%, at least 90%, at least 95% or at least 99%) identical to the VH of SEQ ID NO: 39 and a VL of SEQ ID NO: 40.

In some embodiments, the antibody or antigen binding fragment thereof that binds VEGFR1 comprises a VH of SEQ ID NO: 39 and a VL which is at least 80% (e.g. at least 85%, at least 90%, at least 95% or at least 99%) identical to the VL of SEQ ID NO: 40.

In some embodiments, the antibody or antigen binding fragment thereof that binds VEGFR1 comprises a VH which is at least 95% identical to the VH of SEQ ID NO: 39 and a VL which is at least 95% identical to the VL of SEQ ID NO: 40.

In some embodiments, the antibody or antigen binding fragment thereof that binds

VEGFR1 comprises a VH which is at least 95% identical to the VH of SEQ ID NO: 39 and a VL which is at least 99% identical to the VL of SEQ ID NO: 40.

In some embodiments, the antibody or antigen binding fragment thereof that binds VEGFR1 comprises a VH which is at least 99% identical to the VH of SEQ ID NO: 39 and a VL which is at least 99% identical to the VL of SEQ ID NO: 40.

In some embodiments, the antibody or antigen binding fragment thereof that binds VEGFR1 comprises a VH which is at least 99% identical to the VH of SEQ ID NO: 39 and a VL which is at least 95% identical to the VL of SEQ ID NO: 40.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 39 and a VL of at least 80% (e.g. at least 85%, at least 90%, at least 95%, or at least 99%) identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

The disclosure also provides an isolated antibody or antigen binding fragment thereof comprising a VH which is at least 80% (e.g. at least 85%, at least 90%, at least 95%, at least 99% or 100%) identical to the VH of SEQ ID NO: 39 and a VL which is at least 80% (e.g. at least 85%, at least 90%, at least 95%, at least 99% or 100%) identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody or antigen binding fragment thereof that binds VEGFR1 comprises a VH which is at least 80% (e.g. at least 85%, at least 90%, at least 95% or at least 99%) identical to the VH of SEQ ID NO: 39 and a VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody or antigen binding fragment thereof that binds VEGFR1 comprises a VH of SEQ ID NO: 39 and a VL which is at least 80% (e.g. at least 85%, at least 90%, at least 95% or at least 99%) identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody or antigen binding fragment thereof that binds VEGFR1 comprises a VH which is at least 95% identical to the VH of SEQ ID NO: 39 and a VL which is at least 95% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody or antigen binding fragment thereof that binds VEGFR1 comprises a VH which is at least 95% identical to the VH of SEQ ID NO: 39 and a VL which is at least 99% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody or antigen binding fragment thereof that binds VEGFR1 comprises a VH which is at least 99% identical to the VH of SEQ ID NO: 39 and a VL which is at least 99% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody or antigen binding fragment thereof that binds VEGFR1 comprises a VH which is at least 99% identical to the VH of SEQ ID NO: 39 and a VL which is at least 95% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiment, the disclosure also provides an isolated antibody or antigen binding fragment thereof comprising an amino acid sequence at least 80% (e.g. at least 85%, at least 90%, at least 95%, at least 99% or 100%) identical to the amino acid sequence of SEQ ID NO: 59 and wherein the antibody or antigen binding fragment thereof binds VEGFR1.

In some embodiments, the disclosure also provides an isolated antibody or antigen binding fragment thereof comprising an amino acid sequence at least 80% (e.g. at least 85%, at least 90%, at least 95%, at least 99% or 100%) identical to the amino acid sequence of SEQ ID NO: 60; and wherein the antibody or antigen binding fragment thereof binds VEGFR1.

In some embodiments, an isolated antibody or antigen binding fragment thereof of the application comprises a heavy chain (HC) having an amino acid sequence at least 80% (e.g. at least 85%, at least 90%, at least 95%, or at least 99%) identical to the amino acid sequence of SEQ ID NO: 51, SEQ ID NO: 53, SEQ ID NO: 54, SEQ ID NO: 56, SEQ ID NO: 58 or SEQ ID NO: 59, and a light chain (LC) having an amino acid sequence at least 80% (e.g. at least 85%, at least 90%, at least 95%, or at least 99%) identical to the amino acid sequence of SEQ ID NO: 52, SEQ ID NO: 55, SEQ ID NO: 57 or SEQ ID NO: 60.

The disclosure also provides an isolated antibody or antigen binding fragment thereof comprising a HC which is at least 80% (e.g. at least 85%, at least 90%, at least 95%, at least 99% or 100%) identical to the HC of SEQ ID NO: 59 and a LC which is at least 80% (e.g. at least 85%, at least 90%, at least 95%, at least 99% or 100%) identical to the LC of SEQ ID NO: 60.

In some embodiments, the antibody or antigen binding fragment thereof that binds VEGFR1 comprises a HC which is at least 80% (e.g. at least 85%, at least 90%, at least 95% or at least 99%) identical to the HC of SEQ ID NO: 59 and a LC of SEQ ID NO: 60.

In some embodiments, the antibody or antigen binding fragment thereof that binds VEGFR1 comprises a HC of SEQ ID NO: 59 and a LC which is at least 80% (e.g. at least 85%, at least 90%, at least 95% or at least 99%) identical to the LC of SEQ ID NO: 60.

In some embodiments, the antibody or antigen binding fragment thereof that binds VEGFR1 comprises a HC which is at least 95% identical to the HC of SEQ ID NO: 59 and a LC which is at least 95% identical to the LC of SEQ ID NO: 60.

In some embodiments, the antibody or antigen binding fragment thereof that binds VEGFR1 comprises a HC which is at least 95% identical to the HC of SEQ ID NO: 59 and a LC which is at least 99% identical to the LC of SEQ ID NO: 60.

In some embodiments, the antibody or antigen binding fragment thereof that binds VEGFR1 comprises a HC which is at least 99% identical to the HC of SEQ ID NO: 59 and a LC which is at least 99% identical to the LC of SEQ ID NO: 60.

In some embodiments, the antibody or antigen binding fragment thereof that binds VEGFR1 comprises a HC which is at least 99% identical to the HC of SEQ ID NO: 59 and a LC which is at least 95% identical to the LC of SEQ ID NO: 60.

The disclosure also provides an isolated antibody or antigen binding fragment thereof comprising a HC which is at least 80% (e.g. at least 85%, at least 90%, at least 95%, at least 99% or 100%) identical to the HC of SEQ ID NO: 59 and a LC which is at least 80% (e.g. at least 85%, at least 90%, at least 95%, at least 99% or 100%) identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody that binds VEGFR1 comprises a HC which is at least 80% (e.g. at least 85%, at least 90%, at least 95% or at least 99%) identical to the HC of SEQ ID NO: 59 and a LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody that binds VEGFR1 comprises a HC of SEQ ID NO: 59 and a LC which is at least 80% (e.g. at least 85%, at least 90%, at least 95% or at least 99%) identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody that binds VEGFR1 comprises a HC which is at least 95% identical to the HC of SEQ ID NO: 59 and a LC which is at least 95% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody that binds VEGFR1 comprises a HC which is at least 95% identical to the HC of SEQ ID NO: 59 and a LC which is at least 99% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody that binds VEGFR1 comprises a HC which is at least 99% identical to the HC of SEQ ID NO: 59 and a LC which is at least 99% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

In some embodiments, the antibody that binds VEGFR1 comprises a HC which is at least 99% identical to the HC of SEQ ID NO: 59 and a LC which is at least 95% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively.

The disclosure also provides an isolated antibody comprising a HC which is at least 80% (e.g. at least 85%, at least 90%, at least 95%, at least 99% or 100%) identical to the HC of SEQ ID NO: 59 and a LC which is at least 80% (e.g. at least 85%, at least 90%, at least 95%, at least 99% or 100%) identical to the LC of SEQ ID NO: 60, wherein the antibody or antigen binding fragment comprises a VH of SEQ ID NO: 39 and a VL of SEQ ID NO: 40.

In some embodiments, the antibody that binds VEGFR1 comprises a HC which is at least 80% (e.g. at least 85%, at least 90%, at least 95% or at least 99%) identical to the HC of SEQ ID NO: 59 and a LC of SEQ ID NO: 60, wherein the antibody or antigen binding fragment comprises a VH of SEQ ID NO: 39 and a VL of SEQ ID NO: 40.

In some embodiments, the antibody that binds VEGFR1 comprises a HC of SEQ ID NO: 59 and a LC which is at least 80% (e.g. at least 85%, at least 90%, at least 95% or at least 99%) identical to the LC of SEQ ID NO: 60, wherein the antibody or antigen binding fragment comprises a VH of SEQ ID NO: 39 and a VL of SEQ ID NO: 40.

In some embodiments, the antibody that binds VEGFR1 comprises a HC which is at least 95% identical to the HC of SEQ ID NO: 59 and a LC which is at least 95% identical to the LC of SEQ ID NO: 60, wherein the antibody or antigen binding fragment comprises a VH of SEQ ID NO: 39 and a VL of SEQ ID NO: 40.

In some embodiments, the antibody that binds VEGFR1 comprises a HC which is at least 95% identical to the HC of SEQ ID NO: 59 and a LC which is at least 99% identical to the LC of SEQ ID NO: 60, wherein the antibody or antigen binding fragment comprises a VH of SEQ ID NO: 39 and a VL of SEQ ID NO: 40.

In some embodiments, the antibody that binds VEGFR1 comprises a HC which is at least 99% identical to the HC of SEQ ID NO: 59 and a LC which is at least 99% identical to the LC of SEQ ID NO: 60, wherein the antibody or antigen binding fragment comprises a VH of SEQ ID NO: 39 and a VL of SEQ ID NO: 40.

In some embodiments, the antibody that binds VEGFR1 comprises a HC which is at least 99% identical to the HC of SEQ ID NO: 59 and a LC which is at least 95% identical to the LC of SEQ ID NO: 60, wherein the antibody or antigen binding fragment comprises a VH of SEQ ID NO: 39 and a VL of SEQ ID NO: 40.

The terms “identical” or percent “identity,” in the context of two or more nucleic acids or polypeptide sequences (e.g., anti-VEGFR1 antibodies and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection. The percent (%) amino acid sequence identity with respect to a reference polypeptide is defined as the percentage of amino acid residues in a given sequence that are identical to the amino acid residues in the reference polypeptide sequence. The percent (%) identity between the two sequences is a function of the number of identical positions shared by the sequences (i.e., % identity=number of identical positions/total number of positions ×100), taking into account the number of gaps, and the length of each gap, which need to be introduced for optimal alignment of the two sequences. The percent identity between two amino acid sequences may be determined using various the algorithms that are within the skill in the art, using publicly available software such as BLAS, BLAST-2, ALIGN. Megalin (DNASTAR) or the GAP program available in the GCG software package.

A polypeptide is typically substantially identical to a second polypeptide, for example, where the two peptides differ only by conservative substitutions. The antibodies of the present disclosure also include those for which binding characteristics, functional or physical properties have been improved by direct mutations. In some embodiments, variant antigen binding domains that bind VEGFR1 comprise one or two conservative substitutions in any of the CDR regions, while retaining desired functional properties of the parent antigen binding fragments that bind VEGFR1.

“Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

Species cross reactivity

In some embodiments, the anti-VEGFR1 antibody or antigen binding fragment thereof binds to human, mouse, rat and cynomolgus monkey VEGFR1. In some embodiments, the anti-VEGFR1 antibody or antigen binding fragment thereof of the disclosure is a species cross-reactive antibody able to bind human, mouse, rat and cynomolgus monkey VEGFR1 with similar affinities providing significant value to the anti-VEGFR1 antibody disclosed herein.

In some embodiments, the anti-VEGFR1 antibody or antigen binding fragment thereof binds to human and mouse VEGFR1. In some embodiments, the anti-VEGFR1 antibody or antigen binding fragment thereof binds to human and rat VEGFR1. In some embodiments, the anti-VEGFR1 antibody or antigen binding fragment thereof binds to human, mouse and rat VEGFR1. In some embodiments, the anti-VEGFR1 antibody or antigen binding fragment thereof binds to human, mouse, and cynomolgus monkey VEGFR1. In some embodiments, the anti-VEGFR1 antibody or antigen binding fragment thereof binds to human, rat and cynomolgus monkey VEGFR1. In some embodiments, the anti-VEGFR1 antibody or antigen binding fragment thereof binds to human VEGFR1.

One way to quantify the extent of species cross-reactivity of an antibody is as the fold difference in its affinity for an antigen of one species compared with an antigen of another species, e.g., fold difference in affinity for human VEGFR1 versus mouse VEGFR1, versus rat VEGFR1 or versus cynomolgus monkey VEGFR1. Affinity may be quantified as K_{D}, referring to the equilibrium dissociation constant of the antibody-antigen reaction as determined by SPR as described elsewhere herein. A species cross-reactive anti-VEGFR1 antibody may have a fold-difference in affinity for binding human and mouse VEGFR1, human and rat VEGFR1 or human and cynomolgus monkey VEGFR1 that is 500-fold or less, 250-fold or less, 100-fold or less, 50-fold or less, 25-fold or less, 10-fold or less, or 5-fold or less. To put it another way, the K_{D} of binding human VEGFR1 may be within 500-fold, 250-fold, 100-fold, 50-fold, 25-fold, 10-fold, or 5-fold of the K_{D} of binding to mouse, rat or cynomolgus monkey VEGFR1. In some embodiments, an antibody that binds human VEGFR1 may binds to mouse, rat or cynomolgus monkey with an affinity no weaker than 500-fold weaker than the affinity to human, e.g. with an affinity no weaker than 500-fold, 250-fold, 100-fold, 50-fold, 25-fold, 10-fold or 5-fold than the affinity to human VEGFR1. In some embodiments, an antibody that binds human VEGFR1 may binds to mouse, rat or cynomolgus monkey VEGFR1 with an affinity no stronger than 500-fold stronger than the affinity to human, e.g. with an affinity no stronger than 500-fold, 250-fold, 100-fold, 50-fold, 25-fold, 10-fold or 5-fold than the affinity to human VEGFR1. Antibodies can also be considered cross-reactive if the K_{D} for binding antigen of two different species meets a threshold value, e.g., if the K_{D} of binding human VEGFR1 and the K_{D} of binding mouse, rat or cynomolgus monkey VEGFR1 are 10 nM or less, 5 nM or less, 1 nM or less. The K_{D} may be 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less, 0.01 nM or less, 0.001 nM or less.

An alternative measure of cross-reactivity for binding human VEGFR1 and mouse, rat or cynomolgus monkey VEGFR1 is the ability of the antibody to block ligand binding to VEGR1, such as VEGFA binding or PlGF binding. A species cross reactive anti-VEGFR1 antibody may have an IC50 for blocking VEGFA binding or PlGF binding to human VEGFR1 that is within 25-fold, 20-fold, 15-fold, 10-fold or 5-fold of the IC50 for blocking binding of VEGFA or binding of PlGF to mouse, rat or cynomolgus monkey. As another alternative, a cross-species reactive antibody may have the ability to modulate circulating or tissue VEGFA or circulating or tissue PlGF levels in an animal, e.g. an antibody that binds human VEGFR1 may modulate circulating or tissue VEGFA or PlGF levels in mouse, rat and/or cynomolgus monkey.

Cross-species reactive antibodies enable for translational studies across several species and can help demonstrate efficacy and pharmacodynamics properties in established preclinical disease model without the need of surrogate antibodies in the development phase. Earlier anti-VEGFR1 antibodies reported were either human or mouse specific and showed poor cross-species reactivity.

Generating monoclonal antibodies that are species cross reactive and bind epitopes that are conserved between human and other species is challenging. The cross-species reactive anti-VEGFR1 antibody of the disclosure was generated through a unique immunization approach comprising (1) selection of both human and mouse VEGFR1 antigens comprising the VEGFR1 D2 and D3 domain, (2) selection of chicken, a phylogenetically distant immunization species, (3) immunization boosts alternating between human VEGFR1 D2-D3 and mouse VEGFR1 D2-D3 and (4) careful monitoring and selection of serum titers showing reactivity against both human and mouse VEGFR1.

In some embodiments, the anti-VEGFR1 antibody of the disclosure is able to bind to an epitope within the sequence of amino acid residues 130 to 331 of human VEGFR1 (Uniprot Accession #P17948). In some embodiments, the anti-VEGFR1 antibody of the disclosure recognizes and binds to an epitope within SEQ ID NO: 173. In some embodiments, the anti-VEGFR1 antibody of the disclosure recognizes and binds to an epitope within SEQ ID NO: 4.

In some embodiments, the anti-VEGFR1 antibody binds to an epitope on VEGFR1 set forth in SEQ ID NO: 143 (FPLDTL) and SEQ ID NO: 144 (EIGL). In some embodiments, the anti-VEGFR1 antibody binds to an epitope on VEGFR1 set forth in SEQ ID NO: 144.

In some embodiments, an isolated antibody or antigen binding fragment thereof binds specifically to a human VEGFR1 and is also capable of binding specifically to a VEGFR1 from at least one non-human species selected from the group consisting of cynomolgus monkey, mouse, and rat. In certain embodiments, an isolated antibody or antigen binding fragment thereof of the application binds specifically to a human VEGFR1, mouse VEGFR1, and cynomolgus monkey VEGFR1, with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

“Epitope” refers to a portion of an antigen to which an antibody specifically binds. Epitopes typically consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Paratope” refers to a portion of an antibody to which an antigen specifically binds. A paratope can be linear in nature or can be discontinuous, formed by a spatial relationship between non-contiguous amino acids of an antibody rather than a linear series of amino acids. A “light chain paratope” and a “heavy chain paratope” or “light chain paratope amino acid residues” and “heavy chain paratope amino acid residues” refer to antibody light chain and heavy chain residues in contact with an antigen, respectively, or in general, “antibody paratope residues” refer to those antibody amino acids that are in contact with antigen.

The determination of the residues within hVEGFR1 to which an antibody binds may be determined by any techniques known to a person of ordinary skill in the art. In some embodiments, the residues within hVEGFR1 to which an antibody binds are determined by H/D exchange assay. In an H/D exchange assay, recombinantly expressed hVEGFR1 is incubated in the presence or absence of the antibody in deuterated water for predetermined times resulting in deuterium incorporation at exchangeable hydrogen atoms which are unprotected by the antibody, followed by protease digestion of the protein and analyses of the peptide fragments using LC-MS. H/D exchange assay can be performed using known protocols. In some embodiments, the H/D exchange mixture is quenched by the addition of a quenching buffer (e.g. 8 M urea, 1M TCEP, pH 3.0) before being passed over an equilibrated immobilized pepsin/FPXIII column at room temperature (e.g. 600 μL/min). The peptic fragments are then loaded onto a reverse phase trap column (e.g. at 600 μL/min) and desalted (e.g. for 1 min at 600 μL), separated (e.g. on a C18 column) and analyzed by mass spectrometry (e.g. using an LTQ™ Orbitrap Fusion Lumos mass spectrometer (Thermo Fisher Scientific) with the capillary temperature at 275° C., resolution 150,000, and mass range (m/z) 300-1,800).

Antibodies blocking VEGFA binding to VEGFR1

In some embodiments, the anti-VEGFR1 antibody of the disclosure is able to recognize and bind to an epitope in the D2-D3 domain of VEGFR1 and prevent binding of vascular endothelial growth factor A (VEGFA).

In the kidney, VEGFA made by glomerular podocytes and tubular epithelial cells is essential to maintain glomerular integrity, renal microvasculature, and function. The crucial role of VEGFA in kidney development, structure and function is manifested in the severe phenotypes observed in conditional or inducible VEGFA knockout (KO) mice. In chronic kidney disease (CKD) patients, mRNA microarray analyses of patient kidney biopsies showed decreased VEGFA expression and the level of VGFA expression in glomeruli and tubulointerstitium is correlated with eGFR, proteinuria or vascular rarefaction (Bortoloso, Del Prete et al. 2004, Martini, Nair et al. 2014, Pan, Jiang et al. 2018). VEGFA functions through two endothelial restricted receptors: 1) the major signaling receptor VEGFR2, and 2) the decoy receptor VEGFR1, which sequesters VEGFA from VEGFR2 with 10-fold higher binding affinity but has weak or undetectable tyrosine kinase activity (TK). The anti-VEGFR1 of the disclosure are aimed at restoring impaired VEGFA level and VEGFA activity in the kidney, by blocking the sequestration of VEGFA by its decoy receptor VEGFR1. The anti-VEGFR1 antibody of the disclosure will block VEGFA sequestration and increase local VEGFA availability, which in turn will enable endothelial protection, preservation of glomerular filtration barrier (GFB) and renal function restoration.

### Conjugation and Fc Engineering

In addition to the modification set forth above, the anti-VEGFR1 antibody or antigen binding fragment thereof of the present disclosure and their functional equivalents may be conjugated to other antibodies, proteins, antigen binding fragments or alternative scaffolds.

The anti-VEGFR1 antibodies, antigen binding fragment thereof, and the functional equivalents of the disclosure may be conjugated to a half-life extending moiety. Exemplary half-life extending moieties are albumin, albumin variants, albumin-binding proteins and/or domains, transferrin and fragments and analogues thereof, immunoglobulins (Ig) or fragments thereof, such as Fc regions. Amino acid sequences of the aforementioned half-life extending moieties are known. Additional half-life extending moieties that may be conjugated to the anti-VEGFR1 antibodies, antigen binding fragment thereof, and the functional equivalents of the disclosure include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the antibody or antigen binding fragment of the disclosure and may be generated by standard cloning and expression techniques. Alternatively, well known chemical coupling methods may be used to attach the moieties to the recombinantly produced antibody or antigen binding fragment of the disclosure.

A pegyl moiety may for example be conjugated to the antibody or antigen binding fragment thereof that bind VEGFR1 by incorporating a cysteine residue to the C-terminus of the antibody or antigen binding fragment that bind VEGFR1 or engineering cysteines into residue positions that face away from the VEGFR1 binding site and attaching a pegyl group to the cysteine using well known methods.

In some embodiments, the half-life extending moiety is albumin.

In some embodiments, the half-life extending moiety is the albumin binding domain.

In some embodiments, the half-life extending moiety is transferrin.

In some embodiments, the half-life extending moiety is polyethylene glycol.

In some embodiments, conjugation further include, but are not limited to conjugation to detectable receptor molecules.

In some embodiments, the half-life extending moiety is an Ig constant region or a fragment of the Ig constant region.

In some embodiments, the half-life extending moiety is an Ig.

In some embodiments, the half-life extending moiety is a fragment of the Ig.

In some embodiments, the half-life extending moiety is the Ig constant region.

In some embodiments, the half-life extending moiety is the fragment of the Ig constant region.

In some embodiments, the half-life extending moiety is the Fc region.

The Ig constant region or the fragment of the Ig constant region, such as the Fc region present in the antibody or antigen binding fragment thereof of the disclosure may be of any allotype or isotype, i.e., IgG1, IgG2, IgG3, IgG4, IgM, IgA and IgE.

In some embodiments, the Ig constant region or the fragment of the Ig constant region is an IgG1 isotype.

In some embodiments, the Ig constant region or the fragment of the Ig constant region is an IgG2 isotype.

In some embodiments, the Ig constant region or the fragment of the Ig constant region is an IgG3 isotype.

In some embodiments, the Ig constant region or the fragment of the Ig constant region is an IgG4 isotype.

It is expected that allotype has no influence on properties of the Ig constant region, such as binding or Fc-mediated effector functions. Immunogenicity of therapeutic proteins comprising Ig constant regions of fragments thereof is associated with increased risk of infusion reactions and decreased duration of therapeutic response (Baert et al., (2003) N Engl J Med 348:602-08). The extent to which therapeutic proteins comprising Ig constant regions of fragments thereof induce an immune response in the host may be determined in part by the allotype of the Ig constant region (Stickler et al., (2011) Genes and Immunity 12:213-21). Ig constant region allotype is related to amino acid sequence variations at specific locations in the constant region sequences of the antibody.

The antibody or antigen binding fragment thereof of the present disclosure and their functional equivalents may be conjugated to an Ig constant region or to the fragment of an Ig constant region to modulate the antibody or antigen binding fragment effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), inhibitory FcγRIIb and/or to FcRn.

In some embodiments, the antibody or antigen binding fragment thereof that binds VEGFR1 is conjugated to an Ig constant region or the fragment of the Ig constant region comprising at least one mutation in the Ig constant region or in the fragment of the Ig constant region.

In some embodiments, the at least one mutation is in the Fc region.

In some embodiments, the antibody or antigen binding fragment thereof that binds VEGFR1 is conjugated to an Ig constant region or to the fragment of the Ig constant region comprises at least one, two, three, four, five, six, seven, eight, nine, ten, eleven, twelve, thirteen, fourteen or fifteen mutations in the Fc region.

The neonatal Fc receptor (FcRn) plays a central role in the cellular trafficking and serum half-life of IgGs. In some embodiments, the antibody or antigen binding fragment thereof that binds VEGFR1 is conjugated to an Ig constant region or to the fragment of the Ig constant region comprising at least one mutation in the Fc region that modulates binding of the antibody or antigen binding fragment to FcRn and modulates the half-life of the antibody or antigen binding fragment.

In some embodiments, the Ig constant region or the fragment of the first Ig constant region comprises at least one mutation that modulates a half-life of the isolated antibody or antigen binding fragment thereof.

Fc positions that may be mutated to modulate half-life (e.g. binding to FcRn) include positions 250, 252, 253, 254, 256, 257, 307, 376, 380, 428, 434 and 435. Exemplary mutations that may be made singularly or in combination are mutations T250Q, M252Y, I253A, S254T, T256E, P257I, T307A, D376V, E380A, M428L, H433K, N434S, N434A, N434H, N434F, H435A and H435R. Exemplary singular or combination mutations that may be made to increase the half-life are mutations M428L/N434S, M252Y/S254T/T256E, T250Q/M428L, N434A and T307A/E380A/N434A. In some embodiments, the at least one mutation that modulates the half-life of the antibody or antigen binding fragment thereof of the disclosure and their functional equivalents is selected from the group consisting of H435A, P257I/N434H, D376V/N434H, M252Y/S254T/T256E/H433K/N434F, T308P/N434A and H435R, wherein residue numbering is according to the EU index

In some embodiments, the antibody or antigen binding fragment thereof that binds VEGFR1 is conjugated to the Ig constant region or to the fragment of the Ig constant region comprising M252Y/S254T/T256E mutation.

In some embodiments, the antibody or antigen binding fragment of the disclosure and their function equivalents is conjugated to an Ig constant region or to the fragment of the Ig constant region comprising at least one mutation in the Fc region that reduces binding of the protein to an activating Fcγ receptor (FcγR) and/or reduces Fc effector functions such as Clq binding, complement dependent cytotoxicity (CDC), antibody-dependent cell-mediated cytotoxicity (ADCC) or phagocytosis (ADCP).

Fc positions that may be mutated to reduce binding of the protein to the activating FcγR and subsequently to reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in proteins with reduced ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

In some embodiments, the antibody or antigen binding fragment thereof that binds VEGFR1 is conjugated to an IgG1 heavy chain constant region or a fragment of the IgG1 heavy chain constant region. In some embodiments, the IgG1 heavy chain constant region comprises at least one mutation that results in reduced binding of the antibody to a FcγR. In some embodiments, the at least one mutation that results in reduced binding of the antibody to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index. In some embodiments, the first Ig constant region or the fragment of the first Ig constant region and/or the second Ig constant region or the fragment of the second Ig constant region comprise the following mutations: L234A_L235A_D265S. In some embodiments, the FcγR is FcγRI, FcγRIIA, FcγRIIB or FcγRIII, or any combination thereof.

In some embodiments, the antibody or antigen binding fragment of the disclosure and their function equivalents is conjugated to an Ig constant region or to a fragment of an Ig constant region comprising at least one mutation in the Fc region that enhances binding of the protein to an Fcγ receptor (FcγR) and/or enhances Fc effector functions such as Clq binding, complement dependent cytotoxicity (CDC), antibody-dependent cell-mediated cytotoxicity (ADCC) and/or phagocytosis (ADCP).

Fc positions that may be mutated to increase binding of the protein to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index). Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination mutations that result in proteins with increased ADCC or ADCP are a S239D/1332E, S298A/E333A/K334A, F243L/R292P/Y300L, F243L/R292P/Y300L/P396L, F243L/R292P/Y300L/V305I/P396L and G236A/S239D/1332E.

Fc positions that may be mutated to enhance CDC include positions 267, 268, 324, 326, 333, 345 and 430. Exemplary mutations that may be made singularly or in combination are S267E, F1268F, S324T, K326A, K326W, E333A, E345K, E345Q, E345R, E345Y, E430S, E430F and E430T. Exemplary combination mutations that result in proteins with increased CDC are K326A/E333A, K326W/E333A, H268F/S324T, S267E/H268F, S267E/S324T and S267E/H268F/S324T.

In some embodiments, the disclosure provides an isolated antibody or antigen binding fragment thereof comprising a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the disclosure provides an isolated antibody or antigen binding fragment thereof comprising a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH which is at least 80% (e.g. at least 85%, at least 90%, at least 95%, or at least 99%) identical to the VH of SEQ ID NO: 39 and a VL of at least 80% (e.g. at least 85%, at least 90%, at least 95%, or at least 99%) identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

The disclosure also provides an isolated antibody or antigen binding fragment thereof comprising a VH which is at least 80% (e.g. at least 85%, at least 90%, at least 95%, at least 99% or 100%) identical to the VH of SEQ ID NO: 39 and a VL which is at least 80% (e.g. at least 85%, at least 90%, at least 95%, at least 99% or 100%) identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH which is at least 80% (e.g. at least 85%, at least 90%, at least 95% or at least 99%) identical to the VH of SEQ ID NO: 39 and a VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH of SEQ ID NO: 39 and a VL which is at least 80% (e.g. at least 85%, at least 90%, at least 95% or at least 99%) identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH which is at least 95% identical to the VH of SEQ ID NO: 39 and a VL which is at least 95% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH which is at least 95% identical to the VH of SEQ ID NO: 39 and a VL which is at least 99% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH which is at least 99% identical to the VH of SEQ ID NO: 39 and a VL which is at least 99% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a VH which is at least 99% identical to the VH of SEQ ID NO: 39 and a VL which is at least 95% identical to the VL of SEQ ID NO: 40, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiment, the disclosure also provides an isolated antibody or antigen binding fragment thereof comprising an amino acid sequence at least 80% (e.g. at least 85%, at least 90%, at least 95%, at least 99% or 100%) identical to the amino acid sequence of SEQ ID NO: 59 and wherein the antibody or antigen binding fragment thereof binds VEGFR1, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the disclosure also provides an isolated antibody or antigen binding fragment thereof comprising an amino acid sequence at least 80% (e.g. at least 85%, at least 90%, at least 95%, at least 99% or 100%) identical to the amino acid sequence of SEQ ID NO: 60; and wherein the antibody or antigen binding fragment thereof binds VEGFR1, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

The disclosure also provides an isolated antibody or antigen binding fragment thereof comprising a HC which is at least 80% (e.g. at least 85%, at least 90%, at least 95%, at least 99% or 100%) identical to the HC of SEQ ID NO: 59 and a LC which is at least 80% (e.g. at least 85%, at least 90%, at least 95%, at least 99% or 100%) identical to the LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 80% (e.g. at least 85%, at least 90%, at least 95% or at least 99%) identical to the HC of SEQ ID NO: 59 and a LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC of SEQ ID NO: 59 and a LC which is at least 80% (e.g. at least 85%, at least 90%, at least 95% or at least 99%) identical to the LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 95% identical to the HC of SEQ ID NO: 59 and a LC which is at least 95% identical to the LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 95% identical to the HC of SEQ ID NO: 59 and a LC which is at least 99% identical to the LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 99% identical to the HC of SEQ ID NO: 59 and a LC which is at least 99% identical to the LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 99% identical to the HC of SEQ ID NO: 59 and a LC which is at least 95% identical to the LC of SEQ ID NO: 60, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

The disclosure also provides an isolated antibody or antigen binding fragment thereof comprising a HC which is at least 80% (e.g. at least 85%, at least 90%, at least 95%, at least 99% or 100%) identical to the HC of SEQ ID NO: 59 and a LC which is at least 80% (e.g. at least 85%, at least 90%, at least 95%, at least 99% or 100%) identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 80% (e.g. at least 85%, at least 90%, at least 95% or at least 99%) identical to the HC of SEQ ID NO: 59 and a LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC of SEQ ID NO: 59 and a LC which is at least 80% (e.g. at least 85%, at least 90%, at least 95% or at least 99%) identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 95% identical to the HC of SEQ ID NO: 59 and a LC which is at least 95% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 95% identical to the HC of SEQ ID NO: 59 and a LC which is at least 99% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 99% identical to the HC of SEQ ID NO: 59 and a LC which is at least 99% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 99% identical to the HC of SEQ ID NO: 59 and a LC which is at least 95% identical to the LC of SEQ ID NO: 60, wherein the antibody or antigen binding fragment comprises a HCDR1, a HCDR2, a HCDR3, a LCDR1, a LCDR2 and a LCDR3 of SEQ ID NOs: 25, 26, 27, 28, 29, and 30, respectively, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

The disclosure also provides an isolated antibody or antigen binding fragment thereof comprising a HC which is at least 80% (e.g. at least 85%, at least 90%, at least 95%, at least 99% or 100%) identical to the HC of SEQ ID NO: 59 and a LC which is at least 80% (e.g. at least 85%, at least 90%, at least 95%, at least 99% or 100%) identical to the LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 80% (e.g. at least 85%, at least 90%, at least 95% or at least 99%) identical to the HC of SEQ ID NO: 59 and a LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC of SEQ ID NO: 59 and a LC which is at least 80% (e.g. at least 85%, at least 90%, at least 95% or at least 99%) identical to the LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 95% identical to the HC of SEQ ID NO: 59 and a LC which is at least 95% identical to the LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 95% identical to the HC of SEQ ID NO: 59 and a LC which is at least 99% identical to the LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 99% identical to the HC of SEQ ID NO: 59 and a LC which is at least 99% identical to the LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

In some embodiments, the antibody or antigen binding fragment thereof that binds VEGFR1 comprises a HC which is at least 99% identical to the HC of SEQ ID NO: 59 and a LC which is at least 95% identical to the LC of SEQ ID NO: 60, wherein the antibody or antigen binding fragment comprises a VH of SEQ ID NO: 39 and a VL of SEQ ID NO: 40, and wherein the antibody or antigen binding fragment is IgG1 (e.g. IgG1λ), optionally wherein the first Ig constant region or the fragment of the first Ig constant region and/or the second Ig constant region or the fragment of the second Ig constant region comprise the following mutations: L234A_L235A_D265S, such as wherein the first Ig constant region or the fragment of the first Ig constant region and the second Ig constant region or the fragment of the second Ig constant region comprise the following mutations: L234A_L235A_D265S.

### Polynucleotides

Polynucleotides encoding the anti-VEGFR1 antibody or antigen binding fragment of the disclosure and their functional equivalents are also provided. The disclosure provides an isolated polynucleotide encoding any of the anti-VEGFR1 antibody or antigen binding fragment thereof of the disclosure.

In some embodiment, the disclosure provides an isolated polynucleotide encoding the VH of SEQ ID NO: 31.

In some embodiment, the disclosure provides an isolated polynucleotide encoding the VH of SEQ ID NO: 33.

In some embodiment, the disclosure provides an isolated polynucleotide encoding the VH of SEQ ID NO: 34.

In some embodiment, the disclosure provides an isolated polynucleotide encoding the VH of SEQ ID NO: 36.

In some embodiment, the disclosure provides an isolated polynucleotide encoding the VH of SEQ ID NO: 38.

In some embodiment, the disclosure provides an isolated polynucleotide encoding the VH of SEQ ID NO: 39.

In some embodiment, the disclosure provides an isolated polynucleotide encoding the VL of SEQ ID NO: 32.

In some embodiment, the disclosure provides an isolated polynucleotide encoding the VL of SEQ ID NO: 35.

In some embodiment, the disclosure provides an isolated polynucleotide encoding the VL of SEQ ID NO: 37.

In some embodiment, the disclosure provides an isolated polynucleotide encoding the VL of SEQ ID NO: 40.

In some embodiment, the disclosure provides an isolated polynucleotide encoding the heavy chain of SEQ ID NO: 51.

In some embodiment, the disclosure provides an isolated polynucleotide encoding the heavy chain of SEQ ID NO: 53.

In some embodiment, the disclosure provides an isolated polynucleotide encoding the heavy chain of SEQ ID NO: 54.

In some embodiment, the disclosure provides an isolated polynucleotide encoding the heavy chain of SEQ ID NO: 56.

In some embodiment, the disclosure provides an isolated polynucleotide encoding the heavy chain of SEQ ID NO: 58.

In some embodiment, the disclosure provides an isolated polynucleotide encoding the heavy chain of SEQ ID NO: 59.

In some embodiment, the disclosure provides an isolated polynucleotide encoding the light chain of SEQ ID NO: 52.

In some embodiment, the disclosure provides an isolated polynucleotide encoding the light chain of SEQ ID NO: 55.

In some embodiment, the disclosure provides an isolated polynucleotide encoding the light chain of SEQ ID NO: 57.

In some embodiment, the disclosure provides an isolated polynucleotide encoding the light chain of SEQ ID NO: 60.

In some embodiments, the disclosure provides isolated polynucleotide sequences encoding polypeptide sequences of SEQ ID NOs: 59 and 60.

In some embodiments, the disclosure provides an isolated polynucleotide of SEQ ID NO: 41, 42, 43, 44, 45, 46, 47, 48, 49, 50, 61, 62, 63, 64, 65, 66, 67, 68, 69 or 70.

In some embodiments, the disclosure provides an isolated polynucleotide comprising a polynucleotide sequence at least 80%, such as at least 85%, at least 90%, at least 95%, at least 99% or 100%, identical to the polynucleotide sequence of SEQ ID NOs: 41, 42, 43, 44, 45, 46, 47, 48, 49, 50, 61, 62, 63, 64, 65, 66, 67, 68, 69, or 70.

Polynucleotides encoding the anti-VEGFR1 antibody or antigen binding fragment of the disclosure include polynucleotides with nucleic acid sequences that are substantially the same as the nucleic acid sequences of the polynucleotide of the disclosure. “Substantially the same” nucleic acid sequence is defined herein as a sequence with at least 80% identity to another nucleic acid sequence when the two sequences are aligned. Two nucleic acid sequences are substantially identical if the polypeptide encoded by the first nucleic acid is immunologically cross reactive with the polypeptide encoded by the second nucleic acid. Another indication that two nucleic acid sequences are substantially identical is that the two molecules hybridize to each other under stringent conditions.

Modified nucleotides may be used to generate the polynucleotides of the disclosure. Exemplary modified nucleotides are 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N⁶-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N⁶-isopentenyladenine, uracil-5-oxyacetic acid (v), wybutoxosine, pseudouracil, queuosine, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

### Vectors Comprising Polynucleotides Encoding for the Anti-VEGFR1 Antibodies

Vectors comprising DNA encoding the anti-VEGFR1 antibody or antigen binding fragment of the disclosure are also provided. The disclosed vectors can be used, for example, to generate any of the above disclosed anti-VEGFR1 antibody, or antigen binding fragment thereof. Polynucleotides encoding any of the anti-VEGFR1 antibody or antigen binding fragment thereof of the disclosure may be incorporated into vectors using standard molecular biology methods.

In some embodiments, the disclosure provides an expression vector comprising the polynucleotide of the invention. Such vectors may be plasmid vectors, viral vectors, vectors for baculovirus expression, transposon-based vectors or any other vector suitable for introduction of the synthetic polynucleotide of the invention into a given organism or genetic background by any means. The vector of the disclosure may be an expression vector for the efficient synthesis of VEGFR1 antibody polypeptide and expression of the VEGFR1 antibody polypeptide of the disclosure in prokaryotic and eukaryotic systems, including but not limited to yeast and mammalian cell culture.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λEMBL4, and λNM1149, λZapII (Stratagene) can be used. Exemplary plant expression vectors include pBI01, pBI01.2, pBI121, pBI101.3, and pBIN19 (Clontech). Exemplary animal expression vectors include pEUK-Cl, pMAM, and pMAMneo (Clontech). The expression vector may be a viral vector, e.g., a retroviral vector, e.g., a gamma retroviral vector.

The vector of the disclosure may contain a promoter and an enhancer sequence. Polynucleotides encoding the VEGFR1 binding proteins of the disclosure may be operably linked to control sequences in the expression vector(s) that ensure the expression of the VEGFR1 binding proteins. Such regulatory elements may include a transcriptional promoter, sequences encoding suitable mRNA ribosomal binding sites, and sequences that control the termination of transcription and translation. Expression vectors may also include one or more non-transcribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

Vectors of the disclosure may also contain one or more Internal Ribosome Entry Site(s) (IRES). Inclusion of an IRES sequence into fusion vectors may be beneficial for enhancing expression of some proteins. In some embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColE1, SV40, 2μ plasmid, λ, bovine papilloma virus, and the like.

The recombinant expression vectors can be designed for either transient expression, for stable expression, or for both. Also, the recombinant expression vectors can be made for constitutive expression or for inducible expression.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol×resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

### Host Cells

The disclosure also provides for a host cell comprising any of the vectors of the disclosure. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-KISV (Lonza Biologics, Walkersville, MD), CHO-K1 (ATCC CRL-61) or DG44.

The disclosure provides recombinant host cells containing any of the expression vectors of the disclosure. Nucleic acids encoding any of the VEGFR1 binding proteins or fragments thereof can be used for transformation of a suitable mammalian host cell. Host cell transformation, culture, antibody expression and purification are done using well known methods.

Cell lines may be selected based on high level of expression of the VEGFR1 antibody of interest and minimal contamination from host cell proteins. Mammalian cell lines available as host cells for expression are well known in the art and include, but are not limited to from Chinese Hamster Ovary (CHO) cells such as CHO-KISV (Lonza Biologics, Walkersville, MD), CHO-K1 (ATCC CRL-61), or CHO DG44, and Baby Hamster Kidney (BHK) cells. These cell lines can be used to produce any of the anti-VEGFR1 antibody or antibody fragment of the disclosure by culturing the cells under conditions suitable for expression of the antibody and purifying the antibody from the host cell or medium surrounding the host cell.

The disclosure also provides a method of producing the anti-VEGFR1 binding protein of the disclosure comprising culturing the host cell of the disclosure in conditions that the anti-VEGFR1 binding protein is expressed, and recovering the anti-VEGFR1 antibody binding protein produced by the host cell using well known methods in the art. A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

In some embodiments, the disclosure provides a host cell expressing any of the isolated antibodies or antigen binding fragment thereof of the present disclosure.

In some embodiments, the disclosure provides a host cell comprising the vector of the present disclosure.

### Pharmaceutical Composition

Also provided is the use of any of the disclosed antibodies for the preparation of a medicament for treating a chronic kidney disease.

Also provided is the use of any of the disclosed antibodies for the preparation of a pharmaceutical composition for treating a chronic kidney disease.

Also provided is a pharmaceutical composition comprising the antibody or antigen binding fragment thereof of the disclosure and a pharmaceutically acceptable carrier.

For therapeutic use, the anti-VEGFR1 antibody and antigen binding fragment thereof of the disclosure may be prepared as pharmaceutical compositions containing an effective amount of the antibody as an active ingredient in a pharmaceutically acceptable carrier.

“Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies of the invention in such pharmaceutical formulation may vary from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

A pharmaceutically acceptable carrier can include a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Pharmaceutical compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Pharmaceutical compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

### Method of Treatment and Uses

Also provided is the use of any of the disclosed antibody, antigen binding fragment thereof or pharmaceutical composition for treating a chronic kidney disease.

“Treat,” “treating,” or “treatment” of a disease or disorder such as chronic kidney disease refers to accomplishing one or more of the following: reducing the severity and/or duration of the disorder, delaying the progression of the disorder, slowing the progression of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder. As used herein, the terms “delaying the progression of” or “slowing the progression of” shall include (a) delaying or slowing the development of one or more symptoms or complications of the disease, condition or disorder; (b) delaying or slowing the development of one or more new/additional symptoms or complications of the disease, condition or disorder; and/or (c) delaying or slowing the progression of the disease, condition or disorder to a later stage or more serious form of said disease, condition or disorder.

“Subject” includes any human or nonhuman animal. “Nonhuman animal” includes all vertebrates, e.g., mammals and non-mammals. The term “mammal” as used herein, encompasses any mammal. Examples of mammals include, but are not limited to, non-human primates, cows, horses, sheep, pigs, cats, dogs, mice, rats, rabbits, guinea pigs, monkeys, humans, etc. The terms “subject” and “patient” can be used interchangeably herein. In some embodiments, the subject or patient is human.

An isolated antibody or antigen fragment thereof of the application can prevent the binding of VEGFA to a VEGFR1 in a subject in need thereof. Thus, another general aspect of the application relates to a method of preventing the binding of VEGFA to a VEGFR1 in a subject in need thereof, comprising administering to the subject an effective amount of an isolated antibody or antigen fragment thereof, an immunoconjugate, a pharmaceutical composition, an isolated polynucleotide, a vector, or a host cell of the application to thereby prevent the binding of VEGFA to the VEGFR1. The subject can be in need of a treatment of a disease, disorder or a medical condition related to the binding of VEGFA to a VEGFR1.

In some embodiments, the subject is in need of a treatment of a Chronic Kidney Disease (CKD), in need of decreasing proteinuria, has advanced stage 4 or 5 chronic kidney disease, or has CKD with proteinuria, albuminuria, or diabetes mellitus.

In some embodiments, a method of preventing the binding of VEGFA to a VEGFR1 in a subject in need thereof, comprising administering to the subject an effective amount of any of the isolated antibody or antigen fragment thereof of the disclosure, any of the the immunoconjugate of the disclosure, any of the pharmaceutical composition of the disclosure, any of the isolated polynucleotide of the disclosure, any of the vector of disclosure or any of the host cell of the disclosure to thereby prevent the binding of VEGFA to the VEGFR1.

Also provided are methods of treating a medical condition by administering a therapeutically effective amount of an anti-VEGFR1 antibody or antigen binding fragment thereof to a subject in need thereof. In some embodiments, the medical condition is chronic kidney disease. A “therapeutically effective amount” refers to an amount effective, at doses and for periods of time necessary, to achieve a desired therapeutic result. A therapeutically effective amount may vary depending on factors such as the disease state, age, sex, and weight of the individual.

In some embodiments, the medical condition is chronic kidney disease. In some embodiments, the disclosure provides a method of treating chronic kidney disease in a subject, comprising administering to the subject a therapeutically effective amount of the disclosed pharmaceutical compositions.

“Chronic kidney disease” is meant to include all types of chronic kidney disease (CKD) of any etiology or caused by any type of conditions associated with chronic kidney disease, and generating all types of symptoms associated with chronic kidney disease. CKDs include Primary Glomerular Disease (including but not limited to Nephropathy and focal segmental glomerular sclerosis), Secondary Glomerular Disease (including but not limited to lupus nephritis), Thrombotic Microangiopathy, Tubulointerstitial Diseases, Ischemic Nephropathy, Diabetic Nephropathy, Polycystic Kidney Disease, Hypertensive Nephropathy, Focal Segmental Glomerulosclerosis, Nephrotic Syndrome, and Obstructive Uropathy (including but not limited to reflux nephropathy).

In some embodiments, the disclosure provides a method of treating diabetic kidney disease in a subject, comprising administering to the subject in need thereof a therapeutically effective amount of any of the anti-VEGFR1 antibody or antigen binding fragment thereof of the disclosure or any of the disclosed pharmaceutical compositions of the disclosure.

In some embodiments, the disclosure provides a method of treating chronic kidney disease in a subject, comprising administering to a subject in need thereof a therapeutically effective amount of a anti-VEGFR1 antibody or antigen binding fragment thereof of the disclosure, wherein the chronic kidney disease is diabetic nephropathy.

In some embodiments, the disclosure provides a method of delaying the progression or slowing the progression of chronic kidney disease in a subject, comprising administering to a subject in need thereof a therapeutically effective amount of a anti-VEGFR1 antibody or antigen binding fragment thereof of the disclosure, wherein the chronic kidney disease is diabetic nephropathy.

In some embodiments, the disclosure provides a method of treating chronic kidney disease in a subject, comprising administering to a subject in need thereof a therapeutically effective amount of an anti-VEGFR1 antibody of the disclosure, wherein the chronic kidney disease is focal segmental glomerulosclerosis.

In some embodiments, the disclosure provides a method of delaying the progression or slowing the progression of chronic kidney disease in a subject, comprising administering to a subject in need thereof a therapeutically effective amount of an anti-VEGFR1 antibody of the disclosure, wherein the chronic kidney disease is focal segmental glomerulosclerosis.

In some embodiments, the disclosure provides a method of treating chronic kidney disease in a subject, comprising administering to a subject in need thereof a therapeutically effective amount of anti-VEGFR1 antibody or antigen binding fragment thereof of the disclosure, wherein the chronic kidney disease is hypertensive nephropathy.

In some embodiments, the disclosure provides a method of delaying the progression or slowing the progression of chronic kidney disease in a subject, comprising administering to a subject in need thereof a therapeutically effective amount of anti-VEGFR1 antibody or antigen binding fragment thereof of the disclosure, wherein the chronic kidney disease is hypertensive nephropathy.

In some embodiments, the subject has chroninc kidney disease (CKD). Chronic kidney disease has been classified into five stages of kidney disease. Stages of kidney disease are clasified based on the rate the kidney is able to filter out waste and extra fluid from the blood. The estimated glomerular filtration rate (eGFR) number is based on levels of creatine and waste product in the blood and has been used to classify the five stages of kidney damage, from very mild damage in stage 1 to kidney failure in stage 5.

In some embodiments, the method of treating a Chronic Kidney Disease (CKD) in a subject in need thereof, comprises administering a therapeutically effective amount of any of the isolated antibody or antigen fragment thereof of the disclosure, any of the immunoconjugate of the disclosure, any of the pharmaceutical composition of the disclosure, any of the isolated polynucleotide of the disclosure, any of the vector of the disclosure, or any of the host cell of the disclosure to the subject for a time sufficient to treat the CKD.

In some embodiments, the disclosure provides a VEGFR1 antibody for use in decreasing the loss of glomerular filtration rate (GFR).

In some embodiments, the disclosure provides a method for decreasing the loss of glomerular filtration rate (GFR), comprising administering to a subject in need thereof a therapeutically effective amount of the anti-VEGFR1 antibody or antigen binding fragment of the disclosure.

In some embodiments, the subject has advanced stage 3, 4 or 5 chronic kidney disease with an eGFR (estimated glomerular filtration rate) below 60 ml/min/1.73 m². In some embodiments, the subject has advanced stage 4 or 5 chronic kidney disease with a measured eGFR equal to or lower than 30 ml/min/1.73 m². In some embodiments, the subject has stage 3 chronic kidney disease with a measured eGFR between 30 and 60 ml/min/1.73 m². In some embodiments, the subject has stage 3b chronic kidney disease with a measured eGFR between 30 and 45 ml/min/1.73 m². In some embodiments, the subject has advanced stage 4 or 5 chronic kidney disease and is not on dialysis. In some embodiments, the subject has advanced stage 4 or 5 chronic kidney disease and is on dialysis.

In some embodiments, the disclosure provides a method of treating chronic kidney disease in a subject, comprising administering to a subject in need thereof a therapeutically effective amount of an anti-VEGFR1 antibody or antigen binding fragment thereof of the disclosure, wherein the subject has stage 4 or 5 chronic kidney disease with a measured eGFR equal or below 30 ml/min/1.73 m².

In some embodiments, the disclosure provides a method of delaying the progression or slowing the progression of chronic kidney disease in a subject, comprising administering to a subject in need thereof a therapeutically effective amount of an anti-VEGFR1 antibody or antigen binding fragment thereof of the disclosure, wherein the subject has stage 4 or 5 chronic kidney disease with a measured eGFR equal or below 30 ml/min/1.73 m².

In some embodiments, the disclosure provides a method of treating chronic kidney disease in a subject, comprising administering to a subject in need thereof a therapeutically effective amount of an anti-VEGFR1 antibody or antigen binding fragment thereof of the disclosure, wherein the subject has stage 3 chronic kidney disease with a measured eGFR between 30 and 60 ml/min/1.73 m².

In some embodiments, the disclosure provides a method of delaying the progression or slowing the progression of chronic kidney disease in a subject, comprising administering to a subject in need thereof a therapeutically effective amount of an anti-VEGFR1 antibody or antigen binding fragment thereof of the disclosure, wherein the subject has stage 3 chronic kidney disease with a measured eGFR between 30 and 60 ml/min/1.73 m².

In some embodiments, the subject has chronic kidney disease with proteinuria. In some embodiments, the subject has chronic kidney disease with albuminuria. In some embodiments, the subject has chronic kidney disease with diabetes mellitus.

In some embodiments, the disclosure provides a method of treating chronic kidney disease in a subject, comprising administering to a subject in need thereof a therapeutically effective amount of an anti-VEGFR1 antibody or antigen binding fragment thereof of the disclosure, wherein the subject has chronic kidney disease with proteinuria.

In some embodiments, the disclosure provides a method of delaying the progression or slowing the progression of chronic kidney disease in a subject, comprising administering to a subject in need thereof a therapeutically effective amount of an anti-VEGFR1 antibody or antigen binding fragment thereof of the disclosure, wherein the subject has chronic kidney disease with proteinuria.

In some embodiments, the disclosure provides a method of decreasing proteinuria in a subject in need thereof, comprising administering a therapeutically effective amount of any of the isolated antibody or antigen fragment thereof of the disclosure, any of the immunoconjugate of the disclosure, any of the pharmaceutical composition of the disclosure, any of the isolated polynucleotide of the disclosure, any of the vector of the disclosure, or any of the host cell of the disclosure, to the subject for a time sufficient to decrease proteinuria in the subject.

In some embodiments, the disclosure provides a method of treating chronic kidney disease in a subject, comprising administering to a subject in need thereof a therapeutically effective amount of an anti-VEGFR1 antibody or antigen binding fragment thereof of the disclosure, wherein the subject has chronic kidney disease with albuminuria.

In some embodiments, the disclosure provides a method of delaying the progression or slowing the progression of chronic kidney disease in a subject, comprising administering to a subject in need thereof a therapeutically effective amount of an anti-VEGFR1 antibody or antigen binding fragment thereof of the disclosure, wherein the subject has chronic kidney disease with albuminuria.

In some embodiments, the disclosure provides a method of treating chronic kidney disease in a subject, comprising administering to a subject in need thereof a therapeutically effective amount of an anti-VEGFR1 antibody or antigen binding fragment thereof of the disclosure, wherein the subject has chronic kidney disease with diabetes mellitus.

In some embodiments, the disclosure provides a method of delaying the progression or slowing the progression of chronic kidney disease in a subject, comprising administering to a subject in need thereof a therapeutically effective amount of an anti-VEGFR1 antibody or antigen binding fragment thereof of the disclosure, wherein the subject has chronic kidney disease with diabetes mellitus.

“Proteinuria” refers to the presence of increased amounts of protein in the urine. Proteinuria may reflect abnormal loss of plasma proteins due to a) increased glomerular permeability to large molecular weight proteins (albuminuria or glomerular proteinuria), b) incomplete tubular reabsorption of normally filtered low-molecular-weight proteins (tubular proteinuria), or c) increased plasma concentration of low-molecular-weight proteins (overproduction proteinuria, such as immunoglobulin light chains). Proteinuria may also reflect abnormal loss of proteins derived from the kidney (renal tubular cell constituents due to tubular damage) and lower urinary tract.

“Albuminuria” is a condition, where albumin is present in the urine. In healthy individuals, albumin is filtered by the kidneys. When the kidneys do not properly filter large molecules such as albumin from the urine, albumin is excreted in urine and is typically a sign of kidney damage. Albuminuria is a common but not uniform condition in CKD patients. It is the earliest marker of glomerular diseases, including diabetic glomerulosclerosis, where it generally appears before the reduction in eGFR. It is a marker of hypertensive nephrosclerosis but may not appear until after the reduction in eGFR.

“Diabetic nephropathy” is one of the microvascular complications of diabetes mellitus ad is characterized by persistent albuminuria and a progressive decline in renal function.

In some embodiments, the disclosure provides a method of decreasing proteinuria in a patient having chronic kidney disease, comprising administering to a patient in need thereof a therapeutically effective amount of the anti-VEGFR1 antibody or antigen binding fragment thereof of the disclosure.

In some embodiments, the disclosure provides a method of decreasing proteinuria in a patient in need thereof, comprising administering to said patient a therapeutically effective amount of the anti-VEGFR1 antibody or antigen binding fragment of the disclosure. In some embodiments, proteinuria is caused by chronic kidney disease. In other embodiments, proteinuria is caused by diabetic nephropathy.

In some embodiments, the disclosure provides a method of decreasing albuminuria in a patient having chronic kidney disease, comprising administering to a patient in need thereof a therapeutically effective amount of the anti-VEGFR1 antibody or antigen binding fragment thereof of the disclosure.

Also provided are methods of detecting VEGFR1 in a sample, comprising obtaining the sample, contacting the sample with an anti-VEGFR1 antibody or antigen binding fragment thereof of the disclosure and detecting the bound VEGFR1 in the sample.

In some embodiments, the sample may be derived from urine, blood, serum, plasma, saliva, ascites, circulating cells, synovial fluid, circulating cells, cells that are not tissue associated (i.e., free cells), tissues (e.g., surgically resected tissue, biopsies, including fine needle aspiration), histological preparations, and the like. VEGFR1 may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antibody and antigen binding fragment thereof of the disclosure may be used in a variety of assays to detect VEGFR1 in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

### Kits

Described herein are kits comprising the antibody or antigen binding fragment of the disclosure or their functional equivalents.

The disclosure provides a kit comprising the antibody or antigen binding fragment thereof that binds VEGFR1.

The kit may be used for therapeutic uses and as diagnostic kits.

The kit may be used to detect the presence of VEGFR1 in a sample.

In some embodiments, the kit comprises the anti-VEGFR1 antibody or antigen binding fragment of the disclosure and reagents for detecting the VEGFR1 binding protein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antibody or antigen binding fragment thereof that binds VEGFR1 in a container and instructions for use of the kit.

In some embodiments, the disclosure comprises a kit comprising any of the isolated antibody or antigen binding fragment of the disclosure, any of the immunoconjugate of the disclosure, any of the pharmaceutical composition of the disclosure, any of the isolated polynucleotide of the disclosure, any of the vector of the disclosure, any of the host cell of the disclosure.

In some embodiments, the antibody or antigen binding fragment thereof that binds VEGFR1 in the kit is labeled.

In some embodiments, the kit comprises the antibody or antigen binding fragment thereof that binds VEGFR1 comprising:
the VH of SEQ ID NO: 31 and the VL of SEQ ID NO: 32;
the VH of SEQ ID NO: 33 and the VL of SEQ ID NO: 32;
the VH of SEQ ID NO: 34 and the VL of SEQ ID NO: 35;
the VH of SEQ ID NO: 36 and the VL of SEQ ID NO: 37;
the VH of SEQ ID NO: 38 and the VL of SEQ ID NO: 37; or
the VH of SEQ ID NO: 39 and the VL of SEQ ID NO: 40.

In some embodiments, the kit comprises the antibody or antigen binding fragment thereof that binds VEGFR1 comprising a VH of SEQ ID NO: 39 and a VL of SEQ ID NO: 40.

In some embodiments, the kit comprises the antibody or antigen binding fragment thereof that binds VEGFR1 comprising the amino acid sequence of SEQ ID NOs: 51, 52, 53, 54, 55, 56, 57, 59 or 60.

In some embodiments, the kit comprises antibody or antigen binding fragment thereof that binds VEGFR1 comprising the amino acid sequence selected from the group consisting of (a) SEQ ID NO: 51 and SEQ ID NO: 52; (b) SEQ ID NO: 53 and SEQ ID NO: 52; (c) SEQ ID NO: 54 and SEQ ID NO: 55; (d) SEQ ID NO: 56 and SEQ ID NO: 57; (e) SEQ ID NO: 58 and SEQ ID NO: 57; and SEQ ID NOs: 59 and SEQ ID NO: 60.

### EMBODIMENTS

The following list of embodiments is intended to compliment, rather than displace or supersede, the previous descriptions.

Embodiment 1. An isolated antibody or antigen binding fragment thereof that binds to an epitope within the amino acid sequence of SEQ ID NO: 173 or SEQ ID NO: 4, and wherein the antibody or antigen binding fragment thereof prevents the binding of VEGFA to VEGFR1.

Embodiment 2. An isolated antibody or antigen binding fragment thereof that binds to an epitope within the amino acid sequence of SEQ ID NO: 173, and wherein the antibody or antigen binding fragment thereof prevents the binding of VEGFA to VEGFR1.

Embodiment 3. An isolated antibody or antigen binding fragment thereof that binds to an epitope within the amino acid sequence of SEQ ID NO: 4, and wherein the antibody or antigen binding fragment thereof prevents the binding of VEGFA to VEGFR1.

Embodiment 4. The isolated antibody or antigen binding fragment thereof of any one of embodiments 1-3, wherein the antibody or antigen binding fragment thereof binds to epitopes on VEGFR1 having the amino acid sequence FPLDTL (SEQ ID NO: 143) or EIGL (SEQ ID NO: 144) or binds to the epitopes having the amino acid sequences FPLDTL (SEQ ID NO: 143) and EIGL (SEQ ID NO: 144).

Embodiment 5. The isolated antibody or antigen binding fragment thereof of embodiment 4, wherein the antibody or antigen binding fragment thereof binds to epitopes on VEGFR1 having the amino acid sequence FPLDTL (SEQ ID NO: 143).

Embodiment 6. The isolated antibody or antigen binding fragment thereof of embodiment 4, wherein the antibody or antigen binding fragment thereof binds to epitopes on VEGFR1 having the amino acid sequence EIGL (SEQ ID NO: 144).

Embodiment 7. The isolated antibody or antigen binding fragment thereof of embodiment 4 wherein the antibody or antigen binding fragment thereof binds to human, mouse, rat and/or cynomolgus monkey VEGFR1.

Embodiment 8. The isolated antibody or antigen binding fragment thereof of embodiment 7, wherein the antibody or antigen binding fragment thereof binds human VEGFR1.

Embodiment 9. The isolated antibody or antigen binding fragment thereof of embodiment 8, wherein the antibody or antigen binding fragment thereof binds to human VEGFR1 and a VEGFR1 from at least one species selected from the group consisting of cynomolgus monkey, mouse and rat.

Embodiment 10. The isolated antibody or antigen binding fragment thereof of embodiment 9, wherein the antibody or antigen binding fragment thereof binds to human and mouse VEGFR1.

Embodiment 11. The isolated antibody or antigen binding fragment thereof of embodiment 9, wherein the antibody or antigen binding fragment thereof binds to human and rat VEGFR1.

Embodiment 12. The isolated antibody or antigen binding fragment thereof of embodiment 9, wherein the antibody or antigen binding fragment thereof binds to human, mouse and rat VEGFR1.

Embodiment 13. The isolated antibody or antigen binding fragment thereof of embodiment 9, wherein the antibody or antigen binding fragment thereof binds to human, and cynomolgus monkey VEGFR1.

Embodiment 14. The isolated antibody or antigen binding fragment thereof of embodiment 9, wherein the antibody or antigen binding fragment thereof binds to human, mouse, rat and cynomolgus monkey VEGFR1.

Embodiment 15. The isolated antibody or antigen binding fragment thereof of embodiment 9, wherein the antibody or antigen binding fragment thereof binds to a human VEGFR1, mouse VEGFR1, rat VEGFR1 and cynomolgus monkey VEGFR1, with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

Embodiment 16. The isolated antibody or antigen binding fragment thereof of embodiment 9, wherein the antibody or antigen binding fragment thereof binds to a human VEGFR1, mouse VEGFR1, and cynomolgus monkey VEGFR1, with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

Embodiment 17. The isolated antibody or antigen binding fragment thereof of embodiment 9, wherein the antibody or antigen binding fragment thereof binds to a human VEGFR1 and mouse VEGFR1 with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

Embodiment 18. The isolated antibody or antigen binding fragment thereof of embodiment 9, wherein the antibody or antigen binding fragment thereof binds to a human VEGFR1 and rat VEGFR1 with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

Embodiment 19. The isolated antibody or antigen binding fragment thereof of embodiment 9, wherein the antibody or antigen binding fragment thereof binds to a human VEGFR1 and cynomolgus monkey VEGFR1 with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

Embodiment 20. The isolated antibody or antigen binding fragment thereof of any one of embodiments 1-19, comprising the heavy chain complementarity determining regions (HCDRs) of a VH of SEQ ID NO: 31, 33, 34, 36, 38 or 39, and the light chain complementarity determining regions (LCDRs) of a VL of SEQ ID NO: 32, 35, 37 or 40.

Embodiment 21. The isolated antibody or antigen binding fragment thereof of embodiment 20, comprising:
a. the HCDRs of the VH having the amino acid sequence of SEQ ID NO: 31, and the LCDRs of the VL having the amino acid sequence of SEQ ID NO: 32;
b. the HCDRs of the VH having the amino acid sequence of SEQ ID NO: 33, and the LCDRs of the VL having the amino acid sequence of SEQ ID NO: 32;
c. the HCDRs of the VH having the amino acid sequence of SEQ ID NO: 34, and the LCDRs of the VL having the amino acid sequence of SEQ ID NO: 35;
d. the HCDRs of the VH having the amino acid sequence of SEQ ID NO: 36, and the LCDRs of the VL having the amino acid sequence of SEQ ID NO: 37;
e. the HCDRs of the VH having the amino acid sequence of SEQ ID NO: 38, and the LCDRs of the VL having the amino acid sequence of SEQ ID NO: 37; or
f. the HCDRs of the VH having the amino acid sequence of SEQ ID NO: 39, and the LCDRs of the VL having the amino acid sequence of SEQ ID NO: 40.

Embodiment 22. The isolated antibody or antigen binding fragment thereof of embodiment 21 comprising a VH having a HCDR1, a HCDR2 and a HCDR3, and a VL having a LCDR1, a LCDR2, and a LCDR3, wherein the HCDR1, HCDR2, HCDR3, LCDR1, LCDR2 and LCDR3 comprise the amino acid sequences of:
a. SEQ ID NOs: 7, 175, 9, 10, 11 and 12, respectively;
b. SEQ ID NOs: 7, 8, 9, 10, 11 and 12, respectively;
c. SEQ ID NOs: 13, 14, 15, 16, 17 and 18, respectively;
d. SEQ ID NOs: 19, 20, 21, 22, 23 and 24, respectively;
e. SEQ ID NOs: 25, 26, 27, 28, 29, and 30, respectively;
f. SEQ ID NOs: 71, 176, 73, 74, 75 and 76, respectively;
g. SEQ ID NOs: 71, 72, 73, 74, 75 and 76, respectively;
h. SEQ ID NOs: 77, 78, 79, 80, 81, and 82, respectively;
i. SEQ ID NOs: 83, 84, 85, 86, 87, and 88, respectively;
j. SEQ ID NOs: 89, 90, 91, 92, 93 and 94, respectively;
k. SEQ ID NOs: 95, 177, 97, 98, 99 and 100, respectively
l. SEQ ID NOs: 95, 96, 97, 98, 99 and 100, respectively;
m. SEQ ID NOs: 101, 102, 103, 104, 105 and 106, respectively;
n. SEQ ID NOs: 107, 108, 109, 110, 111, and 112, respectively;
o. SEQ ID NOs: 113, 114, 115, 116, 117, and 118, respectively;
p. SEQ ID NOs: 119, 178, 121, 122, amino acid sequence LNS, and SEQ ID NO: 124, respectively;
q. SEQ ID NOs: 119, 120, 121, 122, amino acid sequence LNS, and SEQ ID NO: 124, respectively;
r. SEQ ID NOs: 125, 126, 127, 128, amino acid sequence FNF, and SEQ ID NO: 130, respectively;
s. SEQ ID NOs: 131, 132, 133, 134, amino acid sequence YD, and SEQ ID NO: 136, respectively; or
t. SEQ ID NOs: 137, 138, 139, 140, amino acid sequence FNS, and SEQ ID NO: 142, respectively.

Embodiment 23. The isolated antibody or antigen binding fragment thereof of embodiment 22 wherein the HCDR1, HCDR2, HCDR3, LCDR1, LCDR2 and LCDR3 comprise the amino acid sequences of SEQ ID NOs: 7, 175, 9, 10, 11 and 12, respectively.

Embodiment 24. The isolated antibody or antigen binding fragment thereof of embodiment 22, wherein the HCDR1, HCDR2, HCDR3, LCDR1, LCDR2 and LCDR3 comprise the amino acid sequences of SEQ ID NOs: 13, 14, 15, 16, 17, and 18, respectively.

Embodiment 25. The isolated antibody or antigen binding fragment thereof of embodiment 22, wherein the HCDR1, HCDR2, HCDR3, LCDR1, LCDR2 and LCDR3 comprise the amino acid sequences of SEQ ID NOs: 25, 26, 27, 28, 29 and 30, respectively.

Embodiment 26. The isolated antibody or antigen binding fragment thereof of embodiment 22, wherein the HCDR1, HCDR2, HCDR3, LCDR1, LCDR2 and LCDR3 comprise the amino acid sequences of SEQ ID NOs: 7, 8, 9, 10, 11, and 12, respectively.

Embodiment 27. The isolated antibody or antigen binding fragment thereof of embodiment 22, wherein the HCDR1, HCDR2, HCDR3, LCDR1, LCDR2 and LCDR3 comprise the amino acid sequences of SEQ ID NOs: 19, 20, 21, 22, 23, and 24, respectively.

Embodiment 28. The isolated antibody or antigen binding fragment thereof of any one of embodiments 1-27, comprising a heavy chain variable domain (VH) having an amino acid sequence at least 80% (e.g. at least 85%, at least 90%, at least 95%, or at least 99%) identical to the amino acid sequence of SEQ ID NO: 31, 33, 34, 36, 38 or 39, and a light chain variable domain (VL) having an amino acid sequence at least 80% (e.g. at least 85%, at least 90%, at least 95%, or at least 99%) identical to the amino acid sequence of SEQ ID NO: 32, 35, 37 or 40.

Embodiment 29. The isolated antibody or antigen binding fragment thereof of embodiment 28, comprising a VH and a VL comprising the amino acid sequences of:
a. SEQ ID NOs: 31 and 32, respectively;
b. SEQ ID NOs: 33 and 32, respectively;
c. SEQ ID NOs: 34 and 35, respectively;
d. SEQ ID NOs: 36 and 37, respectively;
e. SEQ ID NOs: 38 and 37, respectively; or
f. SEQ ID NOs: 39 and 40, respectively.

Embodiment 30. The isolated antibody or antigen binding fragment thereof of embodiment 29, comprising the VH which is at least 80% (e.g. at least 85%, at least 90%, at least 95%, at least 99% or 100%) identical to the amino acid sequence of SEQ ID NO: 39, and the VL which is at least 80% (e.g. at least 85%, at least 90%, at least 95%, at least 99% or 100%) identical to the amino acid sequence of SEQ ID NO: 40.

Embodiment 31. The isolated antibody or antigen binding fragment thereof of embodiment 30, comprising the VH having the amino acid sequence of SEQ ID NO: 39 and the VL having the amino acid sequence of SEQ ID NO: 40.

Embodiment 32. The isolated antibody or antigen binding fragment thereof of embodiment 29, comprising the VH which is at least 80% (e.g. at least 85%, at least 90%, at least 95%, at least 99% or 100%) identical to the amino acid sequence of SEQ ID NO: 33 and a VL which is at least 80% (e.g. at least 85%, at least 90%, at least 95%, at least 99% or 100%) identical to the amino acid sequence of SEQ ID NO: 32.

Embodiment 33. The isolated antibody or antigen binding fragment thereof of embodiment 32, comprising the VH comprising the amino acid sequence of SEQ ID NO: 33 and the VL comprising the amino acid sequence of SEQ ID NO: 32.

Embodiment 34. The isolated antibody or antigen binding fragment thereof of embodiment 29, comprising the VH which is at least 80% (e.g. at least 85%, at least 90%, at least 95%, at least 99% or 100%) identical to the amino acid sequence of SEQ ID NO: 38 and the VL which is at least 80% (e.g. at least 85%, at least 90%, at least 95%, at least 99% or 100%) identical to the amino acid sequence of SEQ ID NO: 37.

Embodiment 35. The isolated antibody or antigen binding fragment thereof of Embodiment 34, comprising the VH comprising the amino acid sequence of SEQ ID NO: 38 and the VL comprising the amino acid sequence of SEQ ID NO: 37.

Embodiment 36. The isolated antibody or antigen binding fragment thereof of embodiment 29, comprising the VH which is at least 80% (e.g. at least 85%, at least 90%, at least 95%, at least 99% or 100%) identical to the amino acid sequence of SEQ ID NO: 31 and the VL which is at least 80% (e.g. at least 85%, at least 90%, at least 95%, at least 99% or 100%) identical to the amino acid sequence of SEQ ID NO: 32.

Embodiment 37. The isolated antibody or antigen binding fragment thereof of embodiment 36, comprising the VH comprising the amino acid sequence of SEQ ID NO: 31 and the VL comprising the amino acid sequence of SEQ ID NO: 32.

Embodiment 38. The isolated antibody or antigen binding fragment thereof of Embodiment 29, comprising the VH which is at least 80% (e.g. at least 85%, at least 90%, at least 95%, at least 99% or 100%) identical to the amino acid sequence of SEQ ID NO: 36 and the VL which is at least 80% (e.g. at least 85%, at least 90%, at least 95%, at least 99% or 100%) identical to the amino acid sequence of SEQ ID NO: 37.

Embodiment 39. The isolated antibody or antigen binding fragment thereof of embodiment 38, comprising the VH comprising the amino acid sequence of SEQ ID NO: 36 and the VL comprising the amino acid sequence of SEQ ID NO: 37.

Embodiment 40. The isolated antibody or antigen binding fragment thereof of embodiment 29, comprising the VH which is at least 80% (e.g. at least 85%, at least 90%, at least 95%, at least 99% or 100%) identical to the amino acid sequence of SEQ ID NO: 34 and the VL which is at least 80% (e.g. at least 85%, at least 90%, at least 95%, at least 99% or 100%) identical to the amino acid sequence of SEQ ID NO: 35.

Embodiment 41. The isolated antibody or antigen binding fragment thereof of embodiment 40, comprising the VH comprising the amino acid sequence of SEQ ID NO: 34 and the VL comprising the amino acid sequence of SEQ ID NO: 35.

Embodiment 42. The isolated antibody or antigen binding fragment thereof of any one of embodiments 1-41, comprising a heavy chain (HC) having an amino acid sequence at least 80% (e.g. at least 85%, at least 90%, at least 95%, or at least 99%) identical to the amino acid sequence of SEQ ID NO: 51, SEQ ID NO: 53, SEQ ID NO: 54, SEQ ID NO: 56, SEQ ID NO: 58 or SEQ ID NO: 59, and a light chain (LC) having an amino acid sequence at least 80% (e.g. at least 85%, at least 90%, at least 95%, or at least 99%) identical to the amino acid sequence of SEQ ID NO: 52, SEQ ID NO: 55, SEQ ID NO: 57 or SEQ ID NO: 60.

Embodiment 43. The isolated antibody or antigen binding fragment thereof of embodiment 42, comprising an amino acid sequence selected from the group consisting of SEQ ID NOs: 51, 52, 53, 54, 55, 56, 57, 59 and 60.

Embodiment 44. The isolated antibody of embodiment 43, comprising
a. the HC comprising the amino acid sequence at least 80% (e.g. at least 85%, at least 90%, at least 95%, at least 99% or 100%) identical to the amino acid sequence of SEQ ID NO: 51, and the LC comprising the amino acid sequence at least 80% (e.g. at least 85%, at least 90%, at least 95%, at least 99% or 100%) identical to the amino acid sequence of SEQ ID NO: 52;
b. the HC comprising the amino acid sequence at least 80% (e.g. at least 85%, at least 90%, at least 95%, at least 99% or 100%) identical to the amino acid sequence of SEQ ID NO: 53, and the LC comprising the amino acid sequence at least 80% (e.g. at least 85%, at least 90%, at least 95%, at least 99% or 100%) identical to the amino acid sequence of SEQ ID NO: 52;
c. the HC comprising the amino acid sequence at least 80% (e.g. at least 85%, at least 90%, at least 95%, at least 99% or 100%) identical to the amino acid sequence of SEQ ID NO: 54, and the LC comprising the amino acid sequence at least 80% (e.g. at least 85%, at least 90%, at least 95%, at least 99% or 100%) identical to the amino acid sequence of SEQ ID NO: 55;
d. the HC comprising the amino acid sequence at least 80% (e.g. at least 85%, at least 90%, at least 95%, at least 99% or 100%) identical to the amino acid sequence of SEQ ID NO: 56, and the LC comprising the amino acid sequence at least 80% (e.g. at least 85%, at least 90%, at least 95%, at least 99% or 100%) identical to the amino acid sequence of SEQ ID NO: 57;
e. the HC comprising the amino acid sequence at least 80% (e.g. at least 85%, at least 90%, at least 95%, at least 99% or 100%) identical to the amino acid sequence of SEQ ID NO: 58, and the LC comprising the amino acid sequence at least 80% (e.g. at least 85%, at least 90%, at least 95%, at least 99% or 100%) identical to the amino acid sequence of SEQ ID NO: 57; or
f. the HC comprising the amino acid sequence at least 80% (e.g. at least 85%, at least 90%, at least 95%, at least 99% or 100%) identical to the amino acid sequence of SEQ ID NO: 59, and the LC comprising the amino acid sequence at least 80% (e.g. at least 85%, at least 90%, at least 95%, at least 99% or 100%) identical to the amino acid sequence of SEQ ID NO: 60.

Embodiment 45. The isolated antibody of embodiment 44, comprising the HC comprising the amino acid sequence of SEQ ID NO: 51 and the LC comprising the amino acid sequence of SEQ ID NO: 52.

Embodiment 46. The isolated antibody of embodiment 44, comprising the HC comprising the amino acid sequence of SEQ ID NO: 53 and the LC comprising the amino acid sequence of SEQ ID NO: 52.

Embodiment 47. The isolated antibody of embodiment 44, comprising the HC comprising the amino acid sequence of SEQ ID NO: 54 and the LC comprising the amino acid sequence of SEQ ID NO: 55.

Embodiment 48. The isolated antibody of embodiment 44, comprising the HC comprising the amino acid sequence of SEQ ID NO: 56 and the LC comprising the amino acid sequence of SEQ ID NO: 57.

Embodiment 49. The isolated antibody of embodiment 44, comprising the HC comprising the amino acid sequence of SEQ ID NO: 58 and the LC comprising the amino acid sequence of SEQ ID NO: 57.

Embodiment 50. The isolated antibody of embodiment 44, comprising the HC comprising the amino acid sequence of SEQ ID NO: 59 and the LC comprising the amino acid sequence of SEQ ID NO: 60.

Embodiment 51. An isolated antibody or antigen binding fragment thereof, comprising
a. the HCDRs of a VH comprising the amino acid sequence of SEQ ID NO: 33, and the LCDRs of a VL comprising the amino acid sequence of SEQ ID NO: 32;
b. a VH comprising a HCDR1, a HCDR2, and a HCDR3 having an amino acid sequence of SEQ ID NOs: 7, 8, and 9, respectively, and a VL comprising a LCDR1, LCDR2, and LCDR3 having an amino acid sequence of SEQ ID NOs: 10, 11, and 12, respectively;
c. a VH having an amino acid sequence of SEQ ID NO: 33 and a VL having an amino acid sequence of SEQ ID NO: 32; and/or
d. a HC having an amino acid sequence of SEQ ID NO: 53; and a LC having an amino acid sequence of SEQ ID NO: 52.

Embodiment 52. An isolated antibody or antigen binding fragment thereof, comprising
a. the HCDRs of a VH comprising the amino acid sequence of SEQ ID NO: 34, and the LCDRs of a VL comprising the amino acid sequence of SEQ ID NO: 35;
b. a VH comprising a HCDR1, a HCDR2, and a HCDR3 having an amino acid sequence of SEQ ID NOs: 13, 14, and 15, respectively, and a VL comprising a LCDR1, LCDR2, and LCDR3 having an amino acid sequence of SEQ ID NOs: 16, 17 and 18, respectively;
c. a VH having an amino acid sequence of SEQ ID NO: 34 and a VL having an amino acid sequence of SEQ ID NO: 35; and/or d. HC having an amino acid sequence of SEQ ID NO: 54; and a LC having an amino acid sequence of SEQ ID NO: 55

Embodiment 53. An isolated antibody or antigen binding fragment thereof, comprising
a. a heavy chain variable region (VH) having the HCDRs of a VH comprising the amino acid sequence of SEQ ID NO: 38, and light chain variable region (VL) having the LCDRs of a VL comprising the amino acid sequence of SEQ ID NO: 37;
b. a VH comprising a HCDR1, a HCDR2, and a HCDR3 having an amino acid sequence of SEQ ID NOs: 19, 20, and 21, respectively, and a VL comprising a LCDR1, LCDR2, and LCDR3 having an amino acid sequence of SEQ ID NOs: 22, 23, and 24, respectively;
c. a VH having an amino acid sequence of SEQ ID NO: 38 and a VL having an amino acid sequence of SEQ ID NO: 37; and/or
d. a HC having an amino acid sequence of SEQ ID NO: 58; and a LC having an amino acid sequence of SEQ ID NO: 57.

Embodiment 54. An isolated antibody or antigen binding fragment thereof, comprising
a. a heavy chain variable region (VH) having the HCDRs of a VH comprising the amino acid sequence of SEQ ID NO: 39, and light chain variable region (VL) having the LCDRs of a VL comprising the amino acid sequence of SEQ ID NO: 40;
b. a VH comprising a HCDR1, a HCDR2, and a HCDR3 having an amino acid sequence of SEQ ID NOs: 25, 26, and 27, respectively, and a VL comprising a LCDR1, LCDR2, and LCDR3 having an amino acid sequence of SEQ ID NOs: 28, 29, and 30, respectively;
c. a VH having an amino acid sequence of SEQ ID NO: 39 and a VL having an amino acid sequence of SEQ ID NO: 40; and/or
d. a HC having an amino acid sequence of SEQ ID NO: 59 and a LC having an amino acid sequence of SEQ ID NO: 60.

Embodiment 55. An isolated antibody or antigen binding fragment thereof, comprising
a. a heavy chain variable region (VH) having the HCDRs of a VH comprising the amino acid sequence of SEQ ID NO: 31, and light chain variable region (VL) having the LCDRs of a VL comprising the amino acid sequence of SEQ ID NO: 32;
b. a VH comprising a HCDR1, a HCDR2, and a HCDR3 having an amino acid sequence of SEQ ID NOs: 7, 175, and 9, respectively, and a VL comprising a LCDR1, LCDR2, and LCDR3 having an amino acid sequence of SEQ ID NOs: 10, 11, and 12, respectively;
c. a VH having an amino acid sequence of SEQ ID NO: 31 and a VL having an amino acid sequence of SEQ ID NO: 32; and/or
d. a HC having an amino acid sequence of SEQ ID NO: 51 and a LC having an amino acid sequence of SEQ ID NO: 52.

Embodiment 56. An isolated antibody or antigen binding fragment thereof, comprising
a. a heavy chain variable region (VH) having the HCDRs of a VH comprising the amino acid sequence of SEQ ID NO: 36, and light chain variable region (VL) having the LCDRs of a VL comprising the amino acid sequence of SEQ ID NO: 37;
b. a VH comprising a HCDR1, a HCDR2, and a HCDR3 having an amino acid sequence of SEQ ID NOs: 19, 20 and 21, respectively, and a VL comprising a LCDR1, LCDR2, and LCDR3 having an amino acid sequence of SEQ ID NOs: 22, 23, and 24, respectively;
c. a VH having an amino acid sequence of SEQ ID NO: 36 and a VL having an amino acid sequence of SEQ ID NO: 37; and/or
d. a HC having an amino acid sequence of SEQ ID NO: 56 and a LC having an amino acid sequence of SEQ ID NO: 57.

Embodiment 57. The isolated antigen binding fragment of any one of embodiments 1-56, wherein the antigen binding fragment is a scFv, a (scFv) 2, a Fv, a Fab, a F(ab′) 2, a Fd, a dAb, a VHH or single chain antibody.

Embodiment 58. The isolated antibody or antigen binding fragment thereof of any one of embodiment 1-57, wherein the antibody or antigen binding fragment thereof is of an IgG1, an IgG2, an IgG3 or an IgG4 isotype.

Embodiment 59. The isolated antibody or antigen binding fragment thereof of embodiment 58, wherein the antibody or antigen binding fragment thereof is an IgG1 isotype.

Embodiment 60. The isolated antibody or antigen binding fragment thereof of embodiment 59, comprising an Ig constant region or a fragment of the Ig constant region.

Embodiment 61. The isolated antibody or antigen binding fragment thereof of embodiment 60, wherein the Ig constant region or the fragment of the Ig constant region comprises at least one mutation that results in reduced binding of the antibody or antigen binding fragment thereof to a Fcγ receptor (FcγR).

Embodiment 62. The isolated antibody or antigen binding fragment thereof of embodiment 61, wherein the at least one mutation that results in reduced binding of the antibody or antigen binding fragment thereof to a FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

Embodiment 63. The isolated antibody or antigen binding fragment thereof of embodiment 62, wherein the Ig constant region or the fragment of the constant region comprises the mutations of L234A_L235A_D265S.

Embodiment 64. The isolated antibody or antigen binding fragment thereof of embodiment 62, wherein the FcγR is FcγRI, FcγRIIA, FcγRIIB or FcγRIII, or any combination thereof.

Embodiment 65. The isolated antibody or antigen binding fragment thereof of embodiment 64, wherein the Ig constant region or the fragment of the Ig constant region further comprises at least one mutation that modulates a half-life of the antibody or antigen binding fragment thereof.

Embodiment 66. The isolated antibody or antigen binding fragment thereof of embodiment 65, wherein the at least one mutation that modulates the half-life of the antibody or antigen binding fragment thereof is selected from the group consisting of H435A, P257I/N434H, D376V/N434H, M252Y/S254T/T256E/H433K/N434F, T308P/N434A and H435R, wherein residue numbering is according to the EU index.

Embodiment 67. An immunoconjugate comprising the isolated antibody or antigen binding fragment thereof of any one of embodiment 1-66 conjugated to a therapeutic agent or an imaging agent.

Embodiment 68. A pharmaceutical composition comprising the isolated antibody or antigen binding fragment thereof of any one of embodiment 1-66 or the immunoconjugate of embodiment 67, and a pharmaceutically acceptable carrier.

Embodiment 69. An isolated polynucleotide encoding the isolated antibody or antigen binding fragment thereof of any one of embodiments 1-66.

Embodiment 70. The isolated polynucleotide of embodiment 69, comprising a polynucleotide sequence at least 80%, such as at least 85%, at least 90%, at least 95%, at least 99% or 100%, identical to the polynucleotide sequence of SEQ ID NOs: 41, 42, 43, 44, 45, 46, 47, 48, 49, 50, 61, 62, 63, 64, 65, 66, 67, 68, 69, or 70.

Embodiment 71. A vector comprising the polynucleotide of embodiment 69 or 70.

Embodiment 72. A host cell expressing the isolated antibody or antigen binding fragment thereof of any one of embodiments 1-70, such as a host cell comprising the vector of embodiment 71.

Embodiment 73. A method of preventing the binding of VEGFA to a VEGFR1 in a subject in need thereof, comprising administering to the subject an effective amount of the isolated antibody or antigen fragment thereof of any one of embodiments 1-66, the immunoconjugate of embodiment 67, the pharmaceutical composition of embodiment 68, the isolated polynucleotide of embodiment 69 or 70, the vector of embodiment 71 or the host cell of embodiment 72 to thereby prevent the binding of VEGFA to the VEGFR1.

Embodiment 74. The method of embodiment 73, wherein the subject is in need of a treatment of a Chronic Kidney Disease (CKD), in need of decreasing proteinuria, has advanced stage 4 or 5 chronic kidney disease, or has CKD with proteinuria, albuminuria, or diabetes mellitus.

Embodiment 75. A method of treating a Chronic Kidney Disease (CKD) in a subject in need thereof, comprising administering a therapeutically effective amount of the isolated antibody or antigen fragment thereof any one of embodiments 1-66, the immunoconjugate of embodiment 67, the pharmaceutical composition of embodiment 68, the isolated polynucleotide of embodiment 69 or 70, the vector of embodiment 71 or the host cell of embodiment 72 to the subject for a time sufficient to treat the CKD.

Embodiment 76. The method of embodiment 75, wherein the subject has advanced stage 4 or 5 chronic kidney disease.

Embodiment 77. The method of embodiment 75, wherein the subject has stage 3 chronic kidney disease.

Embodiment 78. The method of embodiment 75, wherein the subject has CKD with proteinuria, albuminuria, or diabetes mellitus.

Embodiment 79. A method of decreasing proteinuria in a subject in need thereof, comprising administering a therapeutically effective amount of the isolated antibody or antigen fragment thereof of any one of embodiments 1-66, the immunoconjugate of embodiment 67, the pharmaceutical composition of embodiment 68, the isolated polynucleotide of embodiment 69 or 70, the vector of embodiment 71 or the host cell of embodiment 72, to the subject for a time sufficient to decrease proteinuria in the subject.

Embodiment 80. A method of decreasing albuminuria in a subject in need thereof, comprising administering a therapeutically effective amount of the isolated antibody or antigen fragment thereof of any one of embodiments 1-66, the immunoconjugate of embodiment 67, the pharmaceutical composition of embodiment 68, the isolated polynucleotide of embodiment 69 or 70, the vector of embodiment 71 or the host cell of embodiment 72, to the subject for a time sufficient to decrease proteinuria in the subject.

Embodiment 81. A kit comprising the isolated antibody or antigen binding fragment thereof of any one of embodiments 1-66, the immunoconjugate of embodiment 67, the pharmaceutical composition of embodiment 68, the isolated polynucleotide of embodiment 69 or 70, the vector of embodiment 71 or the host cell of embodiment 72.

### EXAMPLES

### Example 1. Antigen Generation

Vascular endothelial growth factor receptor 1 (VEGFR1) is a member of the type V subfamily of receptor tyrosine kinases (RTK) comprised of seven extracellular immunoglobulin (Ig)-like domains, a single transmembrane domain, and a cytoplasmic kinase domain. Structural investigations previously demonstrated that ligand binding to VEGFR1 is mediated by Ig domains 2 and 3 (D2 and D3) with the two domains contributing ˜800 and 700 Å² to the binding interface, respectively (Markovic-Mueller, S. et al, Structure, 25 (2), 341-352, 2017). This was corroborated by binding analyses in which the affinity of recombinant human VEGFR1 (D1-D3) and human VEGFR1 (D2-D3) for human VEGF165 (R&D Systems) was determined by SPR to be <45 and <30 pM, respectively. Domain 2 of hVEGFR1 alone exhibited a significantly lower affinity of 22 nM (˜1,000-fold reduction in affinity), which was consistent with previously published biochemical data demonstrating that D2 of VEGFR1 alone (Flt-1 (2)-IgG) exhibits substantially reduced affinity for VEGF165 (Davis-Smyth, T. et al, EMBO Journal, 15 (18), 4919-4927, 1996).

Therefore, to identify antibodies capable of blocking ligand binding to VEGFR1, truncated versions of the VEGFR1 extracellular domain (ECD) comprising only D2-D3 or D1-D3 were used as the primary immunogens during antibody discovery. An Fc chimera of the second and third immunoglobulin (Ig)-like domains of human VEGFR1 (hVEGFR1 D2-D3-Fc-6×His) was produced by fusing amino acid residues 130-331 (UniProt Accession #P17948) to residues 100-330 of human immunoglobulin heavy constant gamma 1 (UniProt Accession #P01857) through the HRV3C protease recognition sequence ‘LEVLFQGP’ (SEQ ID NO: 174). A 6×His-tag was added to the C-terminus of the construct for flexible purification options (construct VGFW8; SEQ ID NO: 1). Additionally, an Fc chimera of the first through the third immunoglobulin (Ig)-like domains of murine VEGFR1 (mVEGFR1 D1-D3-Fc-6×His) was produced by connecting residues 23-332 (UniProt Accession #P35969) to residues 100-330 of human immunoglobulin heavy constant gamma 1 (UniProt Accession #P01857) through the HRV3C protease recognition sequence ‘LEVLFQGP’ (SEQ ID NO: 174).

A similar construct (mFlt (1-3-IgG) was previously described as a potent inhibitor of VEGF and/or PlGF activities (Ferrara, N. et al, Nature Medicine, 4 (3), 336-340 1998). A 6×His-tag was also added to the C-terminus of the construct for flexibility in purification strategy (construct VGFW9; SEQ ID NO: 2). Additional constructs were designed in which portions or the entirety of the VEGFR1 extracellular domain (ECD) from human, mouse, or cynomolgus monkey (Macaca fascicularis) were appended at the C-terminus with Avi-tag and 6×His-tag sequences. These included: Ig-like domain 2 of human VEGFR1 (residues 130-225 of Uniprot Accession #P17948; construct VGFW2; SEQ ID NO: 3), Ig-like domains 2 and 3 of human VEGFR1 (residues 130-331 of Uniprot Accession #P17948; construct VGFW3; SEQ ID NO: 4), Ig-like domains 1 thru 3 of murine VEGFR1 (residues 23-332 of Uniprot Accession #P35969; construct VGFW4; SEQ ID NO: 5), and the entire extracellular domain of monkey VEGFR1 (residues 27-758 of Accession #XP_005585612.1; construct VGFW5; SEQ ID NO: 6).

The VEGFR1 ECD expression constructs were transiently transfected into HEK 293-6E cells, using polyethyleneimine. Cells were incubated for six days at 37° C. with 5% CO₂ in a batch-fed Wave bag (Culti bag) 20L [Sartorius, Flexsafe@ RM 20L optical Cat #DFO020L] prior to harvesting when the viability of the culture had dropped to <80%. The cells were removed by centrifugation and the soluble VEGFR1 proteins with His-tags were purified from the media using immobilized metal affinity chromatography (IMAC) using Ni Sepharose 6 Fast Flow resin (GE Healthcare) followed by preparative size exclusion chromatography (SEC) using a Superdex 200 column (GE Healthcare) equilibrated in Dulbecco's Phosphate Saline buffer pH 7.2 (1×DPBS). Alternatively, for VGFW8 and VGFW9, the recombinant protein was captured from the spent supernatant by application to and elution from MabSelect SuRe protein A chromatography resin (GE Healthcare). The amino acid sequences of the generated antigens are shown in Table 2.

TABLE 2

| | | SEQ | |
|---|---|---|---|
| Construct | | ID | |
| ID | Description | NO: | Amino Acid Sequence |
| VGFW8 | hVEGFR1_D2-3_ | 1 | DTGRPFVEMYSEIPEIIHMTEGRELVIPCR |
| | HRV3C_FC (Human | | VTSPNITVTLKKFPLDTLIPDGKRIIWDSR |
| | IgG1-6xHis | | KGFIISNATYKEIGLLTCEATVNGHLYKT |
| | | | NYLTHRQTNTIIDVQISTPRPVKLLRGHTL |
| | | | VLNCTATTPLNTRVQMTWSYPDEKNKR |
| | | | ASVRRRIDQSNSHANIFYSVLTIDKMQNK |
| | | | DKGLYTCRVRSGPSFKSVNTSVHIYDKLE |
| | | | VLFQGPPKSCDKTHTCPPCPAPELLGGPS |
| | | | VFLFPPKPKDTLMISRTPEVTCVVVDVSH |
| | | | EDPEVKFNWYVDGVEVHNAKTKPREEQ |
| | | | YNSTYRVVSVLTVLHQDWLNGKEYKCK |
| | | | VSNKALPAPIEKTISKAKGQPREPQVYTL |
| | | | PPSRDELTKNQVSLTCLVKGFYPSDIAVE |
| | | | WESNGQPENNYKTTPPVLDSDGSFFLYS |
| | | | KLTVDKSRWQQGNVFSCSVMHEALHNH |
| | | | YTQKSLSLSPGKHHHHHH |
| VGFW9 | mVEGFR1_D1-3_ | 2 | YGSGSKLKVPELSLKGTQHVMQAGQTLF |
| | HRV3C_FC(Human | | LKCRGEAAHSWSLPTTVSQEDKRLSITPP |
| | IgG1)-6xHis | | SACGRDNRQFCSTLTLDTAQANHTGLYT |
| | | | CRYLPTSTSKKKKAESSIYIFVSDAGSPFIE |
| | | | MHTDIPKLVHMTEGRQLIIPCRVTSPNVT |
| | | | VTLKKFPFDTLTPDGQRITWDSRRGFIIAN |
| | | | ATYKEIGLLNCEATVNGHLYQTNYLTHR |
| | | | QTNTILDVQIRPPSPVRLLHGQTLVLNCT |
| | | | ATTELNTRVQMSWNYPGKATKRASIRQR |
| | | | IDRSHSHNNVFHSVLKINNVESRDKGLYT |
| | | | CRVKSGSSFQSFNTSVHVYEKLEVLFQGP |
| | | | PKSCDKTHTCPPCPAPELLGGPSVFLFPPK |
| | | | PKDTLMISRTPEVTCVVVDVSHEDPEVKF |
| | | | NWYVDGVEVHNAKTKPREEQYNSTYRV |
| | | | VSVLTVLHQDWLNGKEYKCKVSNKALP |
| | | | APIEKTISKAKGQPREPQVYTLPPSRDELT |
| | | | KNQVSLTCLVKGFYPSDIAVEWESNGQP |
| | | | ENNYKTTPPVLDSDGSFFLYSKLTVDKSR |
| | | | WQQGNVFSCSVMHEALHNHYTQKSLSL |
| | | | SPGKHHHHHH |
| VGFW2 | hVEGFR1_D2-Avi-His | 3 | DTGRPFVEMYSEIPEIIHMTEGRELVIPCR |
| | | | VTSPNITVTLKKFPLDTLIPDGKRIIWDSR |
| | | | KGFIISNATYKEIGLLTCEATVNGHLYKT |
| | | | NYLTHRQGLNDIFEAQKIEWHEHHHHHH |
| VGFW3 | hVEGFR1_D2-3 Avi His | 4 | DTGRPFVEMYSEIPEIIHMTEGRELVIPCR |
| | | | VTSPNITVTLKKFPLDTLIPDGKRIIWDSR |
| | | | KGFIISNATYKEIGLLTCEATVNGHLYKT |
| | | | NYLTHRQTNTIIDVQISTPRPVKLLRGHTL |
| | | | VLNCTATTPLNTRVQMTWSYPDEKNKR |
| | | | ASVRRRIDQSNSHANIFYSVLTIDKMQNK |
| | | | DKGLYTCRVRSGPSFKSVNTSVHIYDKGL |
| | | | NDIFEAQKIEWHEHHHHHH |
| VGFW4 | mVEGFR1_D1-3_Avi- | 5 | YGSGSKLKVPELSLKGTQHVMQAGQTLF |
| | 6xHis | | LKCRGEAAHSWSLPTTVSQEDKRLSITPP |
| | | | SACGRDNRQFCSTLTLDTAQANHTGLYT |
| | | | CRYLPTSTSKKKKAESSIYIFVSDAGSPFIE |
| | | | MHTDIPKLVHMTEGRQLIIPCRVTSPNVT |
| | | | VTLKKFPFDTLTPDGQRITWDSRRGFIIAN |
| | | | ATYKEIGLLNCEATVNGHLYQTNYLTHR |
| | | | QTNTILDVQIRPPSPVRLLHGQTLVLNCT |
| | | | ATTELNTRVQMSWNYPGKATKRASIRQR |
| | | | IDRSHSHNNVFHSVLKINNVESRDKGLYT |
| | | | CRVKSGSSFQSFNTSVHVYEKGLNDIFEA |
| | | | QKIEWHEHHHHHH |
| VGFW5 | cynoVEGFR1_ECD_Avi- | 6 | SKLKGPELSLKGTQHVTQAGQTLHLQCR |
| | 6xHis | | GEAAHQWSLPETVSKESKRLSITKSACGR |
| | | | NGKQFCSTLTLNAAQANHTGFYSCKYLA |
| | | | VPTSKKKETESAIYIFISDTGRPFVEMYSEI |
| | | | PEIIHMTEGRELIIPCRVTSPNITVTLKKFP |
| | | | LDTLIPDGKRVIWDSRKGFIISNATYKEIG |
| | | | LLTCEATVNGHLYKTNYLTHRQTNTIIDV |
| | | | QISTPRPVKLLRGHTLILNCTATTPLNTRV |
| | | | QMTWSYPDEKNKRASVRRRIDQSNSHAN |
| | | | IFYSVLTIDKVQNKDKGLYTCRVRSGPSF |
| | | | KSVNTSVHIYDKAFITVKHRKQQVLETV |
| | | | AGKRSYRLSMKVKAFPSPEVVWLKDGLP |
| | | | ATEKSARYLTRGYSLIIKDVTEEDAGNYT |
| | | | ILLSIKQSNVFKNLTATLIVNVKPQIYEKA |
| | | | VSSFPDPALYPLGSRQILTCTAYGIPRPTIK |
| | | | WCWRPCNHNHSEARYDFCSNNEESFILD |
| | | | PDSNIGNRIESITQRMAIIEGKNKTASTLV |
| | | | VADSRISGIYSCIASNKVGTVGRNISFYIT |
| | | | DVPNGFHVNLEKMPTEGEDLKLSCTVNK |
| | | | FLYRDVTWILLRTVNNRTMHYSISKQKM |
| | | | AITKEHSITLNLTIMNVSLEDSGTYACRA |
| | | | RNVYTGEEILQKKEVTIRDQEAPYLLRNL |
| | | | SDHTVAISSSTTLDCHANGAPEPQITWFK |
| | | | NNHKIQQEPGIILGPGSSTLFIERVTEEDEG |
| | | | VYHCKATNQKGSVESSAYLTVQGTSDKS |
| | | | NLEGLNDIFEAQKIEWHEHHHHHH |

### Example 2. VEGFR1 Antibody Generation and Preliminary Characterization in scFv-Fc Format

### Immunization Strategy

There was a strong preference to identify ligand-blocking, anti-VEGFR1 antibodies that exhibited broad cross-reactivity across various species (in addition to human) including cynomolgus monkey as well as rodents to enable both non-clinical safety and pharmacology/efficacy studies in well-established rodent models of diabetic kidney disease (DKD). The ability to generate species cross-reactive antibodies at the start of the discovery campaign eliminated the need to develop surrogate antibodies which may not have mimicked the behavior of the antibody of interest given the complexity of the biology.

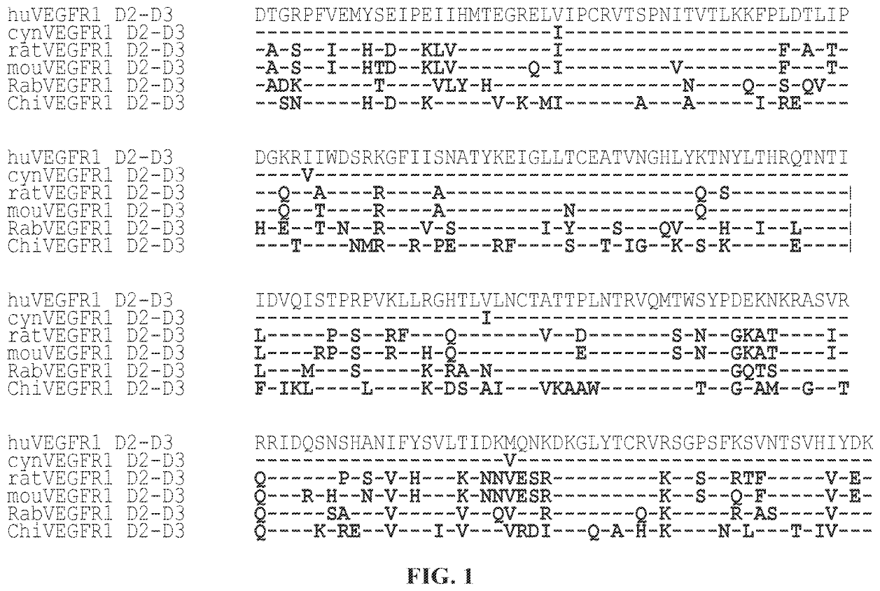

Obtaining antibodies with broad species cross-reactivity (including rodent) can represent a stringent design requirement. Indeed, previously reported anti-VEGFR1 antibodies were either strictly mouse specific or strictly human specific and showed poor to no cross-species reactivity. VGFB80 used herein as a reference does not exhibit binding to rodent derived VEGFR1 though it is a potent human VEGFR1 binder. It is well-established that the generation of broadly cross-reactive antibodies is often hampered by immunological tolerance to epitopes which are conserved across mammalian species typically used for antibody discovery (e.g., mice, rats, and rabbits). The conservation of the ligand-binding domain of VEGFR1 across typical antibody discovery species (i.e. mouse and rat) is evidenced by the amino acid sequence identities which are shown in FIG. 1 and Table 3. Sequence identities of ˜80 and 67% are observed between human and mouse/rat for VEGFR1 domains 2 and 3, respectively.

TABLE 3
Summary of VEGFR1 sequence identity of Domain 2 or Domains
2 and 3 between human and cyno, mouse, rat, rabbit,
or chicken. Percent identity was calculated by alignment
and analysis using Geneious Prime ® 2021.1.1.

| | | VEGFR1 D2 | | VEGFR1 D2-D3 | |
|---|---|---|---|---|---|
| | | Seq | % ID to | Seq | % ID to |
| Species | Accession # | Range | human | Range | human |
| Human | P17948 | 133-228 | 100.0 | 133-334 | 100.0 |
| Cyno | XP_005585612.1 | 130-225 | 97.9 | 130-331 | 98.0 |
| Rat | P53767 | 130-225 | 81.3 | 130-331 | 74.3 |
| Mouse | P35969 | 131-226 | 79.2 | 131-332 | 73.3 |
| Rabbit | XP_008273438.1 | 132-227 | 70.8 | 132-333 | 73.8 |
| Chicken | Q8QHL3 | 126-221 | 67.7 | 126-327 | 64.4 |

Alternatively, the immunization of more phylogenetically distant species such as chickens can sometimes overcome immunological tolerance to “pan-mammalian” epitopes (Ching, K. H. et al, MAbs, 10 (1), 71-80, 2018). Transgenic Omnichicken, which are more phylogenetically distant to humans compared to mice and rats, have demonstrated robust immune responses to conserved human proteins. However, the use of Omnichicken alone does not guarantee high affinity species cross-reactive antibodies.

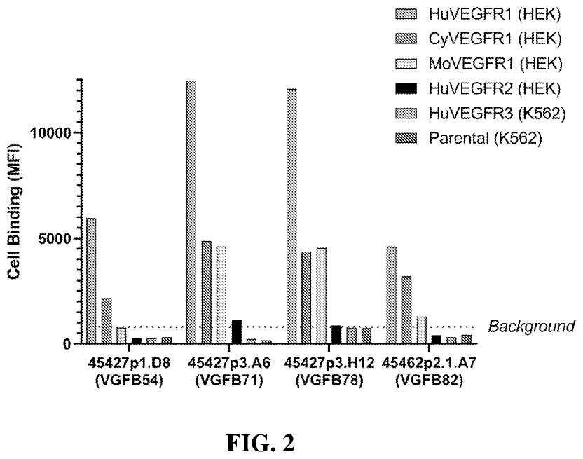

The high affinity and species cross-reactive anti-VEGFR1 antibodies described herein were generated using a unique antibody generation strategy involving (1) the selection and design of distinctive antigens (ligand-binding subdomain of VEGFR1), (2) the choice of phylogenetically distant species for immunization (chicken), (3) a unique immunization procedure and a (4) meticulous serum titer screening process, that identify anti-VEGFR1 antibodies with broad species reactivity. Transgenic chickens (n=12) possessing humanized immunoglobulin genes (OmniChicken; Ligand Pharmaceuticals; Emeryville, CA and Schusser, B. et al, Proc. Natl. Acad. Sci. USA 110, 20170-20175, 2013) were immunized with either hVEGFR1 D2-D3-Fc-6×His (VGFW8) or with alternating boosts of VGFW8 and mVEGFR1 D1-D3-Fc-6×His (VGFW9). Serum titers of the birds were monitored by ELISA using VGFW2 (hVEGFR1_D2-Avi-His), VGFW3 (hVEGFR1_D2-3 Avi His), VGFW4 (mVEGFR1_D1-3_Avi-6×His), and VGFW5 (cynoVEGFR1_ECD_Avi-6×His) purified recombinant proteins. Spleens were harvested from the birds with the best titers against both human and mouse VEGFR1 recombinant protein. Lymphocytes were isolated from the spleens and interrogated for positive binding to both beads coated with VGFW4 as well as VGFW3 and/or VGFW2. Single-cell reverse transcriptase PCR was performed on lymphocytes to recover v-gene sequences. VH and VL sequences were assembled as scFv-Fc (human IgG1) molecules in a mammalian expression vector. The mammalian expression constructs were used to produce the antibodies at small-scale (96-well format) by transient transfection. The supernatants from small-scale expression were screened for the presence of recombinant antibodies with specificity for VEGFR1 by ELISA using purified antigen. Re-confirmed positive clones were expanded to 6-well plates (2 mL) and serial dilutions of the supernatants were used to repeat ELISA and FACS analysis to confirm binding to human, cyno, and mouse VEGFR1-both as soluble purified recombinant protein of the extracellular domains and as full-length cell surface-bound receptors on HEK cells engineered to over-express either human, cyno, or mouse VEGFR1. Additionally, the expanded clones were counter-screened on HEK and K562 cells engineered to over-express hVEGFR2 and hVEGFR3, respectively (FIG. 2).

The subset of clones which displayed specific binding to human, cyno, and mouse VEGFR1 by ELISA and FACS were sequenced and reformatted as human monoclonal IgG1 (L234A/L235A/D265S) molecules (2H: 2L) to abrogate any Fc receptor effector function while preserving FcRn affinity. Out of ˜800 lymphocytes that were initially interrogated, 43 unique molecules resulted which met the criteria described above (5.6%). The remaining clones generally either lacked cross-reactivity to murine VEGFR1 or did not show specific binding to VEGFR1 (i.e., also bound human VEGFR2 or human VEGFR3 on cells). In contrast, a more conventional rodent immunization campaign was conducted in parallel (n=42 animals immunized) and of ˜2,200 MSD primary hits screened on cells, there were only 4 clones that demonstrated Hu/Cy/Mo VEGFR1 broad species cross-reactivity (0.18% hit rate)—and these molecules ultimately proved to be less effective at blocking ligand binding or possessed undesired sequence liabilities.

The 43 unique Omnichicken-derived clones reformatted as human monoclonal IgG1 (L234A/L235A/D265S) molecules were expressed and purified at small scale (2 mL) and subjected to extensive additional characterization including (1) affinity determination against human, cynomolgus monkey, and mouse VEGFR1 by SPR (2) ligand blocking potency (both biochemical and cell-based), (3) thermal stability, (4) surface hydrophobicity, (5) non-specific binding, (6) lack of modification following stress/forced degradation studies. 28 of the 43 molecules (˜65%) were identified as potent ligand blockers representing 10-11 sequence families. Eight anti-VEGFR1 mAbs were evaluated in a single-dose PK-TE-PD study in db/db mice. VGFB54, VGFB71, VGFB78 and VGFB82 were evaluated in a cynomolgus monkeys single dose PK-TE study and subjected to an extensive panel of intrinsic property and structural characterizations.

### Design of VGFB883 and VGFB400

VGFB54 was observed to contain an ‘NG’ deamidation sequence liability in heavy chain CDR2, and was observed by peptide mapping to undergo significant deamidation (14.7% modification) when maintained at pH 8.5 (40 C) for one week. To address this risk, a small library of variants was generated in which either N54 or G55 were substituted with every possible alternative amino acid (except Cys or Trp). The library of variants was then evaluated for retained binding to target both in the biochemical ligand blocking assay and in the cell binding assay. Nearly all variants exhibited retained or slightly improved binding to VEGFR1. The N54Q variant of VGFB54 (VGFB883) was selected for further development as it is one of the most chemically conservative amino acid substitutions, resolves the post-translational modification risk, and exhibited retention of binding to target (Table 4).

TABLE 4
Ligand blocking and cell binding of VGFB883

| Antibody | Ligand blocking, IC50 (M) | Cell binding, IC50 (M) |
|---|---|---|
| VGFB54 | 8.94095E⁻¹¹ | 1.97903E⁻¹⁰ |
| VGFB883 | 4.40442E⁻¹¹ | 6.54912E⁻¹¹ |

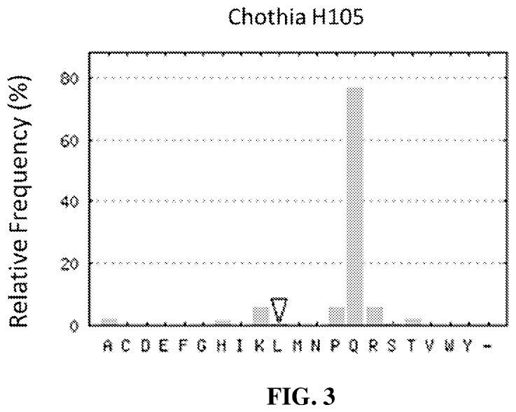

Antibody sequences obtained from transgenic animals may contain somatic hypermutations in the framework and CDR regions. Somatic hypermutations may result in unusual or low frequency residues in human framework regions and impact the stability and immunogenicity of biotherapeutics. The parent anti-VEGFR1 antibody VGFB78 heavy chain variable region contained a somatic hypermutation at position 110 (105 Chothia delineation) within the framework region. A Leucine residue at position 110 (105 Chothia) is typically a low frequency residue (frequency below 1%) compared to the most frequently found Glutamine (Q) residue (frequency 76%) typically found at that position (FIG. 3). A L11Q mutation was therefore engineered in the heavy chain of VGFB78, leading to VGFB400, which represents a reversion to the germline identity at this position. This substitution was chosen to reflect the most common identity at this position.

### ExpiCHO Small Scale Transfection and Purification

Antibodies identified from the immunization campaign were cloned and expressed as IgG1-AAS (L234A/L235A/D265S) at 2 ml scale and purified. ExpiCHO™ cells (ThermoFisher Scientific) were cultured in ExpiCHO™ Expression Medium at 37° C., 7% CO2. Cells were sub-cultured when density reached the log phase growth at 4-6×10⁶ viable cells/mL with a 98-99% viability. On the day of transfection, the viable cell density and percent viability was determined. Cells were transfected at a density of 6×10⁶ viable cells/mL following manufacturer's Transfection protocol (ThermoFisher ExpiCHO Expression System Protocols for 24 and 96 deep well blocks and mini bioreactor tubes). Culture supernatants were harvested on Day 7 post-transfection by centrifugation at 850×g for 15 minutes prior to purification. Antibodies were purified from the clarified supernatants using MabSelect Sure resin (GE Healthcare) and dialyzed into PBS. Protein concentrations were determined by measurement of absorbance a t 280 nm on the filtrate using a DropSense Instrument (Trinean NV/SA).

### Sequences of Anti-VEGFR1 Antibodies

Table 5 shows the Kabat HCDR1, HCDR2 and HCDR3 of selected anti-VEGFR1 selected antibodies. Table 6 shows the Kabat LCDR1, LCDR2 and LCDR3 of the selected anti-VEGFR1 antibodies. Table 7 shows the Chothia HCDR1, HCDR2 and HCDR3 of selected anti-VEGFR1 antibodies. Table 8 shows the Chothia LCDR1, LCDR2 and LCDR3 of selected anti-VEGFR1 antibodies. Table 9 shows the ABM HCDR1, HCDR2 and HCDR3 of selected anti-VEGFR1 antibodies. Table 10 shows the ABM LCDR1, LCDR2 and LCDR3 of selected anti-VEGFR1 antibodies. Table 11 shows the IMTG HCDR1, HCDR2 and HCDR3 of selected anti-VEGFR1 antibodies. Table 12 shows the IMTG LCDR1, LCDR2 and LCDR3 of selected anti-VEGFR1 antibodies. Table 13 shows the VH and VL amino acid sequences of selected anti-VEGFR1 antibodies. Table 14 shows the VH nucleic acid sequences of selected anti-VEGFR1 antibodies. Table 15 shows the VL nucleic acid sequences of selected anti-VEGFR1 antibodies. Table 16 shows the HC amino acid sequences of selected anti-VEGFR1 antibodies. Table 17 shows the LC amino acid sequences of selected anti-VEGFR1 antibodies. Table 18 shows the HC nucleotide sequences of selected anti-VEGFR1 antibodies. Table 19 shows the LC nucleotide sequences of selected anti-VEGFR1 antibodies. Table 20 summarizes the SEQ ID NO: assigned to the selected anti-VEGFR1 antibodies.

TABLE 5
HCDRs of selected anti-VEGFR1 antibodies using
Kabat delineation

| | | HCDR1 | | HCDR2 | | HCDR3 |
|---|---|---|---|---|---|---|
| | HCDR1 | SEQ ID | | SEQ ID | | SEQ ID |
| mAb | sequence | NO: | HCDR2 sequence | NO: | HCDR3 sequence | NO: |
| VGFB54 | DYGMN | 7 | SINRNGDISTYHVDSVKG | 175 | NHDYHGMDV | 9 |
| VGFB883 | DYGMN | 7 | SINRQGDISTYHVDSVKG | 8 | NHDYHGMDV | 9 |
| VGFB71 | DYNMN | 13 | IITNDGSSTAYSDSVKG | 14 | VYYGMDD | 15 |
| VGFB78 | NYDMT | 19 | GIGTSGTDMYYADSVKG | 20 | DYYYYGMDV | 21 |
| VGFB400 | NYDMT | 19 | GIGTSGTDMYYADSVKG | 20 | DYYYYGMDV | 21 |
| VGFB82 | TYAMN | 25 | TINSEGTITSHAPAVKG | 26 | TTGTTHGMDV | 27 |

TABLE 6
LCDRs of selected anti-VEGFR1 antibodies using
Kabat delineation

| | | LCDR1 | | LCDR2 | | LCDR3 |
|---|---|---|---|---|---|---|
| | | SEQ | LCDR2 amino | SEQ ID | | SEQ ID |
| mAb | LCDR1 amino acid | ID NO: | acid | NO: | LCDR3 sequence | NO: |
| VGFB54 | SGSRSNIGNNFVN | 10 | LNSQRPS | 11 | ASWDDSLNGWV | 12 |
| VGFB883 | SGSRSNIGNNFVN | 10 | LNSQRPS | 11 | ASWDDSLNGWV | 12 |
| VGFB71 | SGGSSNIGSNYVS | 16 | FNFQRPS | 17 | AAWDDRVNVWV | 18 |
| VGFB78 | SGSTSNIGNNYVS | 22 | YDQRPS | 23 | AAWDDSLNGWV | 24 |
| VGFB400 | SGSTSNIGNNYVS | 22 | YDORPS | 23 | AAWDDSLNGWV | 24 |
| VGFB82 | SGSSSNIQNNYVY | 28 | FNSQRPS | 29 | GTWDDSLNGWV | 30 |

TABLE 7
HCDRs of selected anti-VEGFR1 antibodies using
Chothia delineation

| | | HCDR1 | | HCDR2 | | HCDR3 |
|---|---|---|---|---|---|---|
| | HCDR1 | SEQ ID | | SEQ ID | | SEQ ID |
| mAb | sequence | NO: | HCDR2 sequence | NO: | HCDR3 sequence | NO: |
| VGFB54 | GFNFEDY | 71 | NRNGDIS | 176,. | NHDYHGMDV | 73 |
| VGFB883 | GFNFEDY | 71 | NRQGDIS | 72 | NHDYHGMDV | 73 |
| VGFB71 | GFIFRDY | 77 | TNDGSS | 78 | VYYGMDD | 79 |
| VGFB78 | SFTFYNY | 83 | GTSGTD | 84 | DYYYYGMDV | 85 |
| VGFB400 | SFTFYNY | 83 | GTSGTD | 84 | DYYYYGMDV | 85 |
| VGFB82 | GFDFSTY | 89 | NSEGTI | 90 | TTGTTHGMDV | 91 |

TABLE 8
LCDRs of selected anti-VEGFR1 antibodies using Chothia delineation

| | | LCDR1 | | LCDR2 | | LCDR3 |
|---|---|---|---|---|---|---|
| | | SEQ | LCDR2 amino | SEQ ID | | SEQ ID |
| mAb | LCDR1 amino acid | ID NO: | acid | NO: | LCDR3 sequence | NO: |
| VGFB54 | SGSRSNIGNNFVN | 74 | LNSQRPS | 75 | ASWDDSLNGWV | 76 |
| VGFB883 | SGSRSNIGNNFVN | 74 | LNSQRPS | 75 | ASWDDSLNGWV | 76 |
| VGFB71 | SGGSSNIGSNYVS | 80 | FNFQRPS | 81 | AAWDDRVNVWV | 82 |
| VGFB78 | SGSTSNIGNNYVS | 86 | YDQRPS | 87 | AAWDDSLNGWV | 88 |
| VGFB400 | SGSTSNIGNNYVS | 86 | YDQRPS | 87 | AAWDDSLNGWV | 88 |
| VGFB82 | SGSSSNIQNNYVY | 92 | FNSQRPS | 93 | GTWDDSLNGWV | 94 |

TABLE 9
HCDRs of selected anti-VEGFR1 antibodies using ABM delineation

| | | HCDR1 | | HCDR2 | | HCDR3 |
|---|---|---|---|---|---|---|
| | | SEQ ID | | SEQ ID | | SEQ ID |
| mAb | HCDR1 sequence | NO: | HCDR2 sequence | NO: | HCDR3 sequence | NO: |
| VGFB54 | GFNFEDYGMN | 95 | SINRNGDISTY | 177 | NHDYHGMDV | 97 |
| VGFB883 | GFNFEDYGMN | 95 | SINRQGDISTY | 96 | NHDYHGMDV | 97 |
| VGFB71 | GFIFRDYNMN | 101 | IITNDGSSTA | 102 | VYYGMDD | 103 |
| VGFB78 | SFTFYNYDMT | 107 | GIGTSGTDMY | 108 | DYYYYGMDV | 109 |
| VGFB400 | SFTFYNYDMT | 107 | GIGTSGTDMY | 108 | DYYYYGMDV | 109 |
| VGFB82 | GFDFSTYAMN | 113 | TINSEGTITS | 114 | TTGTTHGMDV | 115 |

TABLE 10
LCDRs of selected anti-VEGFR1 antibodies using ABM delineation

| | | LCDR1 | | LCDR2 | | LCDR3 |
|---|---|---|---|---|---|---|
| | | SEQ | LCDR2 amino | SEQ ID | | SEQ |
| mAb | LCDR1 amino acid | ID NO: | acid | NO: | LCDR3 sequence | ID NO: |
| VGFB54 | SGSRSNIGNNFVN | 98 | LNSQRPS | 99 | ASWDDSLNGWV | 100 |
| VGFB883 | SGSRSNIGNNFVN | 98 | LNSQRPS | 99 | ASWDDSLNGWV | 100 |
| VGFB71 | SGGSSNIGSNYVS | 104 | FNFQRPS | 105 | AAWDDRVNVWV | 106 |
| VGFB78 | SGSTSNIGNNYVS | 110 | YDQRPS | 111 | AAWDDSLNGWV | 112 |
| VGFB400 | SGSTSNIGNNYVS | 110 | YDQRPS | 111 | AAWDDSLNGWV | 112 |
| VGFB82 | SGSSSNIQNNYVY | 116 | FNSQRPS | 117 | GTWDDSLNGWV | 118 |

TABLE 11
HCDRs of selected anti-VEGFR1 antibodies using
IMTG delineation

| | | HCDR1 | | HCDR2 | | HCDR3 |
|---|---|---|---|---|---|---|
| | HCDR1 | SEQ ID | HCDR2 | SEQ ID | | SEQ ID |
| mAb | sequence | NO: | sequence | NO: | HCDR3 sequence | NO: |
| VGFB54 | GFNFEDYG | 119 | INRNGDIST | 178 | TKNHDYHGMDV | 121 |
| VGFB883 | GFNFEDYG | 119 | INRQGDIST | 120 | TKNHDYHGMDV | 121 |
| VGFB71 | GFIFRDYN | 125 | ITNDGSST | 126 | VKVYYGMDD | 127 |
| VGFB78 | SFTFYNYD | 131 | IGTSGTDM | 132 | AKDYYYYGMDV | 133 |
| VGFB400 | SFTFYNYD | 131 | IGTSGTDM | 132 | AKDYYYYGMDV | 133 |
| VGFB82 | GFDFSTYA | 137 | INSEGTIT | 138 | SSTTGTTHGMDV | 139 |

TABLE 12
LCDRs of selected anti-VEGFR1 antibodies using
IMTG delineation

| | | LCDR1 | | LCDR2 | | LCDR3 |
|---|---|---|---|---|---|---|
| | LCDR1 amino | SEQ ID | LCDR2 | SEQ ID | | SEQ ID |
| mAb | acid | NO: | amino acid | NO: | LCDR3 sequence | NO: |
| VGFB54 | RSNIGNNF | 122 | LNS | NA | ASWDDSLNGWV | 124 |
| VGFB883 | RSNIGNNF | 122 | LNS | NA | ASWDDSLNGWV | 124 |
| VGFB71 | SSNIGSNY | 128 | FNF | NA | AAWDDRVNVWV | 130 |
| VGFB78 | TSNIGNNY | 134 | YD | NA | AAWDDSLNGWV | 136 |
| VGFB400 | TSNIGNNY | 134 | YD | NA | AAWDDSLNGWV | 136 |
| VGFB82 | SSNIQNNY | 140 | FNS | NA | GTWDDSLNGWV | 142 |

NA = Not Applicable

TABLE 13
VH and VL amino acid sequence of selected anti-
VEGF1 antibodies

| | | | VH | | | VL |
|---|---|---|---|---|---|---|
| | | | SEQ | | | SEQ |
| | | VH amino acid | ID | | VL amino acid | ID |
| Antibody | VH name | sequence | NO: | VL name | sequence | NO: |
| VGFB54 | VD0000 | EVQLLESGGGSVQP | 31 | VD0000 | QSVLTQPPSAS | 32 |
| | 49673_ | GGSLRLSCTASGFNF | | 49676_ | GTPGQRVTISCS | |
| | VH | EDYGMNWVRQAPG | | VL | GSRSNIGNNFV | |
| | | KGLDWVSSINRNGD | | | NWYQQIPGTAP | |
| | | ISTYHVDSVKGRFTI | | | KLLIYLNSQRPS | |
| | | SRDNSKNTLYLQMN | | | GVP | |
| | | SLRAEDTAIYYCTK | | | DRFSGSKSGTS | |
| | | NHDYHGMDVWGQ | | | ASLAISGLQSDD | |
| | | GTTVTVSS | | | EADYYCASWD | |
| | | | | | DSLNGWVFGG | |
| | | | | | GTKLTVL | |
| VGFB883 | VD0000 | EVQLLESGGGSVQP | 33 | VD0000 | QSVLTQPPSAS | 32 |
| | 60031_ | GGSLRLSCTASGFNF | | 49676_ | GTPGQRVTISCS | |
| | VH | EDYGMNWVRQAPG | | VL | GSRSNIGNNFV | |
| | | KGLDWVSSINRQGD | | | NWYQQIPGTAP | |
| | | ISTYHVDSVKGRFTI | | | KLLIYLNSQRPS | |
| | | SRDNSKNTLYLQMN | | | GVP | |
| | | SLRAEDTAIYYCTK | | | DRFSGSKSGTS | |
| | | NHDYHGMDVWGQ | | | ASLAISGLQSDD | |
| | | GTTVTVSS | | | EADYYCASWD | |
| | | | | | DSLNGWVFGG | |
| | | | | | GTKLTVL | |
| VGFB71 | VD0000 | EVQLLESGGGLVQP | 34 | VD0000 | QSVLTQPPSAS | 35 |
| | 50424_ | GGSLRLSCEASGFIF | | 50423_ | GTPGQRVTISCS | |
| | VH | RDYNMNWVRQAPG | | VL | GGSSNIGSNYV | |
| | | KGLEWVSIITNDGSS | | | SWYQQLPGTAP | |
| | | TAY | | | KLLIYFNFQRPS | |
| | | SDSVKGRFTISRDNS | | | GVP | |
| | | KNSLYLQMNSLRAE | | | DRFSGSKSGTS | |
| | | DTAIYYCVKVYYG | | | ASLAISGLQSED | |
| | | MDDWGQGTTVTVS | | | EADYYCAAWD | |
| | | S | | | DRVNVWVFGG | |
| | | | | | GTKLTVL | |
| VGFB78 | VD0000 | EVQLLESGGGLVQP | 36 | VD0000 | QSVLTQPPSAS | 37 |
| | 50433_ | GGSLRLSCAASSFTF | | 50421_ | GTPGQRVIISCS | |
| | VH | YNYDMTWVRQAPG | | VL | GSTSNIGNNYV | |
| | | KGLEWVAGIGTSGT | | | SWYQQLPGTAP | |
| | | DMYY | | | KLLIYYDQRPS | |
| | | ADSVKGRFTISRDNS | | | GVPD | |
| | | KNTLYLQMNSLRAE | | | RFSGSKSGTSAS | |
| | | DTAIYYCAKDYYYY | | | LAISGLQSEDEA | |
| | | GMDVWGLGTTVTV | | | DYYCAAWDDS | |
| | | SS | | | LNGWVFGGGT | |
| | | | | | KLTVL | |
| VGFB400 | VD0000 | EVQLLESGGGLVQP | 38 | VD0000 | QSVLTQPPSAS | 37 |
| | 59020_ | GGSLRLSCAASSFTF | | 50421_ | GTPGQRVIISCS | |
| | VH | YNYDMTWVRQAPG | | VL | GSTSNIGNNYV | |
| | | KGLEWVAGIGTSGT | | | SWYQQLPGTAP | |
| | | DMYYADSVKGRFTI | | | KLLIYYDQRPS | |
| | | SRDNSKNTLYLQMN | | | GVPD | |
| | | SLRAEDTAIYYCAK | | | RFSGSKSGTSAS | |
| | | DYYYYGMDVWGQ | | | LAISGLQSEDEA | |
| | | GTTVTVSS | | | DYYCAAWDDS | |
| | | | | | LNGWVFGGGT | |
| | | | | | KLTVL | |
| VGFB82 | VD0000 | EVQLLESGGGLVQP | 39 | VD0000 | QSVLTQPPSAS | 40 |
| | 56903_ | GGSLRLSCEASGFDF | | 56902_ | GTPGQRVTISCS | |
| | VH | STYAMNWVRQAPG | | VL | GSSSNIQNNYV | |
| | | KGLEWVATINSEGTI | | | YWYQQLPGTA | |
| | | TSHAPAVKGRFTISR | | | PKLLIYFNSQRP | |
| | | DNSKNTAYVQMNS | | | SGVP | |
| | | LRAEDSAVYYCSST | | | DRFSGSKSGTS | |
| | | TGTTHGMDVWGRG | | | ASLAISGLQSED | |
| | | TTVTVSS | | | EADYYCGTWD | |
| | | | | | DSLNGWVFGG | |
| | | | | | GTKLTVL | |

TABLE 14
VH nucleic acid sequence of selected anti-VEGFR1 antibodies

| | | | VH |
|---|---|---|---|
| | | | SEQ |
| | | | ID |
| Antibody | VH name | VH nucleic acid sequence | NO: |
| VGFB54 | VD0000496 | GAGGTTCAGC TGCTGGAATC TGGCGGAGGA | 41 |
| | 73_VH | TCTGTTCAGC CTGGCGGCTC TCTGAGACTG | |
| | | TCTTGTACCG CCTCCGGCTT CAACTTCGAG | |
| | | GACTACGGCA TGAACTGGGT CCGACAGGCT | |
| | | CCTGGCAAAG GCCTGGATTG GGTGTCCTCC | |
| | | ATCAACCGGA ACGGCGACAT CTCCACCTAC | |
| | | CACGTGGACT CCGTGAAGGG CAGATTCACC | |
| | | ATCAGCCGGG ACAACTCCAA GAACACCCTG | |
| | | TACCTGCAGA TGAACTCCCT GAGAGCCGAG | |
| | | GACACCGCCA TCTACTACTG CACCAAGAAC | |
| | | CACGACTACC ACGGCATGGA TGTGTGGGGC | |
| | | CAAGGCACAA CAGTGACCGT TTCTTCT | |
| VGFB883 | VD0000600 | GAGGTTCAGC TGCTGGAATC TGGCGGAGGA | 43 |
| | 31_VH | TCTGTTCAGC CTGGCGGCTC TCTGAGACTG | |
| | | TCTTGTACCG CCTCCGGCTT CAACTTCGAG | |
| | | GACTACGGCA TGAACTGGGT CCGACAGGCT | |
| | | CCTGGCAAAG GCCTGGATTG GGTGTCCTCT | |
| | | ATCAACCGGC AGGGCGACAT CTCCACCTAC | |
| | | CACGTGGACT CTGTGAAGGG CAGATTCACC | |
| | | ATCAGCCGGG ACAACTCCAA GAACACCCTG | |
| | | TACCTGCAGA TGAACTCCCT GAGAGCCGAG | |
| | | GACACCGCCA TCTACTACTG CACCAAGAAC | |
| | | CACGACTACC ACGGCATGGA TGTGTGGGGA | |
| | | CAAGGCACAA CAGTGACCGT TTCTTCT | |
| VGFB71 | VD0000504 | GAGGTTCAGC TGCTGGAATC TGGCGGAGGA | 44 |
| | 24_VH | TTGGTTCAGC CTGGCGGCTC TCTGAGACTG | |
| | | TCTTGCGAGG CCTCTGGCTT CATCTTCCGG | |
| | | GACTACAACA TGAACTGGGT CCGACAGGCT | |
| | | CCTGGCAAAG GCCTGGAATG GGTGTCCATC | |
| | | ATCACCAACG ACGGCTCCTC CACCGCCTAC | |
| | | TCCGATTCTG TGAAGGGCAG ATTCACCATC | |
| | | AGCCGGGACA ACTCCAAGAA CTCCCTGTAC | |
| | | CTGCAGATGA ACAGCCTGAG AGCCGAGGAC | |
| | | ACCGCCATCT ACTACTGCGT GAAGGTGTAC | |
| | | TACGGCATGG ACGATTGGGG CCAGGGCACC | |
| | | ACAGTGACAG TCTCTTCT | |
| VGFB78 | VD0000504 | GAAGTTCAGC TTTTGGAGTC CGGTGGGGGA | 46 |
| | 33_VH | TTGGTTCAGC CAGGGGGAAG TCTGCGATTG | |
| | | TCATGTGCAG CATCCTCCTT CACATTCTAT | |
| | | AATTATGACA TGACATGGGT ACGGCAGGCA | |
| | | CCAGGAAAAG GACTGGAGTG GGTTGCTGGG | |
| | | ATTGGGACAT CAGGAACTGA TATGTATTAC | |
| | | GCAGATTCAG TAAAGGGTCG CTTTACAATA | |
| | | AGCCGCGATA ATAGTAAAAA TACATTGTAT | |
| | | CTTCAAATGA ACTCACTCAG AGCCGAGGAC | |
| | | ACTGCTATTT ACTACTGCGC TAAAGATTAC | |
| | | TATTATTACG GGATGGACGT ATGGGGCCTT | |
| | | GGCACAACTG TTACCGTCTC AAGC | |
| VGFB400 | VD0000590 | GAGGTTCAGC TGCTGGAATC TGGCGGAGGA | 48 |
| | 20_VH | TTGGTTCAGC CTGGCGGTTC TCTGAGACTG | |
| | | TCTTGTGCCG CCTCCTCCTT CACCTTCTAC | |
| | | AACTACGACA TGACCTGGGT CCGACAGGCC | |
| | | CCTGGCAAAG GATTGGAATG GGTGGCCGGC | |
| | | ATCGGCACCT CTGGCACCGA TATGTACTAC | |
| | | GCCGACTCCG TGAAGGGCAG ATTCACCATC | |
| | | TCTCGGGACA ACTCCAAGAA CACCCTGTAC | |
| | | CTGCAGATGA ACTCCCTGAG AGCCGAGGAC | |
| | | ACCGCCATCT ACTACTGCGC CAAGGATTAC | |
| | | TACTACTACG GCATGGACGT GTGGGGCCAA | |
| | | GGCACAACAG TGACAGTCTC TTCT | |
| VGFB82 | VD0000569 | GAGGTTCAGC TGCTGGAATC TGGCGGAGGA | 49 |
| | 03_VH | TTGGTTCAGC CTGGCGGCTC TCTGAGACTG | |
| | | TCTTGCGAGG CCTCTGGCTT CGACTTCTCC | |
| | | ACCTACGCCA TGAACTGGGT CCGACAGGCT | |
| | | CCTGGCAAAG GCCTGGAATG GGTCGCCACC | |
| | | ATCAATTCCG AGGGCACCAT CACCTCTCAC | |
| | | GCCCCTGCTG TGAAGGGCAG ATTCACCATC | |
| | | TCTCGGGACA ACTCCAAGAA CACCGCCTAC | |
| | | GTGCAGATGA ACTCCCTGAG AGCCGAGGAC | |
| | | TCCGCCGTGT ACTACTGTTC TTCTACCACC | |
| | | GGCACCACAC ACGGCATGGA TGTGTGGGGA | |
| | | CGCGGCACCA CAGTGACCGT TTCTTCT | |

TABLE 15
VL nucleic acid sequence of selected anti-
VEGFR1 antibodies

| | | | VL |
|---|---|---|---|
| | | | SEQ |
| Antibody | VL name | VL nucleic acid sequence | ID NO: |
| VGFB54 | VD00 | CAGTCTGTGC TGACCCAGCC TCCTTCTGCT | 42 |
| | 00496 | TCTGGCACAC CTGGCCAGAG AGTGACCATC | |
| | 76_VL | TCCTGTTCCG GCTCTCGGTC CAACATCGGC | |
| | | AACAACTTCG TGAACTGGTA TCAGCAGATC | |
| | | CCCGGCACCG CTCCTAAGCT GCTGATCTAC | |
| | | CTGAACTCCC AGCGGCCTTC TGGCGTGCCC | |
| | | GATAGATTCT CCGGCTCTAA GTCTGGCACC | |
| | | TCTGCCAGCC TGGCTATCTC TGGCCTGCAG | |
| | | TCTGATGACG AGGCCGACTA CTACTGCGCC | |
| | | TCCTGGGACG ATTCTCTGAA CGGCTGGGTT | |
| | | TTCGGCGGAG GCACCAAACT GACAGTGCTG | |
| VGFB883 | VD00 | CAGTCTGTGC TGACCCAGCC TCCTTCTGCT | 42 |
| | 00496 | TCTGGCACAC CTGGCCAGAG AGTGACCATC | |
| | 76_VL | TCCTGTTCCG GCTCTCGGTC CAACATCGGC | |
| | | AACAACTTCG TGAACTGGTA TCAGCAGATC | |
| | | CCCGGCACCG CTCCTAAGCT GCTGATCTAC | |
| | | CTGAACTCCC AGCGGCCTTC TGGCGTGCCC | |
| | | GATAGATTCT CCGGCTCTAA GTCTGGCACC | |
| | | TCTGCCAGCC TGGCTATCTC TGGCCTGCAG | |
| | | TCTGATGACG AGGCCGACTA CTACTGCGCC | |
| | | TCCTGGGACG ATTCTCTGAA CGGCTGGGTT | |
| | | TTCGGCGGAG GCACCAAACT GACAGTGCTG | |
| VGFB71 | VD00 | CAGTCTGTGC TGACCCAGCC TCCTTCTGCT | 45 |
| | 00504 | TCTGGCACAC CTGGCCAGAG AGTGACCATC | |
| | 23_VL | TCTTGCTCTG GCGGCTCCTC CAACATCGGC | |
| | | TCCAATTACG TGTCCTGGTA TCAGCAGCTG | |
| | | CCCGGCACAG CTCCCAAACT GCTGATCTAC | |
| | | TTCAACTTCC AGCGGCCTTC TGGCGTGCCC | |
| | | GACAGATTCT CTGGCTCTAA GTCTGGCACC | |
| | | TCTGCCAGCC TGGCTATCTC TGGACTGCAG | |
| | | AGCGAGGACG AGGCCGACTA CTATTGTGCC | |
| | | GCCTGGGACG ACAGAGTGAA CGTGTGGGTT | |
| | | TTCGGCGGAG GCACCAAGCT GACAGTTCTG | |
| VGFB78 | VD00 | CAGTCTGTGC TGACCCAGCC TCCTTCTGCT | 47 |
| | 00504 | TCTGGCACAC CTGGCCAGAG AGTGATCATC | |
| | 21_VL | TCCTGCTCCG GCTCCACCTC CAACATCGGC | |
| | | AACAACTACG TGTCCTGGTA TCAGCAGCTG | |
| | | CCCGGCACAG CTCCCAAACT GCTGATCTAC | |
| | | TACGACCAGC GGCCTTCTGG CGTGCCCGAT | |
| | | AGATTCTCCG GCTCTAAGTC TGGCACCTCT | |
| | | GCCAGCCTGG CTATCTCTGG ACTGCAGAGC | |
| | | GAGGACGAGG CCGACTACTA TTGTGCCGCC | |
| | | TGGGACGATT CTCTGAACGG CTGGGTTTTC | |
| | | GGCGGAGGCA CCAAACTGAC AGTGCTG | |
| VGFB400 | VD00 | CAGTCTGTGC TGACCCAGCC TCCTTCTGCT | 47 |
| | 00504 | TCTGGCACAC CTGGCCAGAG AGTGATCATC | |
| | 21_VL | TCCTGCTCCG GCTCCACCTC CAACATCGGC | |
| | | AACAACTACG TGTCCTGGTA TCAGCAGCTG | |
| | | CCCGGCACAG CTCCCAAACT GCTGATCTAC | |
| | | TACGACCAGC GGCCTTCTGG CGTGCCCGAT | |
| | | AGATTCTCCG GCTCTAAGTC TGGCACCTCT | |
| | | GCCAGCCTGG CTATCTCTGG ACTGCAGAGC | |
| | | GAGGACGAGG CCGACTACTA TTGTGCCGCC | |
| | | TGGGACGATT CTCTGAACGG CTGGGTTTTC | |
| | | GGCGGAGGCA CCAAACTGAC AGTGCTG | |
| VGFB82 | VD00 | CAGTCTGTGC TGACCCAGCC TCCTTCTGCT | 50 |
| | 00569 | TCTGGCACAC CTGGCCAGAG AGTGACCATC | |
| | 02_VL | TCTTGCTCCG GCTCCTCCAG CAACATCCAG | |
| | | AACAACTACG TGTACTGGTA TCAGCAGCTG | |
| | | CCCGGCACCG CTCCTAAACT GCTGATCTAC | |
| | | TTCAACTCCC AGCGGCCTTC TGGCGTGCCC | |
| | | GATAGATTCT CCGGCTCTAA GTCTGGCACC | |
| | | TCTGCCAGCC TGGCTATCTC TGGACTGCAG | |
| | | AGCGAGGACG AGGCCGACTA CTATTGTGGC | |
| | | ACCTGGGACG ATTCCCTGAA CGGCTGGGTT | |
| | | TTCGGCGGAG GCACAAAGCT GACAGTGCTG | |

TABLE 16
HC amino acid sequence of selected anti-VEGFR1 antibodies

| | | HC | |
|---|---|---|---|
| | HC | PROTEIN | |
| | PEPTIDE | SEQ ID | |
| Antibody | ID | NO: | HC AMINO ACID SEQUENCE |
| VGFB54 | DCH000 | 51 | EVQLLESGGGSVQPGGSLRLSCTASGFNFEDYG |
| | 001577 | | MNWVRQAPGKGLDWVSSINRNGDISTYHVDSV |
| | | | KGRFTISRDNSKNTLYLQMNSLRAEDTAIYYCTK |
| | | | NHDYHGMDVWGQGTTVTVSSASTKGPSVFPLAP |
| | | | SSKSTSGGTAALGCLVKDYFPEPVTVSWNSGALT |
| | | | SGVHTFPAVLQSSGLYSLSSVVTVPSSSLGTQTYI |
| | | | CNVNHKPSNTKVDKKVEPKSCDKTHTCPPCPAP |
| | | | EAAGGPSVFLFPPKPKDTLMISRTPEVTCVVVSVS |
| | | | HEDPEVKFNWYVDGVEVHNAKTKPREEQYNST |
| | | | YRVVSVLTVLHQDWLNGKEYKCKVSNKALPAPI |
| | | | EKTISKAKGQPREPQVYTLPPSREEMTKNQVSLT |
| | | | CLVKGFYPSDIAVEWESNGQPENNYKTTPPVLDS |
| | | | DGSFFLYSKLTVDKSRWQQGNVFSCSVMHEALH |
| | | | NHYTQKSLSLSPGK |
| VGFB883 | DCH000 | 53 | EVQLLESGGGSVQPGGSLRLSCTASGFNFEDYG |
| | 013229 | | MNWVRQAPGKGLDWVSSINRQGDISTYHVDSV |
| | | | KGRFTISRDNSKNTLYLQMNSLRAEDTAIYYCTK |
| | | | NHDYHGMDVWGQGTTVTVSSASTKGPSVFPLAP |
| | | | SSKSTSGGTAALGCLVKDYFPEPVTVSWNSGALT |
| | | | SGVHTFPAVLQSSGLYSLSSVVTVPSSSLGTQTYI |
| | | | CNVNHKPSNTKVDKKVEPKSCDKTHTCPPCPAP |
| | | | EAAGGPSVFLFPPKPKDTLMISRTPEVTCVVVSVS |
| | | | HEDPEVKFNWYVDGVEVHNAKTKPREEQYNST |
| | | | YRVVSVLTVLHQDWLNGKEYKCKVSNKALPAPI |
| | | | EKTISKAKGQPREPQVYTLPPSREEMTKNQVSLT |
| | | | CLVKGFYPSDIAVEWESNGQPENNYKTTPPVLDS |
| | | | DGSFFLYSKLTVDKSRWQQGNVFSCSVMHEALH |
| | | | NHYTQKSLSLSPGK |
| VGFB71 | DCH000 | 54 | EVQLLESGGGLVQPGGSLRLSCEASGFIFRDYNM |
| | 001642 | | NWVRQAPGKGLEWVSIITNDGSSTAYSDSVKGR |
| | | | FTISRDNSKNSLYLQMNSLRAEDTAIYYCVKVYY |
| | | | GMDDWGQGTTVTVSSASTKGPSVFPLAPSSKSTS |
| | | | GGTAALGCLVKDYFPEPVTVSWNSGALTSGVHT |
| | | | FPAVLQSSGLYSLSSVVTVPSSSLGTQTYICNVNH |
| | | | KPSNTKVDKKVEPKSCDKTHTCPPCPAPEAAGGP |
| | | | SVFLFPPKPKDTLMISRTPEVTCVVVSVSHEDPEV |
| | | | KFNWYVDGVEVHNAKTKPREEQYNSTYRVVSV |
| | | | LTVLHQDWLNGKEYKCKVSNKALPAPIEKTISK |
| | | | AKGQPREPQVYTLPPSREEMTKNQVSLTCLVKG |
| | | | FYPSDIAVEWESNGQPENNYKTTPPVLDSDGSFF |
| | | | LYSKLTVDKSRWQQGNVFSCSVMHEALHNHYT |
| | | | QKSLSLSPGK |
| VGFB78 | DCH000 | 56 | EVQLLESGGGLVQPGGSLRLSCAASSFTFYNYD |
| | 001676 | | MTWVRQAPGKGLEWVAGIGTSGTDMYYADSV |
| | | | KGRFTISRDNSKNTLYLQMNSLRAEDTAIYYCAK |
| | | | DYYYYGMDVWGLGTTVTVSSASTKGPSVFPLAP |
| | | | SSKSTSGGTAALGCLVKDYFPEPVTVSWNSGALT |
| | | | SGVHTFPAVLQSSGLYSLSSVVTVPSSSLGTQTYI |
| | | | CNVNHKPSNTKVDKKVEPKSCDKTHTCPPCPAP |
| | | | EAAGGPSVFLFPPKPKDTLMISRTPEVTCVVVSVS |
| | | | HEDPEVKFNWYVDGVEVHNAKTKPREEQYNST |
| | | | YRVVSVLTVLHQDWLNGKEYKCKVSNKALPAPI |
| | | | EKTISKAKGQPREPQVYTLPPSREEMTKNQVSLT |
| | | | CLVKGFYPSDIAVEWESNGQPENNYKTTPPVLDS |
| | | | DGSFFLYSKLTVDKSRWQQGNVFSCSVMHEALH |
| | | | NHYTQKSLSLSPGK |
| VGFB400 | DCH000 | 58 | EVQLLESGGGLVQPGGSLRLSCAASSFTFYNYD |
| | 007928 | | MTWVRQAPGKGLEWVAGIGTSGTDMYYADSV |
| | | | KGRFTISRDNSKNTLYLQMNSLRAEDTAIYYCAK |
| | | | DYYYYGMDVWGQGTTVTVSSASTKGPSVFPLAP |
| | | | SSKSTSGGTAALGCLVKDYFPEPVTVSWNSGALT |
| | | | SGVHTFPAVLQSSGLYSLSSVVTVPSSSLGTQTYI |
| | | | CNVNHKPSNTKVDKKVEPKSCDKTHTCPPCPAP |
| | | | EAAGGPSVFLFPPKPKDTLMISRTPEVTCVVVSVS |
| | | | HEDPEVKFNWYVDGVEVHNAKTKPREEQYNST |
| | | | YRVVSVLTVLHQDWLNGKEYKCKVSNKALPAPI |
| | | | EKTISKAKGQPREPQVYTLPPSREEMTKNQVSLT |
| | | | CLVKGFYPSDIAVEWESNGQPENNYKTTPPVLDS |
| | | | DGSFFLYSKLTVDKSRWQQGNVFSCSVMHEALH |
| | | | NHYTQKSLSLSPGK |
| VGFB82 | DCH000 | 59 | EVQLLESGGGLVQPGGSLRLSCEASGFDFSTYAM |
| | 005623 | | NWVRQAPGKGLEWVATINSEGTITSHAPAVKGR |
| | | | FTISRDNSKNTAYVQMNSLRAEDSAVYYCSSTTG |
| | | | TTHGMDVWGRGTTVTVSSASTKGPSVFPLAPSS |
| | | | KSTSGGTAALGCLVKDYFPEPVTVSWNSGALTS |
| | | | GVHTFPAVLQSSGLYSLSSVVTVPSSSLGTQTYIC |
| | | | NVNHKPSNTKVDKKVEPKSCDKTHTCPPCPAPE |
| | | | AAGGPSVFLFPPKPKDTLMISRTPEVTCVVVSVS |
| | | | HEDPEVKFNWYVDGVEVHNAKTKPREEQYNST |
| | | | YRVVSVLTVLHQDWLNGKEYKCKVSNKALPAPI |
| | | | EKTISKAKGQPREPQVYTLPPSREEMTKNQVSLT |
| | | | CLVKGFYPSDIAVEWESNGQPENNYKTTPPVLDS |
| | | | DGSFFLYSKLTVDKSRWQQGNVFSCSVMHEALH |
| | | | NHYTQKSLSLSPGK |

TABLE 17
LC amino acid sequences of selected anti-VEGFR1 antibodies.

| | | LC | |
|---|---|---|---|
| | LC | PROTEIN | |
| | PEPTIDE | SEQ ID | |
| ANTIBODY | ID | NO: | LC AMINO ACID SEQUENCE |
| VGFB54 | DCH0000 | 52 | QSVLTQPPSASGTPGQRVTISCSGSRSNIGNNFVN |
| | 01057 | | WYQQIPGTAPKLLIYLNSQRPSGVPDRFSGSKSGT |
| | | | SASLAISGLQSDDEADYYCASWDDSLNGWVFGG |
| | | | GTKLTVLGQPKAAPSVTLFPPSSEELQANKATLVC |
| | | | LISDFYPGAVTVAWKADSSPVKAGVETTTPSKQS |
| | | | NNKYAASSYLSLTPEQWKSHRSYSCQVTHEGSTV |
| | | | EKTVAPTECS |
| VGFB883 | DCH0000 | 52 | QSVLTQPPSASGTPGQRVTISCSGSRSNIGNNFVN |
| | 01057 | | WYQQIPGTAPKLLIYLNSQRPSGVPDRFSGSKSGT |
| | | | SASLAISGLQSDDEADYYCASWDDSLNGWVFGG |
| | | | GTKLTVLGQPKAAPSVTLFPPSSEELQANKATLVC |
| | | | LISDFYPGAVTVAWKADSSPVKAGVETTTPSKQS |
| | | | NNKYAASSYLSLTPEQWKSHRSYSCQVTHEGSTV |
| | | | EKTVAPTECS |
| VGFB71 | DCH0000 | 55 | QSVLTQPPSASGTPGQRVTISCSGGSSNIGSNYVS |
| | 01643 | | WYQQLPGTAPKLLIYFNFQRPSGVPDRFSGSKSGT |
| | | | SASLAISGLQSEDEADYYCAAWDDRVNVWVFGG |
| | | | GTKLTVLGQPKAAPSVTLFPPSSEELQANKATLVC |
| | | | LISDFYPGAVTVAWKADSSPVKAGVETTTPSKQS |
| | | | NNKYAASSYLSLTPEQWKSHRSYSCQVTHEGSTV |
| | | | EKTVAPTECS |
| VGFB78 | DCH0000 | 57 | QSVLTQPPSASGTPGQRVIISCSGSTSNIGNNYVSW |
| | 01641 | | YQQLPGTAPKLLIYYDQRPSGVPDRFSGSKSGTSA |
| | | | SLAISGLQSEDEADYYCAAWDDSLNGWVFGGGT |
| | | | KLTVLGQPKAAPSVTLFPPSSEELQANKATLVCLI |
| | | | SDFYPGAVTVAWKADSSPVKAGVETTTPSKQSNN |
| | | | KYAASSYLSLTPEQWKSHRSYSCQVTHEGSTVEK |
| | | | TVAPTECS |
| VGFB400 | DCH0000 | 57 | QSVLTQPPSASGTPGQRVIISCSGSTSNIGNNYVSW |
| | 01641 | | YQQLPGTAPKLLIYYDQRPSGVPDRFSGSKSGTSA |
| | | | SLAISGLQSEDEADYYCAAWDDSLNGWVFGGGT |
| | | | KLTVLGQPKAAPSVTLFPPSSEELQANKATLVCLI |
| | | | SDFYPGAVTVAWKADSSPVKAGVETTTPSKQSNN |
| | | | KYAASSYLSLTPEQWKSHRSYSCQVTHEGSTVEK |
| | | | TVAPTECS |
| VGFB82 | DCH0000 | 60 | QSVLTQPPSASGTPGQRVTISCSGSSSNIQNNYVY |
| | 05624 | | WYQQLPGTAPKLLIYFNSQRPSGVPDRFSGSKSGT |
| | | | SASLAISGLQSEDEADYYCGTWDDSLNGWVFGG |
| | | | GTKLTVLGQPKAAPSVTLFPPSSEELQANKATLVC |
| | | | LISDFYPGAVTVAWKADSSPVKAGVETTTPSKQS |
| | | | NNKYAASSYLSLTPEQWKSHRSYSCQVTHEGSTV |
| | | | EKTVAPTECS |

TABLE 18
HC nucleotide sequences of selected anti-VEGFR1 antibodies.

| | HC | |
|---|---|---|
| | NUCLEO- | |
| | TIDE | |
| | SEQ ID | |
| Antibody | NO: | HC NUCLEIC ACID SEQUENCE |
| VGFB54 | 61 | GAGGTTCAGCTGCTGGAATCTGGCGGAGGATCTGTTCAGC |
| | | CTGGCGGCTCTCTGAGACTGTCTTGTACCGCCTCCGGCTTC |
| | | AACTTCGAGGACTACGGCATGAACTGGGTCCGACAGGCTC |
| | | CTGGCAAAGGCCTGGATTGGGTGTCCTCCATCAACCGGAA |
| | | CGGCGACATCTCCACCTACCACGTGGACTCCGTGAAGGGC |
| | | AGATTCACCATCAGCCGGGACAACTCCAAGAACACCCTGT |
| | | ACCTGCAGATGAACTCCCTGAGAGCCGAGGACACCGCCAT |
| | | CTACTACTGCACCAAGAACCACGACTACCACGGCATGGAT |
| | | GTGTGGGGCCAAGGCACAACAGTGACCGTTTCTTCTGCCTC |
| | | CACCAAGGGCCCATCGGTCTTCCCCCTGGCACCCTCCTCCA |
| | | AGAGCACCTCTGGGGGCACAGCGGCCCTGGGCTGCCTGGT |
| | | CAAGGACTACTTCCCCGAACCGGTGACGGTGTCGTGGAAC |
| | | TCAGGCGCCCTGACCAGCGGCGTGCACACCTTCCCGGCTGT |
| | | CCTACAGTCCTCAGGACTCTACTCCCTCAGCAGCGTGGTGA |
| | | CCGTGCCCTCCAGCAGCTTGGGCACCCAGACCTACATCTGC |
| | | AACGTGAATCACAAGCCCAGCAACACCAAGGTGGACAAGA |
| | | AAGTTGAGCCCAAATCTTGTGACAAAACTCACACATGCCC |
| | | ACCGTGCCCAGCACCTGAAGCCGCCGGGGGACCGTCAGTC |
| | | TTCCTCTTCCCCCCAAAACCCAAGGACACCCTCATGATCTC |
| | | CCGGACCCCTGAGGTCACATGCGTGGTGGTGAGCGTGAGC |
| | | CACGAAGACCCTGAGGTCAAGTTCAACTGGTACGTGGACG |
| | | GCGTGGAGGTGCATAATGCCAAGACAAAGCCGCGGGAGG |
| | | AGCAGTACAACAGCACGTACCGTGTGGTCAGCGTCCTCAC |
| | | CGTCCTGCACCAGGACTGGCTGAATGGCAAGGAGTACAAG |
| | | TGCAAGGTGTCGAACAAAGCCCTCCCAGCCCCCATCGAGA |
| | | AAACCATCTCCAAAGCCAAAGGGCAGCCCCGAGAACCACA |
| | | GGTGTACACCCTGCCCCCATCCCGGGAGGAGATGACCAAG |
| | | AACCAGGTCAGCCTGACCTGCCTGGTCAAAGGCTTCTATCC |
| | | CAGCGACATCGCCGTGGAGTGGGAGAGCAATGGGCAGCCG |
| | | GAGAACAACTACAAGACCACGCCTCCCGTGCTGGACTCCG |
| | | ACGGCTCCTTCTTCCTCTACAGCAAGCTCACCGTGGACAAG |
| | | AGCAGATGGCAGCAGGGGAACGTCTTCTCATGCTCCGTGA |
| | | TGCATGAGGCTCTGCACAACCACTACACGCAGAAGTCTCTC |
| | | TCCCTGTCTCCGGGAAAA |
| VGFB883 | 63 | GAGGTTCAGCTGCTGGAATCTGGCGGAGGATCTGTTCAGC |
| | | CTGGCGGCTCTCTGAGACTGTCTTGTACCGCCTCCGGCTTC |
| | | AACTTCGAGGACTACGGCATGAACTGGGTCCGACAGGCTC |
| | | CTGGCAAAGGCCTGGATTGGGTGTCCTCTATCAACCGGCA |
| | | GGGCGACATCTCCACCTACCACGTGGACTCTGTGAAGGGC |
| | | AGATTCACCATCAGCCGGGACAACTCCAAGAACACCCTGT |
| | | ACCTGCAGATGAACTCCCTGAGAGCCGAGGACACCGCCAT |
| | | CTACTACTGCACCAAGAACCACGACTACCACGGCATGGAT |
| | | GTGTGGGGACAAGGCACAACAGTGACCGTTTCTTCTGCCTC |
| | | CACCAAGGGCCCATCGGTCTTCCCCCTGGCACCCTCCTCCA |
| | | AGAGCACCTCTGGGGGCACAGCGGCCCTGGGCTGCCTGGT |
| | | CAAGGACTACTTCCCCGAACCGGTGACGGTGTCGTGGAAC |
| | | TCAGGCGCCCTGACCAGCGGCGTGCACACCTTCCCGGCTGT |
| | | CCTACAGTCCTCAGGACTCTACTCCCTCAGCAGCGTGGTGA |
| | | CCGTGCCCTCCAGCAGCTTGGGCACCCAGACCTACATCTGC |
| | | AACGTGAATCACAAGCCCAGCAACACCAAGGTGGACAAGA |
| | | AAGTTGAGCCCAAATCTTGTGACAAAACTCACACATGCCC |
| | | ACCGTGCCCAGCACCTGAAGCCGCCGGGGGACCGTCAGTC |
| | | TTCCTCTTCCCCCCAAAACCCAAGGACACCCTCATGATCTC |
| | | CCGGACCCCTGAGGTCACATGCGTGGTGGTGAGCGTGAGC |
| | | CACGAAGACCCTGAGGTCAAGTTCAACTGGTACGTGGACG |
| | | GCGTGGAGGTGCATAATGCCAAGACAAAGCCGCGGGAGG |
| | | AGCAGTACAACAGCACGTACCGTGTGGTCAGCGTCCTCAC |
| | | CGTCCTGCACCAGGACTGGCTGAATGGCAAGGAGTACAAG |
| | | TGCAAGGTGTCGAACAAAGCCCTCCCAGCCCCCATCGAGA |
| | | AAACCATCTCCAAAGCCAAAGGGCAGCCCCGAGAACCACA |
| | | GGTGTACACCCTGCCCCCATCCCGGGAGGAGATGACCAAG |
| | | AACCAGGTCAGCCTGACCTGCCTGGTCAAAGGCTTCTATCC |
| | | CAGCGACATCGCCGTGGAGTGGGAGAGCAATGGGCAGCCG |
| | | GAGAACAACTACAAGACCACGCCTCCCGTGCTGGACTCCG |
| | | ACGGCTCCTTCTTCCTCTACAGCAAGCTCACCGTGGACAAG |
| | | AGCAGATGGCAGCAGGGGAACGTCTTCTCATGCTCCGTGA |
| | | TGCATGAGGCTCTGCACAACCACTACACGCAGAAGTCTCTC |
| | | TCCCTGTCTCCGGGAAAA |
| VGFB71 | 64 | GAGGTTCAGCTGCTGGAATCTGGCGGAGGATTGGTTCAGC |
| | | CTGGCGGCTCTCTGAGACTGTCTTGCGAGGCCTCTGGCTTC |
| | | ATCTTCCGGGACTACAACATGAACTGGGTCCGACAGGCTC |
| | | CTGGCAAAGGCCTGGAATGGGTGTCCATCATCACCAACGA |
| | | CGGCTCCTCCACCGCCTACTCCGATTCTGTGAAGGGCAGAT |
| | | TCACCATCAGCCGGGACAACTCCAAGAACTCCCTGTACCTG |
| | | CAGATGAACAGCCTGAGAGCCGAGGACACCGCCATCTACT |
| | | ACTGCGTGAAGGTGTACTACGGCATGGACGATTGGGGCCA |
| | | GGGCACCACAGTGACAGTCTCTTCTGCCTCCACCAAGGGCC |
| | | CATCGGTCTTCCCCCTGGCACCCTCCTCCAAGAGCACCTCT |
| | | GGGGGCACAGCGGCCCTGGGCTGCCTGGTCAAGGACTACT |
| | | TCCCCGAACCGGTGACGGTGTCGTGGAACTCAGGCGCCCT |
| | | GACCAGCGGCGTGCACACCTTCCCGGCTGTCCTACAGTCCT |
| | | CAGGACTCTACTCCCTCAGCAGCGTGGTGACCGTGCCCTCC |
| | | AGCAGCTTGGGCACCCAGACCTACATCTGCAACGTGAATC |
| | | ACAAGCCCAGCAACACCAAGGTGGACAAGAAAGTTGAGCC |
| | | CAAATCTTGTGACAAAACTCACACATGCCCACCGTGCCCA |
| | | GCACCTGAAGCCGCCGGGGGACCGTCAGTCTTCCTCTTCCC |
| | | CCCAAAACCCAAGGACACCCTCATGATCTCCCGGACCCCT |
| | | GAGGTCACATGCGTGGTGGTGAGCGTGAGCCACGAAGACC |
| | | CTGAGGTCAAGTTCAACTGGTACGTGGACGGCGTGGAGGT |
| | | GCATAATGCCAAGACAAAGCCGCGGGAGGAGCAGTACAA |
| | | CAGCACGTACCGTGTGGTCAGCGTCCTCACCGTCCTGCACC |
| | | AGGACTGGCTGAATGGCAAGGAGTACAAGTGCAAGGTGTC |
| | | GAACAAAGCCCTCCCAGCCCCCATCGAGAAAACCATCTCC |
| | | AAAGCCAAAGGGCAGCCCCGAGAACCACAGGTGTACACCC |
| | | TGCCCCCATCCCGGGAGGAGATGACCAAGAACCAGGTCAG |
| | | CCTGACCTGCCTGGTCAAAGGCTTCTATCCCAGCGACATCG |
| | | CCGTGGAGTGGGAGAGCAATGGGCAGCCGGAGAACAACT |
| | | ACAAGACCACGCCTCCCGTGCTGGACTCCGACGGCTCCTTC |
| | | TTCCTCTACAGCAAGCTCACCGTGGACAAGAGCAGATGGC |
| | | AGCAGGGGAACGTCTTCTCATGCTCCGTGATGCATGAGGCT |
| | | CTGCACAACCACTACACGCAGAAGTCTCTCTCCCTGTCTCC |
| | | GGGAAAA |
| VGFB78 | 66 | GAAGTTCAGCTTTTGGAGTCCGGTGGGGGATTGGTTCAGCC |
| | | AGGGGGAAGTCTGCGATTGTCATGTGCAGCATCCTCCTTCA |
| | | CATTCTATAATTATGACATGACATGGGTACGGCAGGCACC |
| | | AGGAAAAGGACTGGAGTGGGTTGCTGGGATTGGGACATCA |
| | | GGAACTGATATGTATTACGCAGATTCAGTAAAGGGTCGCTT |
| | | TACAATAAGCCGCGATAATAGTAAAAATACATTGTATCTTC |
| | | AAATGAACTCACTCAGAGCCGAGGACACTGCTATTTACTA |
| | | CTGCGCTAAAGATTACTATTATTACGGGATGGACGTATGGG |
| | | GCCTTGGCACAACTGTTACCGTCTCAAGCGCCTCCACCAAG |
| | | GGCCCATCGGTCTTCCCCCTGGCACCCTCCTCCAAGAGCAC |
| | | CTCTGGGGGCACAGCGGCCCTGGGCTGCCTGGTCAAGGAC |
| | | TACTTCCCCGAACCGGTGACGGTGTCGTGGAACTCAGGCG |
| | | CCCTGACCAGCGGCGTGCACACCTTCCCGGCTGTCCTACAG |
| | | TCCTCAGGACTCTACTCCCTCAGCAGCGTGGTGACCGTGCC |
| | | CTCCAGCAGCTTGGGCACCCAGACCTACATCTGCAACGTG |
| | | AATCACAAGCCCAGCAACACCAAGGTGGACAAGAAAGTTG |
| | | AGCCCAAATCTTGTGACAAAACTCACACATGCCCACCGTG |
| | | CCCAGCACCTGAAGCCGCCGGGGGACCGTCAGTCTTCCTCT |
| | | TCCCCCCAAAACCCAAGGACACCCTCATGATCTCCCGGACC |
| | | CCTGAGGTCACATGCGTGGTGGTGAGCGTGAGCCACGAAG |
| | | ACCCTGAGGTCAAGTTCAACTGGTACGTGGACGGCGTGGA |
| | | GGTGCATAATGCCAAGACAAAGCCGCGGGAGGAGCAGTAC |
| | | AACAGCACGTACCGTGTGGTCAGCGTCCTCACCGTCCTGCA |
| | | CCAGGACTGGCTGAATGGCAAGGAGTACAAGTGCAAGGTC |
| | | TCCAACAAAGCCCTCCCAGCCCCCATCGAGAAAACCATCT |
| | | CCAAAGCCAAAGGGCAGCCCCGAGAACCACAGGTGTACAC |
| | | CCTGCCCCCATCCCGGGAGGAGATGACCAAGAACCAGGTC |
| | | AGCCTGACCTGCCTGGTCAAAGGCTTCTATCCCAGCGACAT |
| | | CGCCGTGGAGTGGGAGAGCAATGGGCAGCCGGAGAACAA |
| | | CTACAAGACCACGCCTCCCGTGCTGGACTCCGACGGCTCCT |
| | | TCTTCCTCTACAGCAAGCTCACCGTGGACAAGAGCAGATG |
| | | GCAGCAGGGGAACGTCTTCTCATGCTCCGTGATGCATGAG |
| | | GCTCTGCACAACCACTACACGCAGAAGAGCCTCTCCCTGTC |
| | | TCCGGGTAAA |
| VGFB400 | 68 | GAGGTTCAGCTGCTGGAATCTGGCGGAGGATTGGTTCAGC |
| | | CTGGCGGTTCTCTGAGACTGTCTTGTGCCGCCTCCTCCTTCA |
| | | CCTTCTACAACTACGACATGACCTGGGTCCGACAGGCCCCT |
| | | GGCAAAGGATTGGAATGGGTGGCCGGCATCGGCACCTCTG |
| | | GCACCGATATGTACTACGCCGACTCCGTGAAGGGCAGATT |
| | | CACCATCTCTCGGGACAACTCCAAGAACACCCTGTACCTGC |
| | | AGATGAACTCCCTGAGAGCCGAGGACACCGCCATCTACTA |
| | | CTGCGCCAAGGATTACTACTACTACGGCATGGACGTGTGG |
| | | GGCCAAGGCACAACAGTGACAGTCTCTTCTGCCTCCACCA |
| | | AGGGCCCATCGGTCTTCCCCCTGGCACCCTCCTCCAAGAGC |
| | | ACCTCTGGGGGCACAGCGGCCCTGGGCTGCCTGGTCAAGG |
| | | ACTACTTCCCCGAACCGGTGACGGTGTCGTGGAACTCAGG |
| | | CGCCCTGACCAGCGGCGTGCACACCTTCCCGGCTGTCCTAC |
| | | AGTCCTCAGGACTCTACTCCCTCAGCAGCGTGGTGACCGTG |
| | | CCCTCCAGCAGCTTGGGCACCCAGACCTACATCTGCAACGT |
| | | GAATCACAAGCCCAGCAACACCAAGGTGGACAAGAAAGTT |
| | | GAGCCCAAATCTTGTGACAAAACTCACACATGCCCACCGT |
| | | GCCCAGCACCTGAAGCCGCCGGGGGACCGTCAGTCTTCCT |
| | | CTTCCCCCCAAAACCCAAGGACACCCTCATGATCTCCCGGA |
| | | CCCCTGAGGTCACATGCGTGGTGGTGAGCGTGAGCCACGA |
| | | AGACCCTGAGGTCAAGTTCAACTGGTACGTGGACGGCGTG |
| | | GAGGTGCATAATGCCAAGACAAAGCCGCGGGAGGAGCAG |
| | | TACAACAGCACGTACCGTGTGGTCAGCGTCCTCACCGTCCT |
| | | GCACCAGGACTGGCTGAATGGCAAGGAGTACAAGTGCAAG |
| | | GTGTCGAACAAAGCCCTCCCAGCCCCCATCGAGAAAACCA |
| | | TCTCCAAAGCCAAAGGGCAGCCCCGAGAACCACAGGTGTA |
| | | CACCCTGCCCCCATCCCGGGAGGAGATGACCAAGAACCAG |
| | | GTCAGCCTGACCTGCCTGGTCAAAGGCTTCTATCCCAGCGA |
| | | CATCGCCGTGGAGTGGGAGAGCAATGGGCAGCCGGAGAAC |
| | | AACTACAAGACCACGCCTCCCGTGCTGGACTCCGACGGCT |
| | | CCTTCTTCCTCTACAGCAAGCTCACCGTGGACAAGAGCAGA |
| | | TGGCAGCAGGGGAACGTCTTCTCATGCTCCGTGATGCATGA |
| | | GGCTCTGCACAACCACTACACGCAGAAGTCTCTCTCCCTGT |
| | | CTCCGGGAAAA |
| VGFB82 | 69 | GAGGTTCAGCTGCTGGAATCTGGCGGAGGATTGGTTCAGC |
| | | CTGGCGGCTCTCTGAGACTGTCTTGCGAGGCCTCTGGCTTC |
| | | GACTTCTCCACCTACGCCATGAACTGGGTCCGACAGGCTCC |
| | | TGGCAAAGGCCTGGAATGGGTCGCCACCATCAATTCCGAG |
| | | GGCACCATCACCTCTCACGCCCCTGCTGTGAAGGGCAGATT |
| | | CACCATCTCTCGGGACAACTCCAAGAACACCGCCTACGTG |
| | | CAGATGAACTCCCTGAGAGCCGAGGACTCCGCCGTGTACT |
| | | ACTGTTCTTCTACCACCGGCACCACACACGGCATGGATGTG |
| | | TGGGGACGCGGCACCACAGTGACCGTTTCTTCTGCCTCCAC |
| | | CAAGGGCCCATCGGTCTTCCCCCTGGCACCCTCCTCCAAGA |
| | | GCACCTCTGGGGGCACAGCGGCCCTGGGCTGCCTGGTCAA |
| | | GGACTACTTCCCCGAACCGGTGACGGTGTCGTGGAACTCA |
| | | GGCGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGCTTGGGCACCCAGACCTACATCTGCAAC |
| | | GTGAATCACAAGCCCAGCAACACCAAGGTGGACAAGAAA |
| | | GTTGAGCCCAAATCTTGTGACAAAACTCACACATGCCCACC |
| | | GTGCCCAGCACCTGAAGCCGCCGGGGGACCGTCAGTCTTC |
| | | CTCTTCCCCCCAAAACCCAAGGACACCCTCATGATCTCCCG |
| | | GACCCCTGAGGTCACATGCGTGGTGGTGAGCGTGAGCCAC |
| | | GAAGACCCTGAGGTCAAGTTCAACTGGTACGTGGACGGCG |
| | | TGGAGGTGCATAATGCCAAGACAAAGCCGCGGGAGGAGC |
| | | AGTACAACAGCACGTACCGTGTGGTCAGCGTCCTCACCGTC |
| | | CTGCACCAGGACTGGCTGAATGGCAAGGAGTACAAGTGCA |
| | | AGGTGTCGAACAAAGCCCTCCCAGCCCCCATCGAGAAAAC |
| | | CATCTCCAAAGCCAAAGGGCAGCCCCGAGAACCACAGGTG |
| | | TACACCCTGCCCCCATCCCGGGAGGAGATGACCAAGAACC |
| | | AGGTCAGCCTGACCTGCCTGGTCAAAGGCTTCTATCCCAGC |
| | | GACATCGCCGTGGAGTGGGAGAGCAATGGGCAGCCGGAG |
| | | AACAACTACAAGACCACGCCTCCCGTGCTGGACTCCGACG |
| | | GCTCCTTCTTCCTCTACAGCAAGCTCACCGTGGACAAGAGC |
| | | AGATGGCAGCAGGGGAACGTCTTCTCATGCTCCGTGATGC |
| | | ATGAGGCTCTGCACAACCACTACACGCAGAAGTCTCTCTCC |
| | | CTGTCTCCGGGAAAA |

TABLE 19
LC nucleotide sequences of selected anti-VEGFR1 antibodies.

| | LC | |
|---|---|---|
| | NUCLEO- | |
| | TIDE SEQ | |
| ANTIBODY | ID NO: | LC NUCLEIC ACID SEQUENCE |
| VGFB54 | 62 | CAGTCTGTGCTGACCCAGCCTCCTTCTGCTTCTGGCACACC |
| | | TGGCCAGAGAGTGACCATCTCCTGTTCCGGCTCTCGGTCCA |
| | | ACATCGGCAACAACTTCGTGAACTGGTATCAGCAGATCCC |
| | | CGGCACCGCTCCTAAGCTGCTGATCTACCTGAACTCCCAGC |
| | | GGCCTTCTGGCGTGCCCGATAGATTCTCCGGCTCTAAGTCT |
| | | GGCACCTCTGCCAGCCTGGCTATCTCTGGCCTGCAGTCTGA |
| | | TGACGAGGCCGACTACTACTGCGCCTCCTGGGACGATTCTC |
| | | TGAACGGCTGGGTTTTCGGCGGAGGCACCAAACTGACAGT |
| | | GCTGGGACAGCCTAAGGCAGCCCCCTCCGTGACCCTGTTCC |
| | | CGCCATCATCCGAAGAACTGCAGGCCAACAAGGCCACGCT |
| | | CGTGTGCCTGATTTCCGACTTCTACCCGGGGGCCGTGACTG |
| | | TGGCCTGGAAGGCAGACTCAAGCCCTGTGAAGGCTGGCGT |
| | | CGAGACTACCACCCCGTCGAAGCAATCCAACAACAAATAC |
| | | GCGGCGTCCAGCTACCTGAGCCTGACCCCTGAGCAGTGGA |
| | | AATCCCACCGGTCCTACTCGTGCCAAGTCACCCACGAGGG |
| | | ATCCACTGTGGAAAAGACCGTGGCGCCGACTGAGTGTTCC |
| VGFB883 | 62 | CAGTCTGTGCTGACCCAGCCTCCTTCTGCTTCTGGCACACC |
| | | TGGCCAGAGAGTGACCATCTCCTGTTCCGGCTCTCGGTCCA |
| | | ACATCGGCAACAACTTCGTGAACTGGTATCAGCAGATCCC |
| | | CGGCACCGCTCCTAAGCTGCTGATCTACCTGAACTCCCAGC |
| | | GGCCTTCTGGCGTGCCCGATAGATTCTCCGGCTCTAAGTCT |
| | | GGCACCTCTGCCAGCCTGGCTATCTCTGGCCTGCAGTCTGA |
| | | TGACGAGGCCGACTACTACTGCGCCTCCTGGGACGATTCTC |
| | | TGAACGGCTGGGTTTTCGGCGGAGGCACCAAACTGACAGT |
| | | GCTGGGACAGCCTAAGGCAGCCCCCTCCGTGACCCTGTTCC |
| | | CGCCATCATCCGAAGAACTGCAGGCCAACAAGGCCACGCT |
| | | CGTGTGCCTGATTTCCGACTTCTACCCGGGGGCCGTGACTG |
| | | TGGCCTGGAAGGCAGACTCAAGCCCTGTGAAGGCTGGCGT |
| | | CGAGACTACCACCCCGTCGAAGCAATCCAACAACAAATAC |
| | | GCGGCGTCCAGCTACCTGAGCCTGACCCCTGAGCAGTGGA |
| | | AATCCCACCGGTCCTACTCGTGCCAAGTCACCCACGAGGG |
| | | ATCCACTGTGGAAAAGACCGTGGCGCCGACTGAGTGTTCC |
| VGFB71 | 65 | CAGTCTGTGCTGACCCAGCCTCCTTCTGCTTCTGGCACACC |
| | | TGGCCAGAGAGTGACCATCTCTTGCTCTGGCGGCTCCTCCA |
| | | ACATCGGCTCCAATTACGTGTCCTGGTATCAGCAGCTGCCC |
| | | GGCACAGCTCCCAAACTGCTGATCTACTTCAACTTCCAGCG |
| | | GCCTTCTGGCGTGCCCGACAGATTCTCTGGCTCTAAGTCTG |
| | | GCACCTCTGCCAGCCTGGCTATCTCTGGACTGCAGAGCGAG |
| | | GACGAGGCCGACTACTATTGTGCCGCCTGGGACGACAGAG |
| | | TGAACGTGTGGGTTTTCGGCGGAGGCACCAAGCTGACAGT |
| | | TCTGGGACAGCCTAAGGCAGCCCCCTCCGTGACCCTGTTCC |
| | | CGCCATCATCCGAAGAACTGCAGGCCAACAAGGCCACGCT |
| | | CGTGTGCCTGATTTCCGACTTCTACCCGGGGGCCGTGACTG |
| | | TGGCCTGGAAGGCAGACTCAAGCCCTGTGAAGGCTGGCGT |
| | | CGAGACTACCACCCCGTCGAAGCAATCCAACAACAAATAC |
| | | GCGGCGTCCAGCTACCTGAGCCTGACCCCTGAGCAGTGGA |
| | | AATCCCACCGGTCCTACTCGTGCCAAGTCACCCACGAGGG |
| | | ATCCACTGTGGAAAAGACCGTGGCGCCGACTGAGTGTTCC |
| VGFB78 | 67 | CAGAGCGTCCTCACTCAGCCTCCCTCAGCTAGTGGCACTCC |
| | | CGGACAGAGGGTAATAATATCTTGCAGCGGTTCAACCAGT |
| | | AATATAGGTAATAATTACGTAAGTTGGTATCAACAGCTCCC |
| | | AGGCACAGCTCCTAAATTGCTCATCTACTATGATCAACGCC |
| | | CTTCCGGTGTTCCTGATAGGTTTTCCGGTTCAAAGAGTGGC |
| | | ACCTCCGCCAGTCTCGCTATATCTGGACTTCAATCAGAGGA |
| | | CGAAGCTGACTATTATTGTGCCGCTTGGGACGACAGCCTGA |
| | | ACGGGTGGGTTTTCGGCGGGGGCACTAAGCTGACAGTCCT |
| | | TGGTCAGCCCAAGGCTGCACCCAGTGTCACTCTGTTCCCGC |
| | | CCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCCGATAGCAGCCCCGTCAAGGCGGGAGTGGA |
| | | GACCACCACACCCTCCAAACAAAGCAACAACAAGTACGCG |
| | | GCCAGCAGCTATCTGAGCCTGACGCCTGAGCAGTGGAAGT |
| | | CCCACAGAAGCTACAGCTGCCAGGTCACGCATGAAGGGAG |
| | | CACCGTGGAGAAGACAGTGGCCCCTACAGAATGTTCA |
| VGFB400 | 67 | CAGAGCGTCCTCACTCAGCCTCCCTCAGCTAGTGGCACTCC |
| | | CGGACAGAGGGTAATAATATCTTGCAGCGGTTCAACCAGT |
| | | AATATAGGTAATAATTACGTAAGTTGGTATCAACAGCTCCC |
| | | AGGCACAGCTCCTAAATTGCTCATCTACTATGATCAACGCC |
| | | CTTCCGGTGTTCCTGATAGGTTTTCCGGTTCAAAGAGTGGC |
| | | ACCTCCGCCAGTCTCGCTATATCTGGACTTCAATCAGAGGA |
| | | CGAAGCTGACTATTATTGTGCCGCTTGGGACGACAGCCTGA |
| | | ACGGGTGGGTTTTCGGCGGGGGCACTAAGCTGACAGTCCT |
| | | TGGTCAGCCCAAGGCTGCACCCAGTGTCACTCTGTTCCCGC |
| | | CCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCCGATAGCAGCCCCGTCAAGGCGGGAGTGGA |
| | | GACCACCACACCCTCCAAACAAAGCAACAACAAGTACGCG |
| | | GCCAGCAGCTATCTGAGCCTGACGCCTGAGCAGTGGAAGT |
| | | CCCACAGAAGCTACAGCTGCCAGGTCACGCATGAAGGGAG |
| | | CACCGTGGAGAAGACAGTGGCCCCTACAGAATGTTCA |
| VGFB82 | 70 | CAGTCTGTGCTGACCCAGCCTCCTTCTGCTTCTGGCACACC |
| | | TGGCCAGAGAGTGACCATCTCTTGCTCCGGCTCCTCCAGCA |
| | | ACATCCAGAACAACTACGTGTACTGGTATCAGCAGCTGCC |
| | | CGGCACCGCTCCTAAACTGCTGATCTACTTCAACTCCCAGC |
| | | GGCCTTCTGGCGTGCCCGATAGATTCTCCGGCTCTAAGTCT |
| | | GGCACCTCTGCCAGCCTGGCTATCTCTGGACTGCAGAGCGA |
| | | GGACGAGGCCGACTACTATTGTGGCACCTGGGACGATTCC |
| | | CTGAACGGCTGGGTTTTCGGCGGAGGCACAAAGCTGACAG |
| | | TGCTGGGTCAGCCCAAGGCTGCACCCAGTGTCACTCTGTTC |
| | | CCGCCCTCCTCTGAGGAGCTTCAAGCCAACAAGGCCACAC |
| | | TGGTGTGTCTCATAAGTGACTTCTACCCGGGAGCCGTGACA |
| | | GTGGCCTGGAAGGCCGATAGCAGCCCCGTCAAGGCGGGAG |
| | | TCGAAACCACCACACCCTCCAAACAAAGCAACAACAAGTA |
| | | CGCGGCCAGCAGCTATCTGAGCCTGACGCCTGAGCAGTGG |
| | | AAGTCCCACAGAAGCTACAGCTGCCAGGTCACGCATGAAG |
| | | GGAGCACCGTGGAGAAGACAGTGGCCCCTACAGAATGTTC |
| | | A |

TABLE 20
Summary of anti-VEGFR1 antibody SEQ ID NOs.

| | VH | VL | | | HC | LC | | |
|---|---|---|---|---|---|---|---|---|
| | Amino | Amino | VH | VL | Amino | Amino | HC | |
| | Acid | Acid | DNA | DNA | Acid | Acid | DNA | LC DNA |
| | SEQ ID | SEQ ID | SEQ ID | SEQ ID | SEQ ID | SEQ ID | SEQ ID | SEQ ID |
| Antibody | NO: | NO: | NO: | NO: | NO: | NO: | NO: | NO: |
| VGFB54 | 31 | 32 | 41 | 42 | 51 | 52 | 61 | 62 |
| VGFB883 | 33 | 32 | 43 | 42 | 53 | 52 | 63 | 62 |
| VGFB71 | 34 | 35 | 44 | 45 | 54 | 55 | 64 | 65 |
| VGFB78 | 36 | 37 | 46 | 47 | 56 | 57 | 66 | 67 |
| VGFB400 | 38 | 37 | 48 | 47 | 58 | 57 | 68 | 67 |
| VGFB82 | 39 | 40 | 49 | 50 | 59 | 60 | 69 | 70 |

### Example 3. Biophysical Characterization and In-Vitro Activity Profiling of Anti-VEGFR1 Antibodies

### Confirmation of Binding to Cells Over-Expressing VEGFR1

Parental CHO-K1 cells, CHO-K1 cells stably expressing human VEGFR1, Ba/F3 parental, and Ba/F3 cells stably expressing murine VEGFR1 were stained for 10 minutes at 4° C. with either no stain, CSFE, CTV, or CTV+CSFE, respectively. Cells were subsequently washed with media containing heat inactivated FBS, collected by centrifugation, combined, resuspended in stain buffer, and plated (95 μL/well; 200,000 of each cell type per well). Antibody samples were added (5 L/well) to a final concentration of 10 nM and incubated for 60 min at 4° C. protected from light. Cells were washed twice with 150 μL/well stain buffer. AlexaFluor647-conjugated anti-human IgG1 secondary antibody was added (100 μL/well) and incubated for 30 minutes at 4° C. protected from light. Cells were washed twice with 150 μL of stain buffer, ultimately resuspended in 30 μL of running buffer and read on an iQue flow cytometer. This single concentration assay was used as a quick confirmation of binding to human and mouse VEGFR1 on cells. A similar assay was performed to confirm binding of candidate antibodies to HEK293 cells stably over-expressing cyno VEGFR1 at a single concentration of 10 nM. To demonstrate specificity to VEGFR1, similar counter-screening assays were also conducted to confirm lack of binding of anti-VEGFR1 antibodies to either HEK293 cells stably expressing human VEGFR2 or K562 cells stably expressing human VEGFR3 at a single high concentration (10 nM) (Table 21).

TABLE 21
Binding to VEGFR1/2/3 on Cells: Cross-reactivity and Specificity.

| | Geomean_{over-expressing}/Geomeanₚₐᵣₑₙₜₐₗ | | |
|---|---|---|---|
| | (Fold Signal/Background) | | |
| | HEK\ | HEK\ | K562\ |
| Antibody | cynoVEGFR1 | huVEGFR2 | huVEGFR3 |
| VGFB82 | 3.30 | 1.34 | 1.01 |
| VGFB54 | 4.40 | 1.52 | 0.97 |
| VGFB78 | 4.46 | 1.75 | 0.99 |
| VGFB71 | 3.80 | 1.27 | 0.98 |
| Goat anti-human VEGFR2 | 1.16 | 27.96 | ND |
| Goat anti-human VEGFR3 | ND | ND | 132.19 |

Threshold for positivity for Cy VEGFR1 was S/B >3
ND = not determined
Goat anti-human VEGFR2 = R&D Systems Catalog #AF357
Goat anti-human VEGFR2 = R&D Systems Catalog #AF349

Full dose-response curves for binding of anti-VEGFR1 antibodies to VEGFR1 on cells were also generated. Parental CHO-K1 cells and CHO-K1 cells stably expressing human VEGFR1 (CHO-K1\hVEGFR1) were cultured in aMEM+10% FBS and aMEM+10% FBS containing 10 μg/mL Puromycin, respectively. CHOK1\hVEGFR1 were stained with CSFE as described above and parental CHO-K1 cells were left unstained. Cells were washed, combined (˜0.5×10⁶ cells/mL), and plated at ˜40,000 cells per well (75 μL/well). Serially diluted antibody samples were then added to the cells and incubated for 1 hour at 37° C. Cells were washed twice with stain buffer and then stained with AF647-conjugated secondary antibody for 30 min at 4° C. Plated cells were washed twice followed by the addition of read buffer containing Sytox Blue (1:1000) and read on an iQue flow cytometer. Data was fit to obtain EC₅₀ values (Table 22).

TABLE 22
Binding of anti-VEGFR1 antibodies
to cells expressing human VEGFR1.

| VEGFR1 mAb | EC50 (M) |
|---|---|
| VGFB80 | 5.967E−10 |
| VGFB54 | 1.008E−10 |
| VGFB71 | 1.095E−10 |
| VGFB82 | 1.363E−10 |
| VGFB78 | 4.732E−10 |

### Ligand Blocking: Biochemical and Cell-Based

The ability of anti-VEGFR1 antibodies to prevent the binding of VEGFA or placental growth factor (PlGF) ligands to VEGFR1 was determined in both a biochemical electro-chemiluminescence (ECL) assay and in a cell-based assay format.

Briefly, MSD MA6000 384 Plates was coated with 0.5 ug/ml of Protein A/G (Thermo Fisher Scientific) or anti-His mAb (R&D) in TBS in cold room for 16 hours. Plates were washed twice with 1×TBS and blocked with 35 ul/well of Blocker A for 1 hour at room temperature. Recombinant human or mouse VEGFR1-Fc proteins (R&D Systems), or His tagged cyno VEGFR1 at 0.5 ug/ml were added to the plates and incubated for 1 hour at room temperature. Unbound VEGFR1 proteins were washed away using 70 ul/well 1×MSD Tris Wash buffer. Unlabeled VEGFR1 ligands or anti-VEGFR1 antibodies in half-log serial dilutions were pre-incubated with bound VEGFR1 for 30 minutes at room temperature and then diluted biotin labeled human VEGFA165 (0.2 nM), mouse VEGFA164 (0.2 nM) or PLGF (2 nM) were added for the binding. Of note, the amino acid sequences of cyno and human VEGFA and PlGF are identical, biotin labeled human VEGF-A and PlGF were used as ligands for cyno VEGFR1 competition assay. The bound ligands were detected by adding 1 ug/ml Sulfo-tagged Streptavidin. The signals were generated and detected on MSD Imager, SECTOR S 600 (Meso Scale C Diagnostics, Serial Number 1201180626620) in presence of 35 ul/well of 2×MSD Read Buffer T. The ECL readings were used to calculate % of binding, IC50 and % of maximal inhibition.

$$\% Binding = ( ( ( Sample - average ( Background ) ) \div ( average ( Total binding ) - average ( Background ) ) ) \times 100$$

Total binding=Biotin ligand only
Background=No blockers and no Biotin ligands
The curves were fitted using Non-linear regression Log (Inhibitor) vs Response-Variable Slope (four parameters).

$$Formula : Y = Bottom + ( Top - Bottom ) / ( 1 + 10 \hat{} ( ( Log IC 50 - X ) * HillSlope ) )$$

The IC50s were extracted from the Prism 8 software.
Maximal % Inhibition=100−Average (% binding at top concentration)

The in vitro efficacy (Maximal % Inhibition) and potency (IC50) of VEGFR1 mAb in displacing VEGFA/PlGF from binding to human, cyno, mouse VEGFR1 are summarized in Table 23 and Table 24. The anti-VEGFR1 antibodies showed potent ligand blocking activity in binding to human and cyno VEGFR1 with ˜100% maximal inhibition. The IC50 values were in sub-nanomolar range, comparable to its ligand. While VGFB80 had no binding or no ligand competition to mouse VEGFR1, other VEGFR1 mAbs showed high blocking efficacy and potency in this assay to mouse VEGFR1.

TABLE 23
VEGFA binding competition to human, cyno and mouse VEGFR1

| | Human VEGFR1 | | Cyno VEGFR1 | | Mouse VEGFR1 | |
|---|---|---|---|---|---|---|
| Unlabeled VEGFA or | Max % | IC50 | Max % | IC50 | Max % | IC50 |
| VEGFR1 mAb | Inh. | (nM) | Inh. | (nM) | Inh. | (nM) |
| VEGFA | 100.0 | 0.156 | 100.0 | 0.261 | 99.9 | 0.150 |
| VGFB80 | 99.2 | 0.291 | 98.6 | 0.104 | 8.5 | >250 |
| VGFB82 | 99.9 | 0.152 | 99.0 | 0.081 | 99.8 | 0.040 |
| VGFB54 | 99.9 | 0.141 | 98.6 | 0.089 | 100.0 | 0.026 |
| VGFB78 | 99.9 | 0.252 | 98.6 | 0.134 | 100.8 | 0.183 |

TABLE 24
PlGF binding competition to human, cyno and mouse VEGFR1

| | Human VEGFR1 | | Cyno VEGFR1 | | Mouse VEGFR1 | |
|---|---|---|---|---|---|---|
| Unlabeled PlGF or | Max % | IC50 | Max % | IC50 | Max % | IC50 |
| VEGFR1 mAb | Inh. | (nM) | Inh. | (nM) | Inh. | (nM) |
| PlGF | 98.6 | 8.923 | 99.2 | 13.34 | 100.0 | 0.163 |
| VGFB80 | 100.1 | 0.348 | 100.2 | 0.105 | 39.1 | >250 |
| VGFB82 | 100.3 | 0.183 | 100.2 | 0.091 | 100.0 | 0.221 |
| VGFB54 | 100.2 | 0.159 | 100.1 | 0.078 | 98.4 | 0.240 |
| VGFB78 | 100.4 | 0.335 | 100.1 | 0.177 | 100.1 | 0.243 |

Ligand blocking on cells was determined as follows: Cell Trace Violet CSFE (ThermoFisher Scientific) stained cells were plated at 15,000 cells per well (30 μL/well; CHO parental or CHO cells engineered to over-express human VEGFR1) in FACS staining buffer. An equivalent volume of serially diluted test antibody was applied to the cells and incubated for one hour at 4° C. Cells were washed twice with 200 μL/well of stain buffer followed by the addition of 100 μL/well of biotinylated VEGF A ligand (500 pM). The cells were incubated at 4° C. for another hour followed by the addition of 50 L/well of AF647-conjugated streptavidin (3 nM). The plate was incubated at 4° C. for 30 min protected from light, washed twice with 200 μL/well of stain buffer, collected by centrifugation, resuspended in running buffer containing Sytox Blue live/dead stain and then read on an Intellicyt iQue Plus flow cytometer. Data was fit to obtain IC₅₀ values. Percent ligand blocking was calculated for each concentration of each test antibody and IC50 values were determined (Table 25).

TABLE 25
VEGF A ligand blocking on cells overexpressing human
VEGFR1 (CHOK1) or overexpressing cyno VEGFR1 (HEK).

| | IC50 (M) HEK/ | IC50 (M) CHOK1/ |
|---|---|---|
| VEGFR1 mAb | cyno VEGFR1 | human VEGFR1 |
| VGFB80 | 5.64E−10 | 4.90E−10 |
| VGFB54 | 8.76E−11 | 7.27E−11 |
| VGFB71 | 9.57E−11 | 7.95E−11 |
| VGFB82 | 2.31E−10 | 1.98E−10 |
| VGFB78 | 8.15E−10 | 6.83E−10 |

### Thermal Stability Determination

Thermal stability of each antibody was determined by NanoDSF method using an automated Prometheus instrument from Nanotemper Technologies. Measurements were made by loading samples at a concentration of 0.25 mg/mL into a 24 well capillary from a 384 well sample plate. Duplicate runs are performed for each sample. Prometheus NanoDSF user interface (Melting Scan tab) is used to set up the experimental parameters for the run. The thermal scans for a typical IgG sample span from 20° C. to 95° C. at a rate of 1.0° C./minute. NIST mAb (and/or CNTO3930, CNTO5825) are included as control and ran in duplicate. The intrinsic fluorescence of the molecule at 330 and 350 nm is used to monitor unfolding during temperature ramp and recorded as changes in fluorescence intensity over time. Tonset is the temperature at which unfolding begins.

TABLE 26
Thermal stability of anti-VEGFR1
antibodies measured by NanoDSF

| | Tₒₙₛₑₜ | T_{agg} | Tm1 | Tm2 |
|---|---|---|---|---|
| Antibody | (° C.) | (° C.) | (° C.) | (° C.) |
| VGFB54 | 63.99 | 72.27 | 71.75 | — |
| VGFB71 | 62.74 | 71.81 | 67.85 | — |
| VGFB78 | 62.57 | 68.48 | 67.86 | — |
| VGFB82 | 61.20 | 73.15 | 71.32 | 77.32 |

Thermal stability of select candidates was also performed by Differential scanning calorimetry (DSC). A MicroCal VP-Capillary DSC instrument was used. Samples were dialyzed overnight into 1×DPBS or another appropriate buffer. Thermal scans were performed by ramping the temperature of the sample and reference cells from 25 to 95° C. at a rate of 60° C./hour. Thermograms were fit using “non-two state” parameters. Tm1 for all samples may represent Fab unfolding, ranges from 68 to 73° C. Tm2 and Tm3 are related to variant IgG1 Fc unfolding.

TABLE 27
Thermal stability of anti-VEGFR1 antibodies measured by DSC

| | | | | ΔH Tm1 | | ΔH Tm2 | | ΔH Tm3 |
|---|---|---|---|---|---|---|---|---|
| Antibody | | Tₒₙₛₑₜ, ° C. | Tm1, ° C. | (kJ/mol) | Tm2, ° C. | (kJ/mol) | Tm3, ° C. | (kJ/mol) |
| VGFB71 | Run 1 | 60 | 68.33 | 1400 | 77.6 | 765 | 83.33 | 365 |
| | Run 2 | 59.7 | 68.5 | 1370 | 77.51 | 720 | 82.38 | 374 |
| VGFB82 | Run 1 | 63.3 | 73.32 | 2620 | 81.62 | 727 | | |
| | Run 2 | 63.3 | 73.32 | 2570 | 81.72 | 709 | | |
| VGFB400 | Run 1 | 59.6 | 69.07 | 1110 | 77.87 | 454 | 83.69 | 218 |
| | Run 2 | 59.6 | 69.24 | 1240 | 77.85 | 704 | 83.8 | 245 |
| VGFB883 | Run 1 | 61.2 | 70.53 | 1600 | 77.77 | 511 | 83.13 | 351 |
| | Run 2 | 61.1 | 70.53 | 1650 | 77.76 | 486 | 82.97 | 370 |

### Affinity Determination by Kinetic Exclusion Assay

Kinetic exclusion assay (KEA; KinExA) was used to analyze the binding of anti-VEGFR1 antibodies to human, mouse and cynomolgus monkeys VEGFR1. KEA is a fluorescence-based solution method, and it is described in detail in the literature (Darling and Brault, 2004). To measure the affinity of an interaction, a series of solutions with a fixed concentration of one interactant (A) and varying concentrations of another interactant (B) are prepared and allowed to reach equilibrium. After equilibration, the concentration of free A is determined by flowing the equilibrated mixture through a column packed with beads modified with reactant B. Free A binds to the beads and is then detected using a fluorescently labeled polyclonal antibody. The sample is loaded into the column at fast flow rates to achieve a short contact time between the mixture and the beads to ensure that the equilibrium is not disrupted (displaced), i.e., and B does not dissociate from the AB complex (kinetic exclusion).

Briefly, fixed concentrations (300 pM, 60 pM, 12 pM, 2.4 or 0.5 pM) of Ab were used to prepare a serial dilution of the antigen (2 nM to 0.52 pM, or 800 pM to 5.37 fM). The complex titrations were incubated at RT to reach binding equilibrium for at least 2 days. After incubation, the samples were run on the KinExA3200 or KinExA4000 instrument to assess free antibody in the reaction. During these studies some of the data were rejected because the experiments failed or failed the acceptance criteria. For mouse VEGFR1 the 60 and 300 pM concentrations were rejected due to high titrant related NSB. The dissociation equilibrium constant, (K_{D}), was determined using the n-curve analysis feature of the KinExA Pro software. This program uses nonlinear least-square regression analysis of the globally fitted data to a 1:1 binding model. (CI=confidence interval).

TABLE 28
Binding affinity of anti-VEGFR1antibodies to
human, mouse and cynomolgus monkey VEGFR1

| | | Affinity (K_{D}) | Affinity (K_{D}) |
|---|---|---|---|
| Antibody | Species | by KinExA (pM) | by SPR (pM) |
| VGFB82 | Human | 0.46 | 10; <6 (n = 2) |
| | | (0.19-0.88; 95% CI) | |
| | Cyno | 0.30 | 38; 23 (n = 2) |
| | | (0.11-0.54; 95% CI) | |
| | Mouse | 23 | Poor fit; |
| | | (15-35; 95% CI) | 10 (n = 2) |
| | Rat | 224 | ~600-1000 |
| | | (6-945; 95% CI) | |
| VGFB883 | Human | ~0.2* | Not determined |
| | | (<0.6-2.1) | |
| | Cyno | ~0.5* | Not determined |
| | | (<0.01-1.4) | |
| | Mouse | >2,000 | Not determined |
| | Rat | Could not be determined due | Not determined |
| | | to very weak affinity | |
| VGFB54 | Human | Not determined | 13, <3.5 |
| | Cyno | Not determined | 16, 18 |
| | Mouse | Not determined | 5300 |
| VGFB78 | Human | Not determined | 20; <6 (n = 2) |
| | Cyno | 0.71 | 87; 17 (n = 2) |
| | | (0.32-1.29; 95% CI) | |
| | Mouse | 3.85 | 4; <6 (n = 2) |
| | | (2.44-5.88; 95% CI) | |
| VGFB1^{a} | Mouse | 0.67 | <1 |
| | | (0.43-0.99; 95% CI) | |
| VGFB80 | Human | 1.26 | 21 |
| | | (0.72-1.93; 95% CI) | |
| | Cyno | 0.48 | 131 |
| | | (0.19-0.85; 95% CI) | |

^{a}Mouse VEGFR1 specific antibody. Does not bind human, cyno, or rat VEGFR1

HDX Epitope Mapping of VEGFR Against 5 mAbs

The epitopes of VEGFR1 against five mAbs, VGFB54, VGFB71, VGFB78, VGFB80, and VGFB82 were mapped by HDX-MS. VGFB80 was a reference molecule not discovered in the antibody generation effort described here. The segments which decreased the free energy more than 2 kcal/mol upon binding to a mAb are HDX-MS defined “strong” epitopes. The segments which decreased the free energy between 1 and 2 kcal/mol upon binding to a mAb are considered “weak” epitopes. The epitopes of VEGFR1 against all tested mAbs are very similar. The strong epitopes of VEGFR1 against VGFB54 are segments 172-177 (FPLDTL, SEQ ID NO: 143), and 201-204 (EIGL, SEQ ID NO: 144). The strong epitopes of VEGFR1 against VGFB71 are segments 172-177 (FPLDTL, SEQ ID NO: 143), 200 (K), and 201-204 (EIGL, SEQ ID NO: 144). The strong epitopes of VEGFR1 against VGFB78 are segments 172-177 (FPLDTL, SEQ ID NO: 143), 200 (K), and 201-204 (EIGL, SEQ ID NO: 144). The strong epitope of VEGFR1 against VGFB82 is segment 201-204 (EIGL, SEQ ID NO: 144). The sequence coverage of VGFW1 was 88% (=285/326) when digested by pepsin/FPXIII mixed bed column after quenched with 0.4 mM FC-14, 8 M urea, 1 M TCEP, pH 3.0. Out of five potential glycosylation sites, N196 was covered by unglycopeptides, and the other four sites were not covered.

TABLE 29
Free energy change upon binding to anti-
VEGFR1 mAb (ΔΔG) (kcal/mol).

| | | | | | | | | |
|---|---|---|---|---|---|---|---|---|
| Starting residue | 141 | 147 | 172 | 178 | 200 | 201 | 211 | 218 |
| End residue | 147 | 153 | 177 | 199 | 200 | 204 | 215 | 228 |
| VGFB71 | −0.3 | −1.2 | −4.2 | 0 | −2.8 | −2.8 | −0.1 | −0.3 |
| VGFB78 | −0.3 | −0.6 | −2.7 | −1.1 | −2.9 | −3.6 | 0.1 | −0.2 |
| VGFB54 | −0.2 | −0.4 | −2.6 | −0.3 | −1.4 | −3.6 | −0.1 | −0.7 |
| VGFB82 | −0.6 | −0.1 | −0.6 | −1 | −0.5 | −3.3 | −0.1 | −0.3 |

HDX Paratope Mapping of 4 mAbs Against VEGFR1

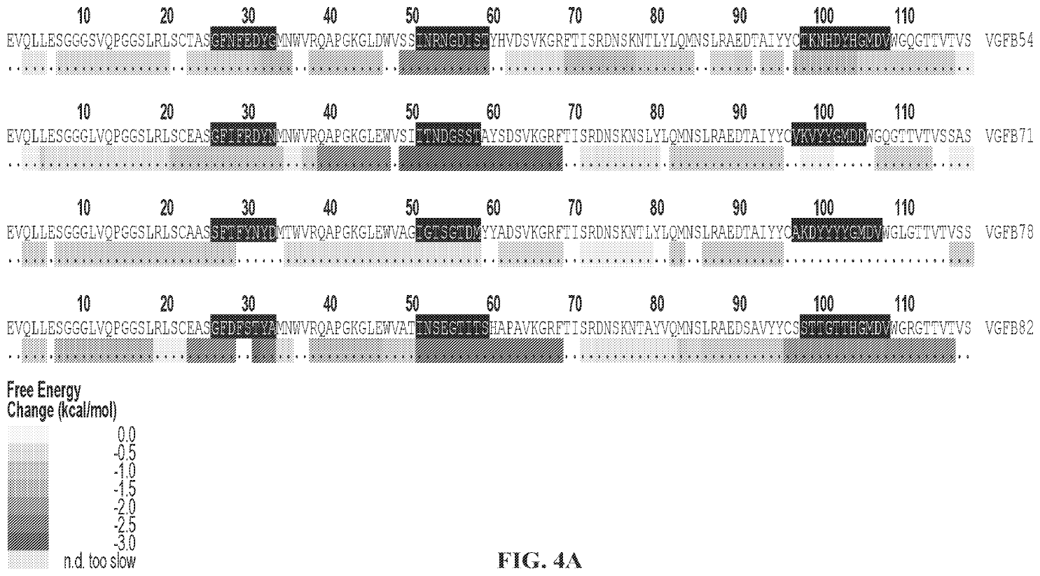

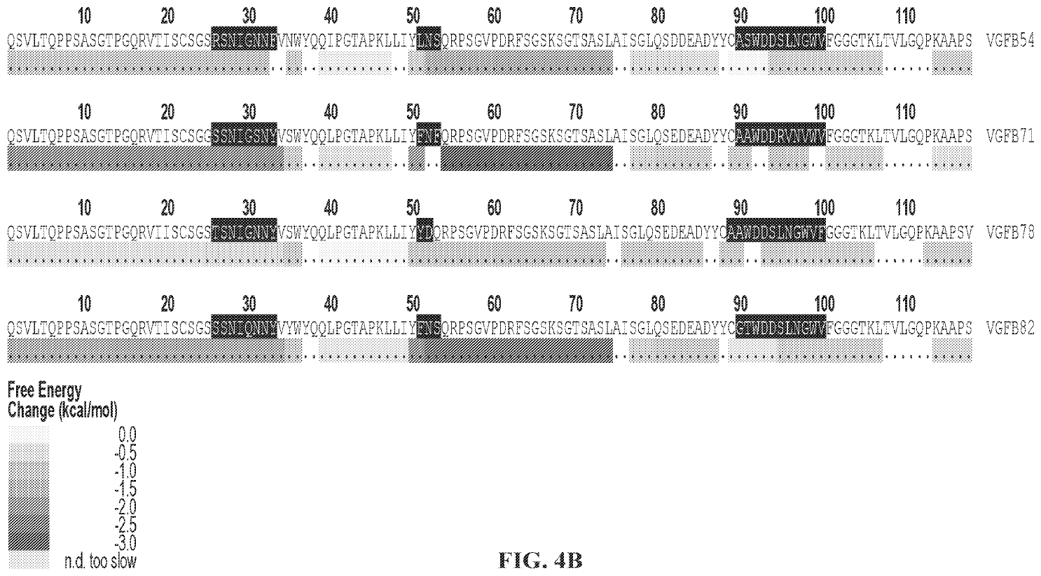

The paratopes of four mAbs against VEGFR1 were mapped by HDX-MS (FIG. 4A and FIG. 4B). The segments which decreased the free energy more than 1 kcal/mol upon binding to a mAb are HDX-MS defined epitopes. The paratopes of VGFB54 against VEGFR1 are heavy chain segments 32-35 (YGMN, HCDR1, SEQ ID NO: 145), 38-46 (RQAPGKGLD, SEQ ID NO: 146), 49-54 (SSINRN, HCDR2, SEQ ID NO: 147), 55-59 (GDIST, HCDR2, SEQ ID NO: 148), 69-77 (FTISRDNSK, SEQ ID NO: 149), and 97-104 (CTKNHDYH, HCDR3, SEQ ID NO: 150) and light chain segments 1-32 (QSVLTQPPSASGTPGQRVTISCSGSRSNIGNN, LCDR1, SEQ ID NO: 151), and 51-74 (LNSQRPSGVPDRFSGSKSGTSASL, LCDR2, SEQ ID NO: 152). The paratopes of VGFB71 against VEGFR1 are heavy chain segments 30-34 (RDYNM, HCDR1, SEQ ID NO: 153), 39-47 (QAPGKGEW, SEQ ID NO: 154), and 49-68 (SIITNDGSSTAYSDSVKGRF, HCDR2, SEQ ID NO: 155) and light chain segments 1-34 (QSVLTQPPSASGTPGQRVTISCSGGSSNIGSNYV, LCDR1, SEQ ID NO: 156), 50-51 (YF, LCDR2), 54-74 (QRPSGVPDRFSGSKSGTSASL, SEQ ID NO: 158) and 94-98 (DRVNV, LCDR3, SEQ ID NO: 159). The paratope of VGFB78 against VEGFR1 is light chain segment 50-73 (YYD-QRPSGVPDRFSGSKSGTSASL, LCDR2, SEQ ID NO: 160). The paratopes of VGFB82 against VEGFR1 are heavy chain segments 7-18 (SGGGLVQPGGSL, SEQ ID NO: 161), 23-28 (EASGFD, HCDR1, SEQ ID NO: 162), 31-33 (TYA, HCDR1), 38-46 (RQAPGKGLE, SEQ ID NO: 163), 51-68 (INSEGTITSHAPAVKGRFT, HCDR2, SEQ ID NO: 164), and 96-116 (CSSTTGTTHGMDVWG, HCDR3, SEQ ID NO: 165) and light chain segments 1-34 (QSVLTQPPSA SGTPGQRVTISCSGSSSNIQNNYV, LCDR1, SEQ ID NO: 166), 50-51 (YF, LCDR2), 52-74 (NSQRPSGVPDRFSGSKSGTSASL, LCDR2, SEQ ID NO: 167) and 95-100 (SLNGWV, LCDR3, SEQ ID NO: 168).

### Tyrosine Kinase Activation Assay

Anti-VEGFR1 antibodies were tested to confirm their inability to activate VEGFR1 signaling. Because of the inherently weak signaling activity of VEGFR1 a previously described cell-based bioassay was employed (Mäkinen, T. et al. EMBO Journal, 20 (17), 4762-4773, 2001). Ba/F3 cells stably expressing a chimeric receptor in which the extracellular domain of human VEGFR1 (residues 1-758; Uniprot Accession #P17948) was fused to the transmembrane and intracellular domains of the mouse erythropoietin receptor (EpoR; (residues 250-507; Uniprot Accession #P14753)) were generated and the IL-3 independent proliferation of these cells was determined in the presence or absence of anti-VEGFR1 antibodies- or hVEGFR1 as a positive control. Briefly, ˜20,000 cells/well were plated in media lacking murine IL-3 (RPMI+10% FBS+0.5 ug/ml puromycin). Murine IL-3 was added to control wells to a final concentration of 10 ng/mL. Other wells received either an anti-VEGFR1 antibody (final concentration 10 or 50 nM), hVEGFA165 (5 nM; R&D Systems), or basal media (without mIL-3). Cells were incubated at 37° C., 5% CO₂ for 72 hours. Proliferation was measured by addition of 50 μL/well CellTiter Glo (Promega), incubation for 10 minutes at room temperature. Finally, the plate was read on an Envision 2105 plate reader using Ultrasensitive Luminescence protocol. Proliferation of the BaF3\hVEGFR1-mEpoR cells was compared to mIL-3-dependent conditions. Unlike hVEGFA (5 nM), which was demonstrated to rescue viability of the cells in the absence of mIL-3, none of the anti-VEGFR1 antibodies tested were able to promote IL-3 independent growth at any of the concentrations tested (Table 30). This result is consistent with the inability of the anti-VEGFR1 antibodies to stimulate tyrosine kinase activation or further downstream signaling.

TABLE 30
Tyrosine kinase activation by anti-VEGFR1 antibodies

| | % | |
|---|---|---|
| | viability | Comment |
| media + IL3 | 100.00 | IL-3 dependent growth |
| media (no IL3) | 0.15 | |
| hVEGFA 5 nM | 111.50 | Viability rescued by ligand-mediated |
| | | chimeric VEGFR1-EpoR dimerization |
| VGFB82 (10 nM) | 0.48 | Does not stimulate TK activity of |
| | | chimeric VEGFR1-EpoR |
| VGFB82 (50 nM) | 0.36 | Does not stimulate TK activity of |
| | | chimeric VEGFR1-EpoR |
| VGFB883 (10 nM) | 0.28 | Does not stimulate TK activity of |
| | | chimeric VEGFR1-EpoR |
| VGFB883 (50 nM) | 0.33 | Does not stimulate TK activity of |
| | | chimeric VEGFR1-EpoR |

### Specificity Cell Surface-Ome

One risk associated with broad species cross-reactive antibodies is an increased off-target liability. Cell microarray technology was performed (Retrogenix Ltd.) to confirm the specificity of anti-VEGFR1 antibodies for VEGFR1 and the absence of any “off-target” liabilities.

This human cell microarray methodology has been previously described in the literature (Freeth, J. et al, SLAS Discovery, 25 (2), 223-230, 2020). Primary screening was performed on slides arrayed with fixed HEK293 cells with each spot of HEK293 cells expressing either a unique protein from the cell surface-ome, a cell surface tethered human secreted protein, or a heterodimeric human cell surface receptor. In total, 5,528 unique gene products were represented in this primary screen (4,103 human plasma membrane proteins (+some untethered secreted proteins)+1,425 cell surface tethered human secreted proteins). Briefly, slides containing fixed transfected HEK293 cells were overlaid with a solution of test antibody (2 μg/mL), incubated, and washed. Binding was assessed using AlexaFluor647-conjugated anti-human IgG Fc detection antibody and fluorescence quantitation on ImageQuant. As expected, all anti-VEGFR1 molecules tested exhibited binding to isoforms of human VEGFR1 (FLT1 isoform 1, 2 and 3) (Table 31-34). VGFB54, VGFB80, and VGFB82 showed no binding to any other surface-ome proteins captured by this screen. Two of the anti-VEGFR1 antibodies (VGFB71 and VGFB78) exhibited unexpected binding to unrelated cell surface proteins (Table 32) Nectin1, FGFR2 (Fibroblast growth factor receptor 2), ISLR (Immunoglobulin superfamily containing leucine-rich repeat protein) and CRELD2 (Protein disulfide isomerase CRELD2). Located in the ER, off-target binding to CRELD2 was of less concern. Nectin-1, FGFR2, and ISLR, on the other hand, display broad tissue expression profiles. Cross-reactivity to these proteins is undesirable because binding to these targets might have a significant impact on pharmacokinetic and biodistribution profiles of the molecules. This off-target binding was subsequently confirmed by flow cytometry on live HEK293 cells transiently expressing these ‘off-target’ proteins identified in the primary screen. These data were used to further select anti-VEGFR1 antibodies with no known off-target liabilities.

TABLE 31
Retrogenix specificity data. Summary of VEGFR1 specific
membrane hits (desired hits) above very weak intensity.

| | | | VEGFR1 (desired reactivity) | | | |
|---|---|---|---|---|---|---|
| | specific | | | | | KDR + FLT1 |
| | membrane | Gene ID | FLT1 | FLT1 | FLT1 | NM_002253.2 + |
| | hist above | Accession | NM_001160030.1 | NM_002019.4 | NM_001159920.1 | NM_002019.4 |
| | very weak | Topology | TS | PM | S | HD |
| Antibody | intensity | isoform | 3 | 1 | 2 | 1 + 1 |
| VGFB54 | 3 | | medium | weak | weak | very weak |
| VGFB71 | 8 | | medium | weak | weak | Very weak |
| VGFB78 | 4 | | medium | weak | very weak | very weak |
| VGFB80 | 4 | | medium | weak | weak | Weak |
| VGFB82 | 4 | | medium | weak | weak | Weak |

TABLE 32
Retrogenix specificity data. Summary of non-VEGFR1 specific
membrane hits (undesired hits) above very weak intensity.

| | | Non-VEGFR1 (undesired reactivity) | | | | |
|---|---|---|---|---|---|---|
| | | NECTIN1 | NECTIN1 | NECTIN1 | NECTIN1 | |
| | Gene ID | NM_002855.4 | NM_203285.1 | NM_203286.1 | NM_203286.1 | FGFR2 |
| | Accession | PM | PM | TS | S | NM_022970.3 |
| | Topology | Isoform | Isoform | Isoform | Isoform | PM |
| Antibody | isoform | Delta | Alpha | Gamma | Gamma | Isoform 3 |
| VGFB54 | | | | | | |
| VGFB71 | | strong | strong | strong | weak/ | weak |
| | | | | | med | |
| VGFB78 | | | | | | |
| VGFB80 | | | | | | |
| VGFB82 | | | | | | |

| | | Non-VEGFR1 (undesired reactivity) | | | |
|---|---|---|---|---|---|
| | | ISLR | | | |
| | Gene ID | BC022478 | CRELD2 | | |
| | Accession | S | NM_024324.4 | SLCO1B3 | TMED1 |
| | Topology | Single | TS | BC141525 | BC002443 |
| Antibody | isoform | form | Isoform 1 | PM | PM |
| VGFB54 | | | | | |
| VGFB71 | | | | | |
| VGFB78 | | weak | weak | v.weak | |
| VGFB80 | | | | | v.weak |
| VGFB82 | | | | | |

TABLE 33
Retrogenix specificity data. Summary of common Fc gamma receptor
specific membrane hits (expected hits) above very weak intensity.

| | Common Fc gamma receptor (expected reactivity) | | | | |
|---|---|---|---|---|---|
| Gene ID | IGHG3 | FCGRIA | FCGR2A | FCGR2A | FCGR3A |
| Accession | BC089421 | BC032634 | DQ892037 | NM_001136219.1 | BC017865.1 |
| Topology | M | PM | PM | PM | PM |

| | | | | | |
|---|---|---|---|---|---|
| VGFB54 | strong | weak | | | |
| VGFB71 | strong | weak | | | |
| VGFB78 | strong | weak | | | |
| VGFB80 | strong | med/strong | weak/med | weak | weak |
| VGFB82 | strong | weak | | | |

TABLE 34
Retrogenix specificity data. Summary of common Fc gamma receptor
specific membrane hits (expected hits) above very weak intensity.

| | | Common Fc gamma receptor (expected reactivity) | | | | |
|---|---|---|---|---|---|---|
| | | FCGR3A + CD247 | FCGR3A + FCER1G | | | |
| | Gene ID | BC017865.1 + | BC017865.1 + | CXCL12 | IGF2 | IGF1 |
| | Accession | NM_198053.2 | NM_004106.1 | NM_000609.6 | NM_001291862.2 | NM_001111285.2 |
| Antibody | Topology | HD | HD | TS | TS | TS |

| | | | | | |
|---|---|---|---|---|---|
| VGFB54 | | | v.weak | v.weak | v.weak |
| VGFB71 | | | v.weak | v.weak | v.weak |
| VGFB78 | | | v.weak | v.weak | Weak |
| VGFB80 | weak | weak/med | v.weak | v.weak | Weak |
| VGFB82 | | | v.weak | v.weak | v.weak |

### Example 4. Systemic Pharmacokinetics and Target Engagement Biomarker Profile of Anti-VEGFR1 Antibodies in Cynomolgus Monkeys

In addition to biophysical and in vitro activity profiling of the anti-VEGFR1 antibodies, the systemic pharmacokinetics (PK) and target engagement (TE) biomarker profiles were characterized in cynomolgus monkeys.

Naïve male cynomolgus monkeys (Macaca Fascicularis, Age 2 to 7 years, Envigo RMS) were randomized into groups of 2 animals each by body weight. VGFB54, VGFB78, VGFB82 and VGFB80 at 0.3, 2, and 10 mg/kg were dosed intravenously (IV) given over ˜ 2 minutes on day 1 and a subsequent subcutaneous (SC) dose on day 15. Blood samples (total 0.5-2 mL) were collected via a femoral vein into tubes containing K2 EDTA from each animal at pre-dose, and the time points as follows 1, 6, 24, 48, 96, and 168 hours post IV dose, and 1, 6, 24, 48, 96, 168, and 336 hours post SC dose. Blood samples were maintained at 2-8° C. prior to centrifugation to obtain plasma (within 1 hour of collection). Plasma samples were harvested into PK and TE aliquots, and stored at −70° C. until sample analysis.

### Immunoassay Bioanalysis of Anti-VEGFR1 Antibodies

The “Total” and “Free” mAb concentration of anti-VEGFR1 mAbs in cynomolgus monkey plasma was determined using an immunoassay method that employed a typical sandwich format with electro-chemiluminescent detection. Briefly, MSD Gold streptavidin plate (Meso Scale Discovery) was prewet with blocking buffer and tapped dry. A master mix solution containing final working concentrations of capture and detection reagent was added to the plate (35 μL/well) along with diluted standards, quality controls (QCs) and samples (15 μL/well) and incubated for 60 minutes. After wash, 1×MSD-T read buffer was added to all wells. The plate was immediately read on the MSD Sector S600 imager. The quantifiable range for “Total” and “Free” VEGFR1 mAb was defined as 0.010-2560 ug/mL and 0.020-5120 ug/mL, respectively, with a 10× minimum required dilution for plasma samples. For the measurement of “total” mAb, biotinylated and ruthenium-labeled anti-human Fc mAb (R10Z8E9) was used as the capture/detection reagent. For the measurement of “free” mAb, biotinylated recombinant cynomolgus monkeys VEGFR1 [D1-D3]-Avi6×His, and ruthenium-labeled anti-human Fc mAb (R10Z8E9) were used as the capture/detection reagents.

The MSD output files containing the raw ECL counts were swept into Cyberlab Content Manager (Agilent) and imported into Watson LIMS (Thermo Scientific) regression software for analysis. The Watson study regression was predefined during assay qualification and used a 5-parameter logistic (Auto-estimate) fit with 1/Y2 weighting factor.

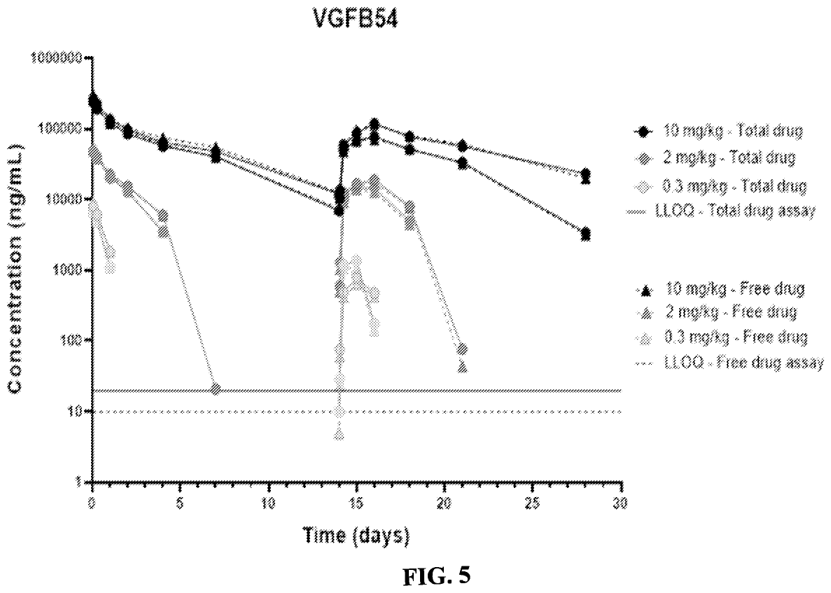

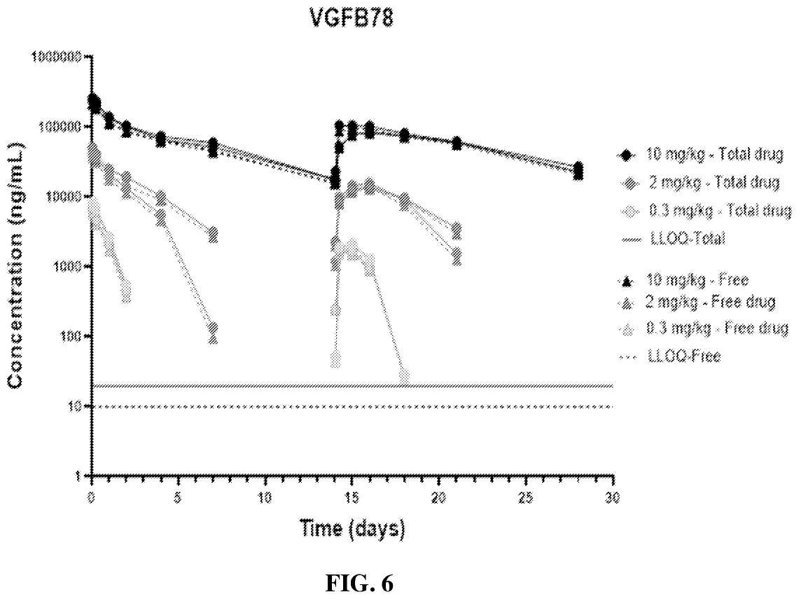

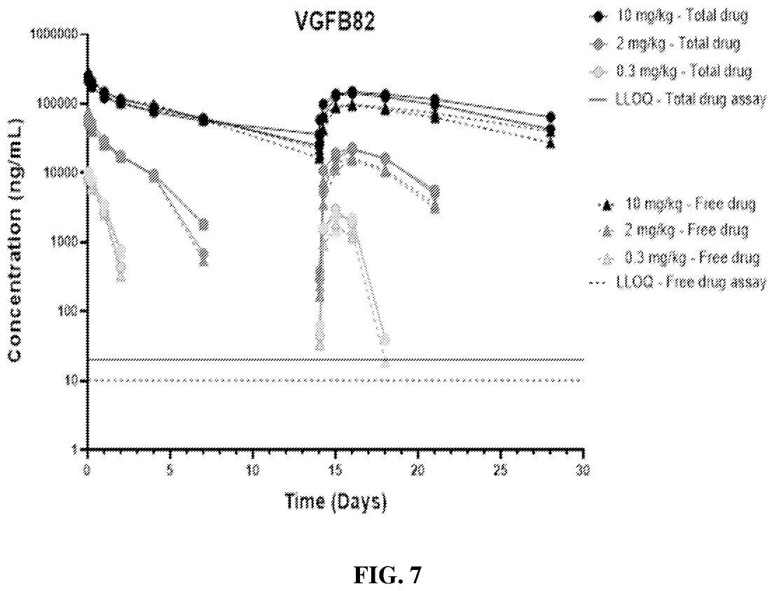

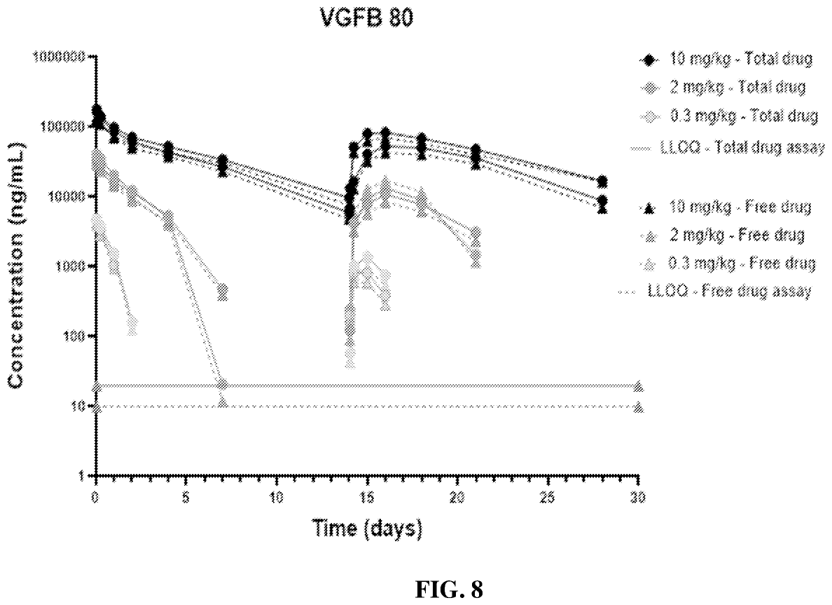

The plasma drug concentration-time profiles are illustrated in FIG. 5-8. Total and free concentrations were similar across all time points for all four molecules. Plasma PK parameters, which were estimated using non-compartmental analysis, are shown in Table 35. Terminal half-life (T1/2), maximum plasma drug concentration (Cmax), and area under the drug serum concentration-time curve (AUC) of one dose interval, increased with dose in a greater than dose-proportional manner. The nonlinear PK profiles, and the shorter half-lives at lower doses are suggestive of target-mediated drug disposition (TMDD). Mean half-life at 10 mg/kg IV for VGFB54, VGFB82, and VGFB80 was ≈4.9, 5.7, 4.5 days, respectively (based on non-compartment analysis of total PK). However, due to significant TMDD, short sampling time and that the terminal phase may not be fully characterized, T1/2, and clearance (CL) should be interpreted with caution. The monkey PK profile of VGFB82, VGFB54, and VGFB80 were also characterized using a minimal physiologically based PK model. The SC bioavailability for VGFB54, VGFB82, and VGFB80 was ˜83.7%, 95.4% and 87.7%.

TABLE 35
Mean pharmacokinetic parameters of VEGFR1 mAb VGFB82,
VGFB54, VGFB78, and VGFB80 following a single IV dose
on day 0 and subsequent SC dose on Day 14 at 0.2, 3,
or 10 mg/kg administration in cynomolgus monkeys.

| | | | | | |
|---|---|---|---|---|---|
| | | AUCₗₐₛₜ | AUC_{inf} | CL | |
| Dose mg/kg | Cₘₐₓ | (μg · | (μg · | (mL/ | T_{1/2} |
| (Route) | (μg/mL) | day/mL) | day/mL) | day/kg) | (day) |

VGFB82

| | | | | | | |
|---|---|---|---|---|---|---|
| 0.3 | (IV) | 9.34 | 7.38 | 7.83 | 38.85 | 0.47 |
| 2 | (IV) | 54.32 | 102.14 | 104.92 | 19.06 | 1.28 |
| 10 | (IV) | 234.93 | 699.25 | 1177.42 | 8.51 | 5.68 |
| 0.3 | (SC) | 2.68 | 4.62 | 5.07 | NA | 0.48 |
| 2 | (SC) | 22.16 | 97.86 | 117.11 | NA | 2.36 |
| 10 | (SC) | 146.58 | 1616.39 | 2239.42 | NA | 7.7 |

VGFB54

| | | | | | | |
|---|---|---|---|---|---|---|
| 0.3 | (IV) | 7.42 | 3.98 | 4.87 | 63.37 | 0.4 |
| 2 | (IV) | 48.24 | 69.64 | 72.7 | 27.56 | 0.84 |
| 10 | (IV) | 252.81 | 598.77 | 905.44 | 11.12 | 4.91 |
| 0.3 | (SC) | 1.08 | 1.41 | 1.86 | NA | 0.61 |
| 2 | (SC) | 16.9 | 51.85 | 58.73 | NA | 1.19 |
| 10 | (SC) | 98.85 | 713.23 | 815.91 | NA | 4.08 |

VGFB78

| | | | | | | |
|---|---|---|---|---|---|---|
| 0.3 | (IV) | 7.77 | 5.8 | 6.19 | 49.35 | 0.5 |
| 2 | (IV) | 46.47 | 87.88 | 92.39 | 22.93 | 1.31 |
| 10 | (IV) | 250.69 | 674.71 | 1141.72 | 8.81 | 5.91 |
| 0.3 | (SC) | 1.96 | 3.44 | 4.88 | NA | 1.19 |
| 2 | (SC) | 14.93 | 64.07 | 72.79 | NA | 1.99 |
| 10 | (SC) | 95.17 | 961.98 | 1183.48 | NA | 5.97 |

VGFB80

| | | | | | | |
|---|---|---|---|---|---|---|
| 0.3 | (IV) | 4.71 | 3.06 | 3.46 | 87 | 0.42 |
| 2 | (IV) | 37.62 | 63.81 | 64.24 | 31.15 | 0.79 |
| 10 | (IV) | 174.8 | 435.62 | 634.35 | 15.93 | 4.47 |
| 0.3 | (SC) | 1.11 | 1.69 | 2.28 | NA | 1.57 |
| 2 | (SC) | 12.21 | 50.38 | 58.59 | NA | 2.11 |
| 10 | (SC) | 68.77 | 614.63 | 702.6 | NA | 4.41 |

Cₘₐₓ, maximum (peak) concentration;
AUCₗₐₛₜ, area under the concentration-time curve up to the last sampling point;
AUC_{inf}, area under the concentration-time curve extrapolated to infinity;
CL, total clearance;
T_{1/2}, half-life;
NA, not available

### Plasma PlGF in Response to Administration of VEGFR1 mAb

In addition to VEGFA, VEGFR1 also binds to placental growth factor (PlGF). Unlike VEGFA that binds to both VEGFR1 and VEGFR2, PlGF is a selective ligand of VEGFR1. As PlGF and VEGFA share overlapped binding domains on VEGFR1 (D2-D3) with comparable binding affinity, anti-VEGFR1 antibody that blocks VEGFA binding will competitively block the binding of PlGF to VEGFR1, resulting in an increase in the level of PlGF.

Total plasma PlGF levels including both the unbound and bound to VEGFR1 in cynomolgus monkey were determined using a stepwise electro-chemiluminescence assay (Human PlGF V plex Kit, MSD K151MED-4). The capture antibody coated plate was blocked and incubated for 1 hr. The PlGF calibrators (human PlGF, R&D Systems), quality controls (QCs), and plasma samples were diluted 1:10 with buffer containing anti-VEGFR1 antibody (167 μg/mL), which binds to soluble VEGFR1 hence relieving the signal suppression caused by VEGFR1 binding to PlGF. Following the incubation at room temperature for at least 15 min, the mix was added to the washed MSD plate after blocking and incubated at 4° C. overnight. After washing, detection antibody conjugated with SulfoTag was added and incubated at room temperature for 2 hrs. The MSD plate was washed and 1×MSD-T read buffer was added to all wells. The plate was immediately read on the MSD Sector S600 imager.

The MSD output files containing the raw ECL counts were imported into Watson LIMS (Thermo Scientific) regression software for analysis. The Watson study regression was predefined during assay qualification and used a 4-parameter logistic fit with 1/Y2 weighting factor.

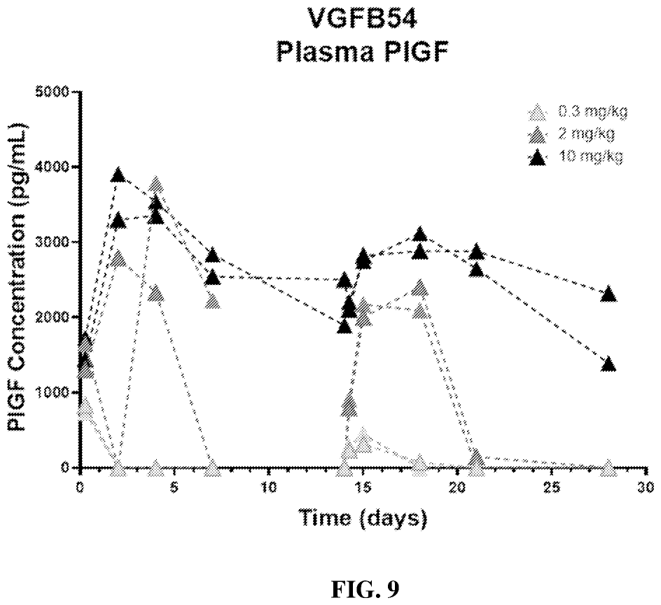

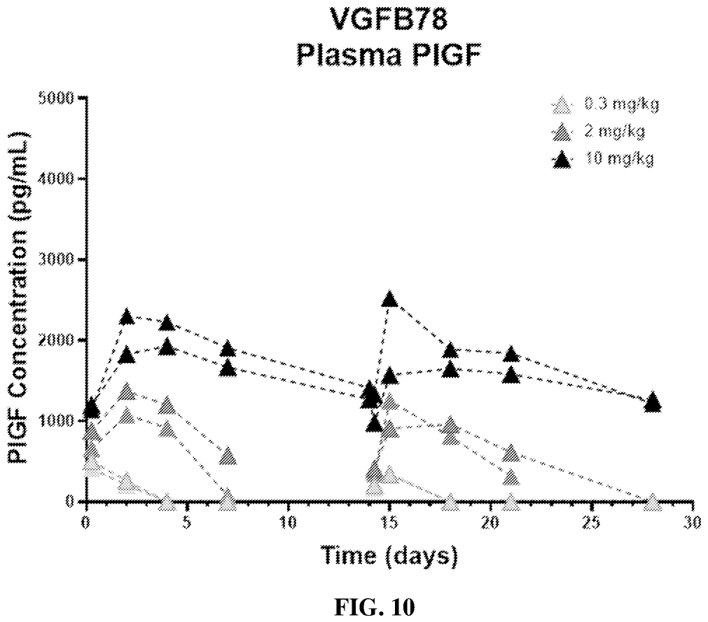

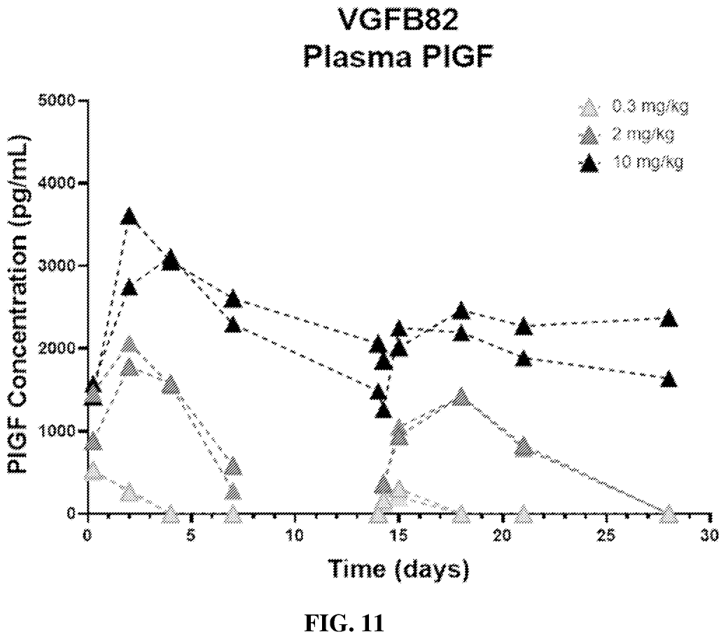

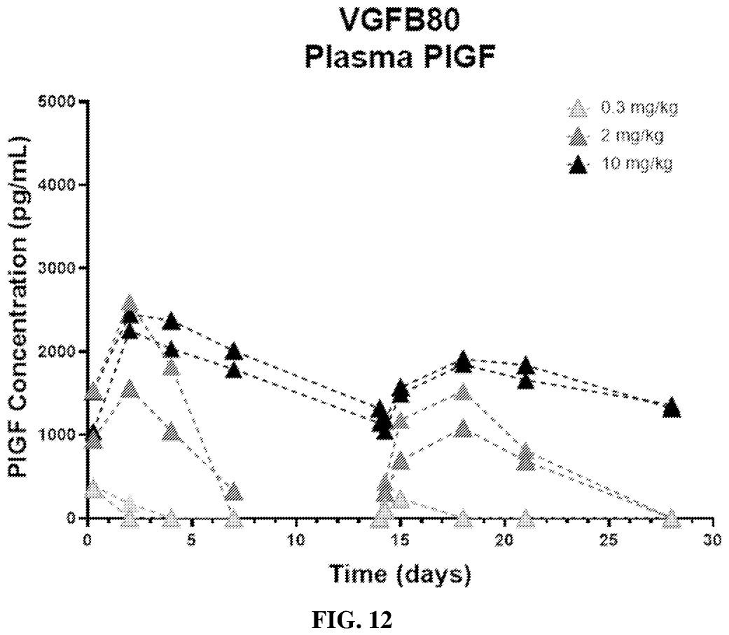

Following IV or SC administration of VEGFR1 mAb, systemic PlGF level was increased in a dose- and PK-related manner (FIG. 9-12). The increase in PlGF level is comparable between VGFB82 and VGFB54, which is trending higher than VGFB80 and VGFB78.

### Example 5. Target Engagement Biomarker Profile of Mouse Reformatted Anti-VEGFR1 Leads in db/db Mice

To reduce the immunogenicity of human anti-VEGFR1 mAb in mice enabling chronic dosing and the efficacy study in mice, mouse reformatted version of human anti-VEGFR1 antibodies were generated, in which the variable regions of human anti-VEGFR1 mAb (VH/VL) were grafted onto mouse constant regions that are made up of silent murine IgG1_D265A (CH1, CH2, CH3) for the heavy chain and the constant region of murine lambda for the light chain. The control is a control antibody of the same isotype that does not bind VEGFR1.

For the measurement of mouse PlGF, mouse PlGF-2 Quantikine ELISA Kit (R&D System Catalog MP200) was used. Test plasma samples were diluted with Calibrator Diluent RD 5-17 from 1:4 to 1:15 according to the dose levels and time points to keep readouts within the linear detection range of the assay (assay range from 23.4-1500 μg/mL with the sensitivity at 1.84 pg/mL). The plates were read on a SpectraMax Plate Reader set to 450 nm (Molecular Devices Model Paradigm), and the concentrations of PLGF-2 in mouse plasma samples were calculated using SoftMax® Pro 7.1.

Upon repeated subcutaneous weekly administration in db/db female mice (The Jackson laboratory, Stock No. 000642) on day 0, 7 and 14 at 1 mg/kg or 10 mg/kg, mouse reformatted VGFB54, VGFB78 and VGFB82 elicited different level of PlGF response at the same dose level (Table 36 and 37). Reformatted VGFB78 induced the most robust increase of plasma PlGF among 3 leads. Reformatted VGFB82 also led to an increase of PLGF. However, compared to VGFB78, VGFB82 induced PlGF response at a significantly lower level in mice (˜4-fold lower than VGFB78 at 10 mg/kg). Reformatted VGFB54 had a minimal increase in TE biomarkers in mice.

TABLE 36
PlGF plasma concentration (pg/mL, mean ± SD) over time
following three SC administrations of mouse reformatted VGFB54
and VGFB78 on day 0, 7 and 14 in db/db mice (N = 3/timepoint)

| | Timepoint | | | | | |
|---|---|---|---|---|---|---|
| | (plasma sample | | Mouse reformatted VGFB54 | | Mouse reformatted VGFB78 | |
| Timepoint | collection) | | 1 mg/kg | 10 mg/kg | 1 mg/kg | 10 mg/kg |
| (SC, QW) | (N = 3) | | (N = 6) | (N = 6) | (N = 6) | (N = 6) |
| Day 0 | 6 | hr | 73.79 ± 25.59 | 78.12 ± 5.92 | 113.24 ± 30.73 | 928.93 ± 329.34 |
| Day 0 | 24 | hr | 56.52 ± 2.70 | 182.44 ± 7.28 | 352.23 ± 88.20 | 1798.69 ± 133.21 |
| (1st dose) | 72 | hr | 67.25 ± 4.83 | 110.37 ± 9.06 | 223.94 ± 89.24 | 3307.23 ± 410.88 |
| Day 7 | Pre 3rd dose | | 58.06 ± 11.66 | 215.23 ± 10.86 | 155.70 ± 10.11 | 2364.03 ± 261.46 |
| (2nd dose) | | | | | | |
| Day 14 | 6 | hr | 70.65 ± 5.96 | 127.08 ± 9.70 | 118.09 ± 4.92 | 3292.55 ± 634.13 |
| (3rd dose) | 24 | hr | 86.37 ± 11.74 | 243.72 ± 11.16 | 493.73 ± 31.47 | 3822.23 ± 622.39 |
| | 72 | hr | 74.01 ± 5.44 | 133.67 ± 15.67 | 209.48 ± 29.94 | 3785.32 ± 426.27 |
| | 7 | day | 99.73 ± 40.78 | 223.14 ± 7.82 | 159.10 ± 8.85 | 2340.42 ± 310.12 |
| | 14 | day | 74.07 ± 2.99 | 105.37 ± 14.89 | 74.57 ± 6.34 | 1111.91 ± 342.43 |

TABLE 37
PlGF plasma concentration (pg/mL, mean ± SD) over time
following three SC administrations of mouse reformatted
VGFB82 on day 0, 7 and 14 in db/db mice (N = 3/timepoint)

| | Timepoint | | | | |
|---|---|---|---|---|---|
| | (plasma sample | | Mouse reformatted VGFB82 | | Control |
| Timepoint | collection) | | 1 mg/kg | 10 mg/kg | 10 mg/kg |
| (SC, QW) | (N = 3) | | (N = 6) | (N = 6) | (N = 6) |
| Day 0 | 6 | hr | 117.53 ± 19.98 | 237.48 ± 15.87 | 88.98 ± 9.35 |
| (1st dose) | 24 | hr | 288.62 ± 58.80 | 534.29 ± 40.46 | 114.24 ± 9.95 |
| | 72 | hr | 193.42 ± 12.80 | 790.23 ± 145.03 | 100.84 ± 5.24 |
| Day 7 | Pre 3rd dose | | 261.3 ± 38.59 | 744.90 ± 71.63 | 79.84 ± 2.81 |
| (2nd dose) | | | | | |
| Day 14 | 6 | hr | 152.13 ± 13.56 | 959.72 ± 3.54 | 116.02 ± 10.45 |
| (3rd dose) | 24 | hr | 303.77 ± 26.49 | 920.58 ± 71.13 | 97.87 ± 17.32 |
| | 72 | hr | 190.50 ± 20.47 | 1001.51 ± 89.95 | 162.05 ± 83.63 |
| | 7 | day | 273.14 ± 14.27 | 614.01 ± 77.96 | 99.00 ± 7.57 |
| | 14 | day | 109.11 ± 62.26 | 1313.81 ± 724.80 | 137.78 ± 4.73 |

### Example 6. Effect of VGFB82 and VGFB78 in Hypertensive Uni-Nephrectomized db/db Mice on Albuminuria Reduction

A severe progressive diabetic kidney disease murine model named herein after ReninAAV uNx db/db model was used to evaluate the efficacy of anti-VEGFR1 lead mAbs (Harlan S M, et al. J Am Soc Nephrol 2018; 29:477-91; Harlan S M, et al. Am J Physiol Regul Integr Comp Physiol 2015; 309: R467-74). In this model, persistent hypertension induced by the delivery of Renin expressing adeno-associated virus further accelerates the disease progression and kidney injury in uni-nephrectomized db/db mice. ReninAAV uNx db/db mice show severe renal dysfunction characterized by significant elevations in urinary albumin-to-creatinine ratio (UACR), reduced glomerular filtration rate (GFR) and increased serum creatinine. Histopathological characterization showed resembling features of advanced human diabetic kidney disease, including mesangial expansion, glomerular sclerosis, tubular degeneration as well as tubulointerstitial inflammation and fibrosis.

Female db/db mice were uni-nephrectomized at 7-8 weeks of age (Surg 3041 from the Jackson Laboratory), and at 12-13 weeks infected with a renin expressing adeno-associated virus (1×10¹⁰ genome copies). Six weeks later, mice were randomized into groups of 9-15 animals each, based on their UACR, systolic blood pressure, and body weight using the weightage of 0.8, 0.1, 0.1 respectively on IRINI software. A blood glucose >300 mg/dL and UACR of >5000 mg/g were used as inclusion criteria to enter randomization. Mice received either vehicle, Lisinopril (10 mg/kg in drinking water), isotype control, low- or high-doses of mouse reformatted VGFB82 or VGFB78 by subcutaneous injection at specified dosing frequency for 4-6 weeks. Urine samples were collected by housing mice individually in metabolic cages (MMC100, Hatteras Instruments) for 2 h prior to the treatment, and biweekly post dose. Urinary concentrations of albumin and creatinine were measured using Sekisui Diagnostics Microalbumin assay kit (Cat #252-20), Enzymatic Creatinine assay kit (Cat #265-30) and Vet Axcel Chemistry Analyzer (Alfa Wassermann). Plasma samples were collected for PlGF measurement.

Data were presented as mean+/−SE. GraphPad Prism (Ver 8.0 GraphPad) and a two-way repeated measurement ANOVA or/and a linear Mixed-effect model was used for treatment comparisons.

Mouse reformatted VGFB82 (VGFB877) at 3 and 30 mg/kg were dosed SC weekly (QW) for 4 weeks, followed by 30 mg/kg three times weekly (TW) and 90 mg/kg weekly, respectively, for another 2 weeks. Table 38 showed UACR and the percentage change from the baseline at week 2, 4 and 6 post doses. Table 39 showed plasma PlGF level at week 4 and 6 post doses. Vehicle-treated group displayed a continued and sustained UACR increase throughout the treatment. Lisinopril, an angiotensin-converting enzyme inhibitor (ACEi, standard of care), significantly decreased the UACR to −26% from its baseline in comparison to vehicle control at week 4. Mice treated with VGFB877 at 3 mg/kg QW for 4 weeks did not show discernable changes in UACR when compared to the vehicle group. The high dose of VGFB877 at 30 mg/kg/QW partially halted the UACR progression when compared to vehicle-treated mice at week 4 (% change from baseline+15% for VGFB877 vs+83% for vehicle). When the dose and dosing frequency increased from 3 mg/kg QW to 30 mg/kg TW, UACR % change from baseline was decreased from +63% to +28% at week 6. When the dose adjusted from 30 mg/kg QW to 90 mg/kg QW, UACR % change from baseline was decreased from +15% to +6% at week 6. Consistent with improved UACR reduction, the plasma PlGF level waste elevated when the dose level or frequency of VGFB82 increased.

TABLE 38
Effect of reformatted VGFB82 (VGFB877) in ReninAAV
uNx db/db mice on UACR and % change from

| | Treatment | Vehicle | | | ACEi 10 mg/kg | | |
|---|---|---|---|---|---|---|---|
| | Week | Mean | SEM | N | Mean | SEM | N |
| UACR | 0 | 10002.8 | 845.1 | 12 | 9929.1 | 1117.1 | 9 |
| (μg/mg) | 2 | 17933.1 | 2415.7 | 11 | 8390.1 | 1314.2 | 9 |
| | 4 | 15517.9 | 2344.2 | 8 | 7098.9 | 1168.7 | 9 |
| | 6 | 15745.9 | 3384.6 | 7 | 5643.6 | 960.4 | 9 |
| % Change | 2 | 76.83 | 14.63 | 11 | −15.24 | 8.38 | 9 |
| from | 4 | 68.95 | 14.78 | 8 | −27.61 | 8.45 | 9 |
| baseline | 6 | 83.11 | 33.75 | 7 | −41.02 | 9.49 | 9 |

| | Treat- | VGFB877 | | | VGFB877 | | |
|---|---|---|---|---|---|---|---|
| | ment | Low dose | | | High dose | | |
| | Week | Mean | SEM | N | Mean | SEM | N |
| UACR | 0 | 10226.1 | 1050.6 | 10 | 10227.5 | 1053.1 | 10 |
| (μg/mg) | 2 | 17025.3 | 1375.5 | 10 | 13345.1 | 1628.5 | 10 |
| | 4 | 16825.9 | 2580.6 | 9 | 11995.2 | 2082.7 | 9 |
| | 6 | 13060.8 | 2065.8 | 8 | 11539.5 | 3325.2 | 9 |
| % Change | 2 | 83.01 | 26.02 | 10 | 36.78 | 18.13 | 10 |
| from | 4 | 62.38 | 24.68 | 9 | 15.09 | 17.45 | 9 |
| baseline | 6 | 28.25 | 20.14 | 8 | 6.42 | 28.70 | 9 |

TABLE 39
Plasma PlGF level (pg/mL, Mean ± SEM) at week
4 and 6 post doses of VGFB877 in ReninAAV uNx db/db mice

| | Treatment | | ACEi | VGFB877 | VGFB877 |
|---|---|---|---|---|---|
| | Week | Vehicle | 10 mg/kg | Low dose | High dose |
| PlGF | 2 | 68.22 ± | 43.01 ± | 65.48 ± | 202.93 ± |
| | | 6.45 | 4.04 | 6.54 | 41.83 |
| | 4 | 55.57 ± | 62.15 ± | 568.27 ± | 693.46 ± |
| | | 5.77 | 16.45 | 160.03 | 47.53 |

For the efficacy study of mouse reformatted VGFB78 (VGFB876), ReninAAV uNx db/db mice were dosed at 3 and 10 mg/kg TW SC for 4 weeks. Table 40-41 showed UACR and the percentage change from the baseline at week 2 and 4 post doses. Table 39 showed plasma PlGF level at week 4 post doses. In contrast to the progressive increase in UACR in vehicle and isotype treated mice, VGFB876 significantly reduced the UACR at week 4 in comparison to the control groups. The low dose at 3 mg/kg reduced the UACR by −9% from baseline, and the high dose at 10 mg/kg by −20%, which is comparable to ACEi. Consistent with the significant PD effect (UACR reduction), VGFB876 induced a robust, dose-related increase in plasma PlGF level, in line with its high binding potency to mouse VEGFR1.

TABLE 40
Effect of reformatted VGFB78 (VGFB876) in ReninAAV
uNx db/db mice on UACR and % change from baseline

| | | | | | ACEi | | | Isotype | | |
|---|---|---|---|---|---|---|---|---|---|---|
| | Treatment | Vehicle | | | 10 mg/kg | | | 10 mg/kg, TW | | |
| | Week | Mean | SEM | N | Mean | SEM | N | Mean | SEM | N |
| UACR | 0 | 15039.8 | 1269.5 | 15 | 14938.3 | 1439.9 | 9 | 14977 | 4245 | 9 |
| (μg/mg) | 2 | 21260.6 | 2103.7 | 13 | 11590.9 | 2173.9 | 9 | 22750 | 4740 | 8 |
| | 4 | 21718.7 | 2085.3 | 13 | 9576.5 | 2193.4 | 8 | 21737 | 5886 | 8 |
| % Change | 2 | 48.48 | 20.56 | 13 | −8.82 | 25.86 | 9 | 65.08 | 25.19 | 8 |
| from baseline | 4 | 56.56 | 18.32 | 13 | −22.73 | 25.29 | 8 | 65.84 | 30.87 | 8 |

| | | VGFB876 | | | VGFB876 | | |
|---|---|---|---|---|---|---|---|
| | Treatment | 3 mg/kg, TW | | | 10 mg/kg, TW | | |
| | Week | Mean | SEM | N | Mean | SEM | N |
| UACR | 0 | 14475.7 | 1072.7 | 15 | 14789.1 | 1126.0 | 15 |
| (μg/mg) | 2 | 17837.8 | 2107.8 | 14 | 18585.2 | 2071.2 | 14 |
| | 4 | 11703.3 | 1468.7 | 14 | 10608.5 | 1263.2 | 14 |
| % Change | 2 | 33.41 | 20.58 | 14 | 40.60 | 21.90 | 14 |
| from baseline | 4 | −8.88 | 18.88 | 14 | −19.60 | 11.50 | 14 |

TABLE 41
Plasma PlGF level (pg/mL, Mean ± SEM) at week
4 post doses of VGFB876 in ReninAAV uNx db/db mice

| | | ACEi | Isotype | VGFB78 | VGFB78 |
|---|---|---|---|---|---|
| Treatment | | 10 mg/ | 10 mg/ | 3 mg/ | 10 mg/ |
| Week | Vehicle | kg | kg, TW | kg, TW | kg, TW |
| 4 | 82.81 ± | 69.45 ± | 74.09 ± | 1586.45 ± | 3834.22 ± |
| | 5.25 | 1.85 | 1.62 | 129.62 | 150.59 |

1. An isolated antibody or antigen binding fragment thereof that binds to an epitope within the amino acid sequence of SEQ ID NO: 173 or SEQ ID NO: 4, and wherein the antibody or antigen binding fragment thereof prevents the binding of VEGFA to VEGFR1.

2. The isolated antibody or antigen binding fragment thereof of claim 1, wherein the antibody or antigen binding fragment thereof binds to epitopes on VEGFR1 having the amino acid sequence FPLDTL (SEQ ID NO: 143) or EIGL (SEQ ID NO: 144) or binds to the epitopes having the amino acid sequences FPLDTL (SEQ ID NO: 143) and EIGL (SEQ ID NO: 144).

3. The isolated antibody or antigen binding fragment thereof of claim 1 or 2 wherein the antibody or antigen binding fragment thereof binds to human, mouse, rat and/or cynomolgus monkey VEGFR1.

4. The isolated antibody or antigen binding fragment thereof of claim 3, wherein the antibody or antigen binding fragment thereof binds human VEGFR1.

5. The isolated antibody or antigen binding fragment thereof of claim 4, wherein the antibody or antigen binding fragment thereof binds to human VEGFR1 and a VEGFR1 from at least one species selected from the group consisting of cynomolgus monkey, mouse and rat.

6. The isolated antibody or antigen binding fragment thereof of claim 5, wherein the antibody or antigen binding fragment thereof binds to human VEGFR1 and a VEGFR1 from at least two species selected from the group consisting of cynomolgus monkey, mouse and rat.

7. The isolated antibody or antigen binding fragment thereof of claim 6, wherein the antibody or antigen binding fragment thereof binds to human VEGFR1 and a VEGFR1 from cynomolgus monkey, mouse and rat.

8. The isolated antibody or antigen binding fragment thereof of any one of claims 1-7, wherein the antibody or antigen binding fragment thereof binds to a human VEGFR1, mouse VEGFR1, and cynomolgus monkey VEGFR1, with a K_{D} of 6×10⁻⁸ M or less, particularly 1×10⁻⁸ M or less, more particularly 5×10⁻⁹ M or less, 1×10⁻⁹ M or less, 5×10⁻¹⁰ M or less, or 1×10⁻¹⁰ M or less, as determined by using surface plasmon resonance (SPR).

9. The isolated antibody or antigen binding fragment thereof of any one of claims 1-8, comprising the heavy chain complementarity determining regions (HCDRs) of a VH of SEQ ID NO: 31, 33, 34, 36, 38 or 39, and the light chain complementarity determining regions (LCDRs) of a VL of SEQ ID NO: 32, 35, 37 or 40.

10. The isolated antibody or antigen binding fragment thereof of claim 9, comprising:
a. the HCDRs of the VH having the amino acid sequence of SEQ ID NO: 31, and the LCDRs of the VL having the amino acid sequence of SEQ ID NO: 32;
b. the HCDRs of the VH having the amino acid sequence of SEQ ID NO: 33, and the LCDRs of the VL having the amino acid sequence of SEQ ID NO: 32;
c. the HCDRs of the VH having the amino acid sequence of SEQ ID NO: 34, and the LCDRs of the VL having the amino acid sequence of SEQ ID NO: 35;
d. the HCDRs of the VH having the amino acid sequence of SEQ ID NO: 36, and the LCDRs of the VL having the amino acid sequence of SEQ ID NO: 37;
e. the HCDRs of the VH having the amino acid sequence of SEQ ID NO: 38, and the LCDRs of the VL having the amino acid sequence of SEQ ID NO: 37; or
f. the HCDRs of the VH having the amino acid sequence of SEQ ID NO: 39, and the LCDRs of the VL having the amino acid sequence of SEQ ID NO: 40.

11. The isolated antibody or antigen binding fragment thereof of claim 10, comprising a VH having a HCDR1, a HCDR2 and a HCDR3, and a VL having a LCDR1, a LCDR2, and a LCDR3, wherein the HCDR1, HCDR2, HCDR3, LCDR1, LCDR2 and LCDR3 comprise the amino acid sequences of:
a. SEQ ID NOs: 7, 175, 9, 10, 11 and 12, respectively;
b. SEQ ID NOs: 7, 8, 9, 10, 11 and 12, respectively;
c. SEQ ID NOs: 13, 14, 15, 16, 17 and 18, respectively;
d. SEQ ID NOs: 19, 20, 21, 22, 23 and 24, respectively;
e. SEQ ID NOs: 25, 26, 27, 28, 29, and 30, respectively;
f. SEQ ID NOs: 71, 176, 73, 74, 75 and 76, respectively;
g. SEQ ID NOs: 71, 72, 73, 74, 75 and 76, respectively;
h. SEQ ID NOs: 77, 78, 79, 80, 81, and 82, respectively;
i. SEQ ID NOs: 83, 84, 85, 86, 87, and 88, respectively;
j. SEQ ID NOs: 89, 90, 91, 92, 93 and 94, respectively;
k. SEQ ID NOs: 95, 177, 97, 98, 99 and 100, respectively
l. SEQ ID NOs: 95, 96, 97, 98, 99 and 100, respectively;
m. SEQ ID NOs: 101, 102, 103, 104, 105 and 106, respectively;
n. SEQ ID NOs: 107, 108, 109, 110, 111, and 112, respectively;
o. SEQ ID NOs: 113, 114, 115, 116, 117, and 118, respectively;
p. SEQ ID NOs: 119, 178, 121, 122, amino acid sequence LNS, and SEQ ID NO: 124, respectively;
q. SEQ ID NOs: 119, 120, 121, 122, amino acid sequence LNS, and SEQ ID NO: 124, respectively;
r. SEQ ID NOs: 125, 126, 127, 128, amino acid sequence FNF, and SEQ ID NO: 130, respectively;
s. SEQ ID NOs: 131, 132, 133, 134, amino acid sequence YD, and SEQ ID NO: 136, respectively; or
t. SEQ ID NOs: 137, 138, 139, 140, amino acid sequence FNS, and SEQ ID NO: 142, respectively.

12. The isolated antibody or antigen binding fragment thereof of claim 10, wherein the HCDR1, HCDR2, HCDR3, LCDR1, LCDR2 and LCDR3 comprise the amino acid sequences of SEQ ID NOs: 7, 175, 9, 10, 11 and 12, respectively.

13. The isolated antibody or antigen binding fragment thereof of claim 11, wherein the HCDR1, HCDR2, HCDR3, LCDR1, LCDR2 and LCDR3 comprise the amino acid sequences of SEQ ID NOs: 13, 14, 15, 16, 17, and 18, respectively.

14. The isolated antibody or antigen binding fragment thereof of claim 11, wherein the HCDR1, HCDR2, HCDR3, LCDR1, LCDR2 and LCDR3 comprise the amino acid sequences of SEQ ID NOs: 25, 26, 27, 28, 29 and 30, respectively.

15. The isolated antibody or antigen binding fragment thereof of claim 11, wherein the HCDR1, HCDR2, HCDR3, LCDR1, LCDR2 and LCDR3 comprise the amino acid sequences of SEQ ID NOs: 7, 8, 9, 10, 11, and 12, respectively.

16. The isolated antibody or antigen binding fragment thereof of claim 11, wherein the HCDR1, HCDR2, HCDR3, LCDR1, LCDR2 and LCDR3 comprise the amino acid sequences of SEQ ID NOs: 19, 20, 21, 22, 23, and 24, respectively.

17. The isolated antibody or antigen binding fragment thereof of any one of claims 1-16, comprising a heavy chain variable domain (VH) having an amino acid sequence at least 80% (e.g. at least 85%, at least 90%, at least 95%, or at least 99%) identical to the amino acid sequence of SEQ ID NO: 31, 33, 34, 36, 38 or 39, and a light chain variable domain (VL) having an amino acid sequence at least 80% (e.g. at least 85%, at least 90%, at least 95%, or at least 99%) identical to the amino acid sequence of SEQ ID NO: 32, 35, 37 or 40.

18. The isolated antibody or antigen binding fragment thereof of claim 17, comprising a VH and a VL comprising the amino acid sequences of:
a. SEQ ID NOs: 31 and 32, respectively;
b. SEQ ID NOs: 33 and 32, respectively;
c. SEQ ID NOs: 34 and 35, respectively;
d. SEQ ID NOs: 36 and 37, respectively;
e. SEQ ID NOs: 38 and 37, respectively; or
f. SEQ ID NOs: 39 and 40, respectively.

19. The isolated antibody or antigen binding fragment thereof of claim 18, comprising the VH which is at least 80% (e.g. at least 85%, at least 90%, at least 95%, at least 99% or 100%) identical to the amino acid sequence of SEQ ID NO: 39, and the VL which is at least 80% (e.g. at least 85%, at least 90%, at least 95%, at least 99% or 100%) identical to the amino acid sequence of SEQ ID NO: 40.

20. The isolated antibody or antigen binding fragment thereof of claim 19, comprising the VH having the amino acid sequence of SEQ ID NO: 39 and the VL having the amino acid sequence of SEQ ID NO: 40.

21. The isolated antibody or antigen binding fragment thereof of claim 18, comprising the VH which is at least 80% (e.g. at least 85%, at least 90%, at least 95%, at least 99% or 100%) identical to the amino acid sequence of SEQ ID NO: 33 and a VL which is at least 80% (e.g. at least 85%, at least 90%, at least 95%, at least 99% or 100%) identical to the amino acid sequence of SEQ ID NO: 32.

22. The isolated antibody or antigen binding fragment thereof of claim 21, comprising the VH comprising the amino acid sequence of SEQ ID NO: 33 and the VL comprising the amino acid sequence of SEQ ID NO: 32.

23. The isolated antibody or antigen binding fragment thereof of claim 18, comprising the VH which is at least 80% (e.g. at least 85%, at least 90%, at least 95%, at least 99% or 100%) identical to the amino acid sequence of SEQ ID NO: 38 and the VL which is at least 80% (e.g. at least 85%, at least 90%, at least 95%, at least 99% or 100%) identical to the amino acid sequence of SEQ ID NO: 37.

24. The isolated antibody or antigen binding fragment thereof of claim 23, comprising the VH comprising the amino acid sequence of SEQ ID NO: 38 and the VL comprising the amino acid sequence of SEQ ID NO: 37.

25. The isolated antibody or antigen binding fragment thereof of claim 18, comprising the VH which is at least 80% (e.g. at least 85%, at least 90%, at least 95%, at least 99% or 100%) identical to the amino acid sequence of SEQ ID NO: 31 and the VL which is at least 80% (e.g. at least 85%, at least 90%, at least 95%, at least 99% or 100%) identical to the amino acid sequence of SEQ ID NO: 32.

26. The isolated antibody or antigen binding fragment thereof of claim 25, comprising the VH comprising the amino acid sequence of SEQ ID NO: 31 and the VL comprising the amino acid sequence of SEQ ID NO: 32.

27. The isolated antibody or antigen binding fragment thereof of claim 18, comprising the VH which is at least 80% (e.g. at least 85%, at least 90%, at least 95%, at least 99% or 100%) identical to the amino acid sequence of SEQ ID NO: 36 and the VL which is at least 80% (e.g. at least 85%, at least 90%, at least 95%, at least 99% or 100%) identical to the amino acid sequence of SEQ ID NO: 37.

28. The isolated antibody or antigen binding fragment thereof of claim 27, comprising the VH comprising the amino acid sequence of SEQ ID NO: 36 and the VL comprising the amino acid sequence of SEQ ID NO: 37.

29. The isolated antibody or antigen binding fragment thereof of claim 18, comprising the VH which is at least 80% (e.g. at least 85%, at least 90%, at least 95%, at least 99% or 100%) identical to the amino acid sequence of SEQ ID NO: 34 and the VL which is at least 80% (e.g. at least 85%, at least 90%, at least 95%, at least 99% or 100%) identical to the amino acid sequence of SEQ ID NO: 35.

30. The isolated antibody or antigen binding fragment thereof of claim 29, comprising the VH comprising the amino acid sequence of SEQ ID NO: 34 and the VL comprising the amino acid sequence of SEQ ID NO: 35.

31. The isolated antibody or antigen binding fragment thereof of any one of claims 1-30, comprising a heavy chain (HC) having an amino acid sequence at least 80% (e.g. at least 85%, at least 90%, at least 95%, or at least 99%) identical to the amino acid sequence of SEQ ID NO: 51, SEQ ID NO: 53, SEQ ID NO: 54, SEQ ID NO: 56, SEQ ID NO: 58 or SEQ ID NO: 59, and a light chain (LC) having an amino acid sequence at least 80% (e.g. at least 85%, at least 90%, at least 95%, or at least 99%) identical to the amino acid sequence of SEQ ID NO: 52, SEQ ID NO: 55, SEQ ID NO: 57 or SEQ ID NO: 60.

32. The isolated antibody or antigen binding fragment thereof of claim 31 comprising an amino acid sequence selected from the group consisting of SEQ ID NOs: 51, 52, 53, 54, 55, 56, 57, 59 and 60.

33. The isolated antibody of claim 32, comprising
a. the HC comprising the amino acid sequence at least 80% (e.g. at least 85%, at least 90%, at least 95%, at least 99% or 100%) identical to the amino acid sequence of SEQ ID NO: 51, and the LC comprising the amino acid sequence at least 80% (e.g. at least 85%, at least 90%, at least 95%, at least 99% or 100%) identical to the amino acid sequence of SEQ ID NO: 52;
b. the HC comprising the amino acid sequence at least 80% (e.g. at least 85%, at least 90%, at least 95%, at least 99% or 100%) identical to the amino acid sequence of SEQ ID NO: 53, and the LC comprising the amino acid sequence at least 80% (e.g. at least 85%, at least 90%, at least 95%, at least 99% or 100%) identical to the amino acid sequence of SEQ ID NO: 52;
c. the HC comprising the amino acid sequence at least 80% (e.g. at least 85%, at least 90%, at least 95%, at least 99% or 100%) identical to the amino acid sequence of SEQ ID NO: 54, and the LC comprising the amino acid sequence at least 80% (e.g. at least 85%, at least 90%, at least 95%, at least 99% or 100%) identical to the amino acid sequence of SEQ ID NO: 55;
d. the HC comprising the amino acid sequence at least 80% (e.g. at least 85%, at least 90%, at least 95%, at least 99% or 100%) identical to the amino acid sequence of SEQ ID NO: 56, and the LC comprising the amino acid sequence at least 80% (e.g. at least 85%, at least 90%, at least 95%, at least 99% or 100%) identical to the amino acid sequence of SEQ ID NO: 57;
e. the HC comprising the amino acid sequence at least 80% (e.g. at least 85%, at least 90%, at least 95%, at least 99% or 100%) identical to the amino acid sequence of SEQ ID NO: 58, and the LC comprising the amino acid sequence at least 80% (e.g. at least 85%, at least 90%, at least 95%, at least 99% or 100%) identical to the amino acid sequence of SEQ ID NO: 57; or
f. the HC comprising the amino acid sequence at least 80% (e.g. at least 85%, at least 90%, at least 95%, at least 99% or 100%) identical to the amino acid sequence of SEQ ID NO: 59, and the LC comprising the amino acid sequence at least 80% (e.g. at least 85%, at least 90%, at least 95%, at least 99% or 100%) identical to the amino acid sequence of SEQ ID NO: 60.

34. The isolated antibody of claim 33, comprising the HC comprising the amino acid sequence of SEQ ID NO: 51 and the LC comprising the amino acid sequence of SEQ ID NO: 52.

35. The isolated antibody of claim 33, comprising the HC comprising the amino acid sequence of SEQ ID NO: 53 and the LC comprising the amino acid sequence of SEQ ID NO: 52.

36. The isolated antibody of claim 33, comprising the HC comprising the amino acid sequence of SEQ ID NO: 54 and the LC comprising the amino acid sequence of SEQ ID NO: 55.

37. The isolated antibody of claim 33, comprising the HC comprising the amino acid sequence of SEQ ID NO: 56 and the LC comprising the amino acid sequence of SEQ ID NO: 57.

38. The isolated antibody of claim 33, comprising the HC comprising the amino acid sequence of SEQ ID NO: 58 and the LC comprising the amino acid sequence of SEQ ID NO: 57.

39. The isolated antibody of claim 33, comprising the HC comprising the amino acid sequence of SEQ ID NO: 59 and the LC comprising the amino acid sequence of SEQ ID NO: 60.

40. An isolated antibody or antigen binding fragment thereof, comprising
a. the HCDRs of a VH comprising the amino acid sequence of SEQ ID NO: 33, and the LCDRs of a VL comprising the amino acid sequence of SEQ ID NO: 32;
b. a VH comprising a HCDR1, a HCDR2, and a HCDR3 having an amino acid sequence of SEQ ID NOs: 7, 8, and 9, respectively, and a VL comprising a LCDR1, LCDR2, and LCDR3 having an amino acid sequence of SEQ ID NOs: 10, 11, and 12, respectively;
c. a VH having an amino acid sequence of SEQ ID NO: 33 and a VL having an amino acid sequence of SEQ ID NO: 32; and/or
d. a HC having an amino acid sequence of SEQ ID NO: 53; and a LC having an amino acid sequence of SEQ ID NO: 52.

41. An isolated antibody or antigen binding fragment thereof, comprising
a. the HCDRs of a VH comprising the amino acid sequence of SEQ ID NO: 34, and the LCDRs of a VL comprising the amino acid sequence of SEQ ID NO: 35;
b. a VH comprising a HCDR1, a HCDR2, and a HCDR3 having an amino acid sequence of SEQ ID NOs: 13, 14, and 15, respectively, and a VL comprising a LCDR1, LCDR2, and LCDR3 having an amino acid sequence of SEQ ID NOs: 16, 17 and 18, respectively;
c. a VH having an amino acid sequence of SEQ ID NO: 34 and a VL having an amino acid sequence of SEQ ID NO: 35; and/or d. HC having an amino acid sequence of SEQ ID NO: 54; and a LC having an amino acid sequence of SEQ ID NO: 55

42. An isolated antibody or antigen binding fragment thereof, comprising
a. a heavy chain variable region (VH) having the HCDRs of a VH comprising the amino acid sequence of SEQ ID NO: 38, and light chain variable region (VL) having the LCDRs of a VL comprising the amino acid sequence of SEQ ID NO: 37;
b. a VH comprising a HCDR1, a HCDR2, and a HCDR3 having an amino acid sequence of SEQ ID NOs: 19, 20, and 21, respectively, and a VL comprising a LCDR1, LCDR2, and LCDR3 having an amino acid sequence of SEQ ID NOs: 22, 23, and 24, respectively;
c. a VH having an amino acid sequence of SEQ ID NO: 38 and a VL having an amino acid sequence of SEQ ID NO: 37; and/or
d. a HC having an amino acid sequence of SEQ ID NO: 58; and a LC having an amino acid sequence of SEQ ID NO: 57.

43. An isolated antibody or antigen binding fragment thereof, comprising
a. a heavy chain variable region (VH) having the HCDRs of a VH comprising the amino acid sequence of SEQ ID NO: 39, and light chain variable region (VL) having the LCDRs of a VL comprising the amino acid sequence of SEQ ID NO: 40;
b. a VH comprising a HCDR1, a HCDR2, and a HCDR3 having an amino acid sequence of SEQ ID NOs: 25, 26, and 27, respectively, and a VL comprising a LCDR1, LCDR2, and LCDR3 having an amino acid sequence of SEQ ID NOs: 28, 29, and 30, respectively;
c. a VH having an amino acid sequence of SEQ ID NO: 39 and a VL having an amino acid sequence of SEQ ID NO: 40; and/or
d. a HC having an amino acid sequence of SEQ ID NO: 59 and a LC having an amino acid sequence of SEQ ID NO: 60.

44. An isolated antibody or antigen binding fragment thereof, comprising
a. a heavy chain variable region (VH) having the HCDRs of a VH comprising the amino acid sequence of SEQ ID NO: 31, and light chain variable region (VL) having the LCDRs of a VL comprising the amino acid sequence of SEQ ID NO: 32;
b. a VH comprising a HCDR1, a HCDR2, and a HCDR3 having an amino acid sequence of SEQ ID NOs: 7, 175, and 9, respectively, and a VL comprising a LCDR1, LCDR2, and LCDR3 having an amino acid sequence of SEQ ID NOs: 10, 11, and 12, respectively;
c. a VH having an amino acid sequence of SEQ ID NO: 31 and a VL having an amino acid sequence of SEQ ID NO: 32; and/or
d. a HC having an amino acid sequence of SEQ ID NO: 51 and a LC having an amino acid sequence of SEQ ID NO: 52.

45. An isolated antibody or antigen binding fragment thereof, comprising
a. a heavy chain variable region (VH) having the HCDRs of a VH comprising the amino acid sequence of SEQ ID NO: 36, and light chain variable region (VL) having the LCDRs of a VL comprising the amino acid sequence of SEQ ID NO: 37;
b. a VH comprising a HCDR1, a HCDR2, and a HCDR3 having an amino acid sequence of SEQ ID NOs: 19, 20 and 21, respectively, and a VL comprising a LCDR1, LCDR2, and LCDR3 having an amino acid sequence of SEQ ID NOs: 22, 23, and 24, respectively;
c. a VH having an amino acid sequence of SEQ ID NO: 36 and a VL having an amino acid sequence of SEQ ID NO: 37; and/or
d. a HC having an amino acid sequence of SEQ ID NO: 56 and a LC having an amino acid sequence of SEQ ID NO: 57.

46. The isolated antigen binding fragment of any one of claims 1-45, wherein the antigen binding fragment is a scFv, a (scFv)₂, a Fv, a Fab, a F(ab′)₂, a Fd, a dAb, a VHH or single chain antibody.

47. The isolated antibody or antigen binding fragment thereof of any one of claims 1-46, wherein the antibody or antigen binding fragment thereof is of an IgG1, an IgG2, an IgG3 or an IgG4 isotype.

48. The isolated antibody or antigen binding fragment thereof of claim 47, wherein the antibody or antigen binding fragment thereof is an IgG1 isotype.

49. The isolated antibody or antigen binding fragment thereof of claim 48, comprising an Ig constant region or a fragment of the Ig constant region.

50. The isolated antibody or antigen binding fragment thereof of claim 49, wherein the Ig constant region or the fragment of the Ig constant region comprises at least one mutation that results in reduced binding of the antibody or antigen binding fragment thereof to a Fcγ receptor (FcγR).

51. The isolated antibody or antigen binding fragment thereof of claim 50, wherein the at least one mutation that results in reduced binding of the antibody or antigen binding fragment thereof to a FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S and S228P/F234A/L235A/G236-deleted/G237A/P238S, wherein residue numbering is according to the EU index.

52. The isolated antibody or antigen binding fragment thereof of claim 51, wherein the Ig constant region or the fragment of the constant region comprises the mutations of L234A_L235A_D265S.

53. The isolated antibody or antigen binding fragment thereof of claim 52, wherein the FcγR is FcγRI, FcγRIIA, FcγRIIB or FcγRIII, or any combination thereof.

54. The isolated antibody or antigen binding fragment thereof of claim 53, wherein the Ig constant region or the fragment of the Ig constant region further comprises at least one mutation that modulates a half-life of the antibody or antigen binding fragment thereof.

55. The isolated antibody or antigen binding fragment thereof of claim 54, wherein the at least one mutation that modulates the half-life of the antibody or antigen binding fragment thereof is selected from the group consisting of H435A, P257I/N434H, D376V/N434H, M252Y/S254T/T256E/H433K/N434F, T308P/N434A and H435R, wherein residue numbering is according to the EU index.

56. An immunoconjugate comprising the isolated antibody or antigen binding fragment thereof of any one of claims 1-55 conjugated to a therapeutic agent or an imaging agent.

57. A pharmaceutical composition comprising the isolated antibody or antigen binding fragment thereof of any one of claims 1-55 or the immunoconjugate of claim 56, and a pharmaceutically acceptable carrier.

58. An isolated polynucleotide encoding the isolated antibody or antigen binding fragment thereof of any one of claims 1-55.

59. The isolated polynucleotide of claim 58, comprising a polynucleotide sequence at least 80%, such as at least 85%, at least 90%, at least 95%, at least 99% or 100%, identical to the polynucleotide sequence of SEQ ID NOs: 41, 42, 43, 44, 45, 46, 47, 48, 49, 50, 61, 62, 63, 64, 65, 66, 67, 68, 69, or 70.

60. A vector comprising the polynucleotide of claim 58 or 59.

61. A host cell expressing the isolated antibody or antigen binding fragment thereof of any one of claims 1-55, such as a host cell comprising the vector of claim 60.

62. A method of preventing the binding of VEGFA to a VEGFR1 in a subject in need thereof, comprising administering to the subject an effective amount of the isolated antibody or antigen fragment thereof of any one of claims 1-55, the immunoconjugate of claim 56, the pharmaceutical composition of claim 57, the isolated polynucleotide of claim 58 or 59, the vector of claim 60 or the host cell of claim 61 to thereby prevent the binding of VEGFA to the VEGFR1.

63. The method of claim 62, wherein the subject is in need of a treatment of a Chronic Kidney Disease (CKD), in need of decreasing proteinuria, has advanced stage 4 or 5 chronic kidney disease, or has CKD with proteinuria, albuminuria, or diabetes mellitus.

64. A method of treating a Chronic Kidney Disease (CKD) in a subject in need thereof, comprising administering a therapeutically effective amount of the isolated antibody or antigen fragment thereof of any one of claims 1-55, the immunoconjugate of claim 56, the pharmaceutical composition of claim 57, the isolated polynucleotide of claim 58 or 59, the vector of claim 60, or the host cell of claim 61 to the subject for a time sufficient to treat the CKD.

65. The method of claim 64, wherein the subject has advanced stage 4 or 5 chronic kidney disease.

66. The method of claim 64, wherein the subject has stage 3 chronic kidney disease.

67. The method of claim 64, wherein the subject has CKD with proteinuria, albuminuria, or diabetes mellitus.

68. A method of decreasing proteinuria in a subject in need thereof, comprising administering a therapeutically effective amount of the isolated antibody or antigen fragment thereof of any one of claims 1-55, the immunoconjugate of claim 56, the pharmaceutical composition of claim 57, the isolated polynucleotide of claim 58 or 59, the vector of claim 60, or the host cell of claim 59, to the subject for a time sufficient to decrease proteinuria in the subject.

69. A kit comprising the isolated antibody or antigen binding fragment thereof of any one of claims 1-55, the immunoconjugate of claim 56, the pharmaceutical composition of claim 57, the isolated polynucleotide of claim 58 or 59, the vector of claim 60, or the host cell of claim 61.


SEQUENCE LISTING (186 sequences)

SEQ ID NO: 1 - protein, 447 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKLEVLFQGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 2 - protein, 555 aa, synthetic construct
YGSGSKLKVPELSLKGTQHVMQAGQTLFLKCRGEAAHSWSLPTTVSQEDKRLSITPPSACGRDNRQFCSTLTLDTAQANHTGLYTCRYLPTSTSKKKKAESSIYIFVSDAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEKLEVLFQGPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 3 - protein, 117 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQGLNDIFEAQKIEWHEHHHHHH

SEQ ID NO: 4 - protein, 223 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKGLNDIFEAQKIEWHEHHHHHH

SEQ ID NO: 5 - protein, 331 aa, synthetic construct
YGSGSKLKVPELSLKGTQHVMQAGQTLFLKCRGEAAHSWSLPTTVSQEDKRLSITPPSACGRDNRQFCSTLTLDTAQANHTGLYTCRYLPTSTSKKKKAESSIYIFVSDAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEKGLNDIFEAQKIEWHEHHHHHH

SEQ ID NO: 6 - protein, 753 aa, synthetic construct
SKLKGPELSLKGTQHVTQAGQTLHLQCRGEAAHQWSLPETVSKESKRLSITKSACGRNGKQFCSTLTLNAAQANHTGFYSCKYLAVPTSKKKETESAIYIFISDTGRPFVEMYSEIPEIIHMTEGRELIIPCRVTSPNITVTLKKFPLDTLIPDGKRVIWDSRKGFIISNATYKEIGLLTCEATVNGHLYKTNYLTHRQTNTIIDVQISTPRPVKLLRGHTLILNCTATTPLNTRVQMTWSYPDEKNKRASVRRRIDQSNSHANIFYSVLTIDKVQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPRPTIKWCWRPCNHNHSEARYDFCSNNEESFILDPDSNIGNRIESITQRMAIIEGKNKTASTLVVADSRISGIYSCIASNKVGTVGRNISFYITDVPNGFHVNLEKMPTEGEDLKLSCTVNKFLYRDVTWILLRTVNNRTMHYSISKQKMAITKEHSITLNLTIMNVSLEDSGTYACRARNVYTGEEILQKKEVTIRDQEAPYLLRNLSDHTVAISSSTTLDCHANGAPEPQITWFKNNHKIQQEPGIILGPGSSTLFIERVTEEDEGVYHCKATNQKGSVESSAYLTVQGTSDKSNLEGLNDIFEAQKIEWHEHHHHHH

SEQ ID NO: 7 - protein, 5 aa, synthetic construct
DYGMN

SEQ ID NO: 8 - protein, 18 aa, synthetic construct
SINRQGDISTYHVDSVKG

SEQ ID NO: 9 - protein, 9 aa, synthetic construct
NHDYHGMDV

SEQ ID NO: 10 - protein, 13 aa, synthetic construct
SGSRSNIGNNFVN

SEQ ID NO: 11 - protein, 7 aa, synthetic construct
LNSQRPS

SEQ ID NO: 12 - protein, 11 aa, synthetic construct
ASWDDSLNGWV

SEQ ID NO: 13 - protein, 5 aa, synthetic construct
DYNMN

SEQ ID NO: 14 - protein, 17 aa, synthetic construct
IITNDGSSTAYSDSVKG

SEQ ID NO: 15 - protein, 7 aa, synthetic construct
VYYGMDD

SEQ ID NO: 16 - protein, 13 aa, synthetic construct
SGGSSNIGSNYVS

SEQ ID NO: 17 - protein, 7 aa, synthetic construct
FNFQRPS

SEQ ID NO: 18 - protein, 11 aa, synthetic construct
AAWDDRVNVWV

SEQ ID NO: 19 - protein, 5 aa, synthetic construct
NYDMT

SEQ ID NO: 20 - protein, 17 aa, synthetic construct
GIGTSGTDMYYADSVKG

SEQ ID NO: 21 - protein, 9 aa, synthetic construct
DYYYYGMDV

SEQ ID NO: 22 - protein, 13 aa, synthetic construct
SGSTSNIGNNYVS

SEQ ID NO: 23 - protein, 6 aa, synthetic construct
YDQRPS

SEQ ID NO: 24 - protein, 11 aa, synthetic construct
AAWDDSLNGWV

SEQ ID NO: 25 - protein, 5 aa, synthetic construct
TYAMN

SEQ ID NO: 26 - protein, 17 aa, synthetic construct
TINSEGTITSHAPAVKG

SEQ ID NO: 27 - protein, 10 aa, synthetic construct
TTGTTHGMDV

SEQ ID NO: 28 - protein, 13 aa, synthetic construct
SGSSSNIQNNYVY

SEQ ID NO: 29 - protein, 7 aa, synthetic construct
FNSQRPS

SEQ ID NO: 30 - protein, 11 aa, synthetic construct
GTWDDSLNGWV

SEQ ID NO: 31 - protein, 119 aa, synthetic construct
EVQLLESGGGSVQPGGSLRLSCTASGFNFEDYGMNWVRQAPGKGLDWVSSINRNGDISTYHVDSVKGRFTISRDNSKNTLYLQMNSLRAEDTAIYYCTKNHDYHGMDVWGQGTTVTVSS

SEQ ID NO: 32 - protein, 110 aa, synthetic construct
QSVLTQPPSASGTPGQRVTISCSGSRSNIGNNFVNWYQQIPGTAPKLLIYLNSQRPSGVPDRFSGSKSGTSASLAISGLQSDDEADYYCASWDDSLNGWVFGGGTKLTVL

SEQ ID NO: 33 - protein, 119 aa, synthetic construct
EVQLLESGGGSVQPGGSLRLSCTASGFNFEDYGMNWVRQAPGKGLDWVSSINRQGDISTYHVDSVKGRFTISRDNSKNTLYLQMNSLRAEDTAIYYCTKNHDYHGMDVWGQGTTVTVSS

SEQ ID NO: 34 - protein, 116 aa, synthetic construct
EVQLLESGGGLVQPGGSLRLSCEASGFIFRDYNMNWVRQAPGKGLEWVSIITNDGSSTAYSDSVKGRFTISRDNSKNSLYLQMNSLRAEDTAIYYCVKVYYGMDDWGQGTTVTVSS

SEQ ID NO: 35 - protein, 110 aa, synthetic construct
QSVLTQPPSASGTPGQRVTISCSGGSSNIGSNYVSWYQQLPGTAPKLLIYFNFQRPSGVPDRFSGSKSGTSASLAISGLQSEDEADYYCAAWDDRVNVWVFGGGTKLTVL

SEQ ID NO: 36 - protein, 118 aa, synthetic construct
EVQLLESGGGLVQPGGSLRLSCAASSFTFYNYDMTWVRQAPGKGLEWVAGIGTSGTDMYYADSVKGRFTISRDNSKNTLYLQMNSLRAEDTAIYYCAKDYYYYGMDVWGLGTTVTVSS

SEQ ID NO: 37 - protein, 109 aa, synthetic construct
QSVLTQPPSASGTPGQRVIISCSGSTSNIGNNYVSWYQQLPGTAPKLLIYYDQRPSGVPDRFSGSKSGTSASLAISGLQSEDEADYYCAAWDDSLNGWVFGGGTKLTVL

SEQ ID NO: 38 - protein, 118 aa, synthetic construct
EVQLLESGGGLVQPGGSLRLSCAASSFTFYNYDMTWVRQAPGKGLEWVAGIGTSGTDMYYADSVKGRFTISRDNSKNTLYLQMNSLRAEDTAIYYCAKDYYYYGMDVWGQGTTVTVSS

SEQ ID NO: 39 - protein, 119 aa, synthetic construct
EVQLLESGGGLVQPGGSLRLSCEASGFDFSTYAMNWVRQAPGKGLEWVATINSEGTITSHAPAVKGRFTISRDNSKNTAYVQMNSLRAEDSAVYYCSSTTGTTHGMDVWGRGTTVTVSS

SEQ ID NO: 40 - protein, 110 aa, synthetic construct
QSVLTQPPSASGTPGQRVTISCSGSSSNIQNNYVYWYQQLPGTAPKLLIYFNSQRPSGVPDRFSGSKSGTSASLAISGLQSEDEADYYCGTWDDSLNGWVFGGGTKLTVL

SEQ ID NO: 41 - DNA, 357 bp, synthetic construct
  source 1..357: mol_type = other DNA
gaggttcagctgctggaatctggcggaggatctgttcagcctggcggctctctgagactgtcttgtaccgcctccggcttcaacttcgaggactacggcatgaactgggtccgacaggctcctggcaaaggcctggattgggtgtcctccatcaaccggaacggcgacatctccacctaccacgtggactccgtgaagggcagattcaccatcagccgggacaactccaagaacaccctgtacctgcagatgaactccctgagagccgaggacaccgccatctactactgcaccaagaaccacgactaccacggcatggatgtgtggggccaaggcacaacagtgaccgtttcttct

SEQ ID NO: 42 - DNA, 330 bp, synthetic construct
  source 1..330: mol_type = other DNA
cagtctgtgctgacccagcctccttctgcttctggcacacctggccagagagtgaccatctcctgttccggctctcggtccaacatcggcaacaacttcgtgaactggtatcagcagatccccggcaccgctcctaagctgctgatctacctgaactcccagcggccttctggcgtgcccgatagattctccggctctaagtctggcacctctgccagcctggctatctctggcctgcagtctgatgacgaggccgactactactgcgcctcctgggacgattctctgaacggctgggttttcggcggaggcaccaaactgacagtgctg

SEQ ID NO: 43 - DNA, 357 bp, synthetic construct
  source 1..357: mol_type = other DNA
gaggttcagctgctggaatctggcggaggatctgttcagcctggcggctctctgagactgtcttgtaccgcctccggcttcaacttcgaggactacggcatgaactgggtccgacaggctcctggcaaaggcctggattgggtgtcctctatcaaccggcagggcgacatctccacctaccacgtggactctgtgaagggcagattcaccatcagccgggacaactccaagaacaccctgtacctgcagatgaactccctgagagccgaggacaccgccatctactactgcaccaagaaccacgactaccacggcatggatgtgtggggacaaggcacaacagtgaccgtttcttct

SEQ ID NO: 44 - DNA, 348 bp, synthetic construct
  source 1..348: mol_type = other DNA
gaggttcagctgctggaatctggcggaggattggttcagcctggcggctctctgagactgtcttgcgaggcctctggcttcatcttccgggactacaacatgaactgggtccgacaggctcctggcaaaggcctggaatgggtgtccatcatcaccaacgacggctcctccaccgcctactccgattctgtgaagggcagattcaccatcagccgggacaactccaagaactccctgtacctgcagatgaacagcctgagagccgaggacaccgccatctactactgcgtgaaggtgtactacggcatggacgattggggccagggcaccacagtgacagtctcttct

SEQ ID NO: 45 - DNA, 330 bp, synthetic construct
  source 1..330: mol_type = other DNA
cagtctgtgctgacccagcctccttctgcttctggcacacctggccagagagtgaccatctcttgctctggcggctcctccaacatcggctccaattacgtgtcctggtatcagcagctgcccggcacagctcccaaactgctgatctacttcaacttccagcggccttctggcgtgcccgacagattctctggctctaagtctggcacctctgccagcctggctatctctggactgcagagcgaggacgaggccgactactattgtgccgcctgggacgacagagtgaacgtgtgggttttcggcggaggcaccaagctgacagttctg

SEQ ID NO: 46 - DNA, 354 bp, synthetic construct
  source 1..354: mol_type = other DNA
gaagttcagcttttggagtccggtgggggattggttcagccagggggaagtctgcgattgtcatgtgcagcatcctccttcacattctataattatgacatgacatgggtacggcaggcaccaggaaaaggactggagtgggttgctgggattgggacatcaggaactgatatgtattacgcagattcagtaaagggtcgctttacaataagccgcgataatagtaaaaatacattgtatcttcaaatgaactcactcagagccgaggacactgctatttactactgcgctaaagattactattattacgggatggacgtatggggccttggcacaactgttaccgtctcaagc

SEQ ID NO: 47 - DNA, 327 bp, synthetic construct
  source 1..327: mol_type = other DNA
cagtctgtgctgacccagcctccttctgcttctggcacacctggccagagagtgatcatctcctgctccggctccacctccaacatcggcaacaactacgtgtcctggtatcagcagctgcccggcacagctcccaaactgctgatctactacgaccagcggccttctggcgtgcccgatagattctccggctctaagtctggcacctctgccagcctggctatctctggactgcagagcgaggacgaggccgactactattgtgccgcctgggacgattctctgaacggctgggttttcggcggaggcaccaaactgacagtgctg

SEQ ID NO: 48 - DNA, 354 bp, synthetic construct
  source 1..354: mol_type = other DNA
gaggttcagctgctggaatctggcggaggattggttcagcctggcggttctctgagactgtcttgtgccgcctcctccttcaccttctacaactacgacatgacctgggtccgacaggcccctggcaaaggattggaatgggtggccggcatcggcacctctggcaccgatatgtactacgccgactccgtgaagggcagattcaccatctctcgggacaactccaagaacaccctgtacctgcagatgaactccctgagagccgaggacaccgccatctactactgcgccaaggattactactactacggcatggacgtgtggggccaaggcacaacagtgacagtctcttct

SEQ ID NO: 49 - DNA, 357 bp, synthetic construct
  source 1..357: mol_type = other DNA
gaggttcagctgctggaatctggcggaggattggttcagcctggcggctctctgagactgtcttgcgaggcctctggcttcgacttctccacctacgccatgaactgggtccgacaggctcctggcaaaggcctggaatgggtcgccaccatcaattccgagggcaccatcacctctcacgcccctgctgtgaagggcagattcaccatctctcgggacaactccaagaacaccgcctacgtgcagatgaactccctgagagccgaggactccgccgtgtactactgttcttctaccaccggcaccacacacggcatggatgtgtggggacgcggcaccacagtgaccgtttcttct

SEQ ID NO: 50 - DNA, 330 bp, synthetic construct
  source 1..330: mol_type = other DNA
cagtctgtgctgacccagcctccttctgcttctggcacacctggccagagagtgaccatctcttgctccggctcctccagcaacatccagaacaactacgtgtactggtatcagcagctgcccggcaccgctcctaaactgctgatctacttcaactcccagcggccttctggcgtgcccgatagattctccggctctaagtctggcacctctgccagcctggctatctctggactgcagagcgaggacgaggccgactactattgtggcacctgggacgattccctgaacggctgggttttcggcggaggcacaaagctgacagtgctg

SEQ ID NO: 51 - protein, 449 aa, synthetic construct
EVQLLESGGGSVQPGGSLRLSCTASGFNFEDYGMNWVRQAPGKGLDWVSSINRNGDISTYHVDSVKGRFTISRDNSKNTLYLQMNSLRAEDTAIYYCTKNHDYHGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 216 aa, synthetic construct
QSVLTQPPSASGTPGQRVTISCSGSRSNIGNNFVNWYQQIPGTAPKLLIYLNSQRPSGVPDRFSGSKSGTSASLAISGLQSDDEADYYCASWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 53 - protein, 449 aa, synthetic construct
EVQLLESGGGSVQPGGSLRLSCTASGFNFEDYGMNWVRQAPGKGLDWVSSINRQGDISTYHVDSVKGRFTISRDNSKNTLYLQMNSLRAEDTAIYYCTKNHDYHGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 446 aa, synthetic construct
EVQLLESGGGLVQPGGSLRLSCEASGFIFRDYNMNWVRQAPGKGLEWVSIITNDGSSTAYSDSVKGRFTISRDNSKNSLYLQMNSLRAEDTAIYYCVKVYYGMDDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 216 aa, synthetic construct
QSVLTQPPSASGTPGQRVTISCSGGSSNIGSNYVSWYQQLPGTAPKLLIYFNFQRPSGVPDRFSGSKSGTSASLAISGLQSEDEADYYCAAWDDRVNVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 448 aa, synthetic construct
EVQLLESGGGLVQPGGSLRLSCAASSFTFYNYDMTWVRQAPGKGLEWVAGIGTSGTDMYYADSVKGRFTISRDNSKNTLYLQMNSLRAEDTAIYYCAKDYYYYGMDVWGLGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 215 aa, synthetic construct
QSVLTQPPSASGTPGQRVIISCSGSTSNIGNNYVSWYQQLPGTAPKLLIYYDQRPSGVPDRFSGSKSGTSASLAISGLQSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 58 - protein, 448 aa, synthetic construct
EVQLLESGGGLVQPGGSLRLSCAASSFTFYNYDMTWVRQAPGKGLEWVAGIGTSGTDMYYADSVKGRFTISRDNSKNTLYLQMNSLRAEDTAIYYCAKDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 449 aa, synthetic construct
EVQLLESGGGLVQPGGSLRLSCEASGFDFSTYAMNWVRQAPGKGLEWVATINSEGTITSHAPAVKGRFTISRDNSKNTAYVQMNSLRAEDSAVYYCSSTTGTTHGMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 216 aa, synthetic construct
QSVLTQPPSASGTPGQRVTISCSGSSSNIQNNYVYWYQQLPGTAPKLLIYFNSQRPSGVPDRFSGSKSGTSASLAISGLQSEDEADYYCGTWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 61 - DNA, 1347 bp, synthetic construct
  source 1..1347: mol_type = other DNA
gaggttcagctgctggaatctggcggaggatctgttcagcctggcggctctctgagactgtcttgtaccgcctccggcttcaacttcgaggactacggcatgaactgggtccgacaggctcctggcaaaggcctggattgggtgtcctccatcaaccggaacggcgacatctccacctaccacgtggactccgtgaagggcagattcaccatcagccgggacaactccaagaacaccctgtacctgcagatgaactccctgagagccgaggacaccgccatctactactgcaccaagaaccacgactaccacggcatggatgtgtggggccaaggcacaacagtgaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 62 - DNA, 648 bp, synthetic construct
  source 1..648: mol_type = other DNA
cagtctgtgctgacccagcctccttctgcttctggcacacctggccagagagtgaccatctcctgttccggctctcggtccaacatcggcaacaacttcgtgaactggtatcagcagatccccggcaccgctcctaagctgctgatctacctgaactcccagcggccttctggcgtgcccgatagattctccggctctaagtctggcacctctgccagcctggctatctctggcctgcagtctgatgacgaggccgactactactgcgcctcctgggacgattctctgaacggctgggttttcggcggaggcaccaaactgacagtgctgggacagcctaaggcagccccctccgtgaccctgttcccgccatcatccgaagaactgcaggccaacaaggccacgctcgtgtgcctgatttccgacttctacccgggggccgtgactgtggcctggaaggcagactcaagccctgtgaaggctggcgtcgagactaccaccccgtcgaagcaatccaacaacaaatacgcggcgtccagctacctgagcctgacccctgagcagtggaaatcccaccggtcctactcgtgccaagtcacccacgagggatccactgtggaaaagaccgtggcgccgactgagtgttcc

SEQ ID NO: 63 - DNA, 1347 bp, synthetic construct
  source 1..1347: mol_type = other DNA
gaggttcagctgctggaatctggcggaggatctgttcagcctggcggctctctgagactgtcttgtaccgcctccggcttcaacttcgaggactacggcatgaactgggtccgacaggctcctggcaaaggcctggattgggtgtcctctatcaaccggcagggcgacatctccacctaccacgtggactctgtgaagggcagattcaccatcagccgggacaactccaagaacaccctgtacctgcagatgaactccctgagagccgaggacaccgccatctactactgcaccaagaaccacgactaccacggcatggatgtgtggggacaaggcacaacagtgaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 64 - DNA, 1338 bp, synthetic construct
  source 1..1338: mol_type = other DNA
gaggttcagctgctggaatctggcggaggattggttcagcctggcggctctctgagactgtcttgcgaggcctctggcttcatcttccgggactacaacatgaactgggtccgacaggctcctggcaaaggcctggaatgggtgtccatcatcaccaacgacggctcctccaccgcctactccgattctgtgaagggcagattcaccatcagccgggacaactccaagaactccctgtacctgcagatgaacagcctgagagccgaggacaccgccatctactactgcgtgaaggtgtactacggcatggacgattggggccagggcaccacagtgacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 65 - DNA, 648 bp, synthetic construct
  source 1..648: mol_type = other DNA
cagtctgtgctgacccagcctccttctgcttctggcacacctggccagagagtgaccatctcttgctctggcggctcctccaacatcggctccaattacgtgtcctggtatcagcagctgcccggcacagctcccaaactgctgatctacttcaacttccagcggccttctggcgtgcccgacagattctctggctctaagtctggcacctctgccagcctggctatctctggactgcagagcgaggacgaggccgactactattgtgccgcctgggacgacagagtgaacgtgtgggttttcggcggaggcaccaagctgacagttctgggacagcctaaggcagccccctccgtgaccctgttcccgccatcatccgaagaactgcaggccaacaaggccacgctcgtgtgcctgatttccgacttctacccgggggccgtgactgtggcctggaaggcagactcaagccctgtgaaggctggcgtcgagactaccaccccgtcgaagcaatccaacaacaaatacgcggcgtccagctacctgagcctgacccctgagcagtggaaatcccaccggtcctactcgtgccaagtcacccacgagggatccactgtggaaaagaccgtggcgccgactgagtgttcc

SEQ ID NO: 66 - DNA, 1344 bp, synthetic construct
  source 1..1344: mol_type = other DNA
gaagttcagcttttggagtccggtgggggattggttcagccagggggaagtctgcgattgtcatgtgcagcatcctccttcacattctataattatgacatgacatgggtacggcaggcaccaggaaaaggactggagtgggttgctgggattgggacatcaggaactgatatgtattacgcagattcagtaaagggtcgctttacaataagccgcgataatagtaaaaatacattgtatcttcaaatgaactcactcagagccgaggacactgctatttactactgcgctaaagattactattattacgggatggacgtatggggccttggcacaactgttaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 67 - DNA, 645 bp, synthetic construct
  source 1..645: mol_type = other DNA
cagagcgtcctcactcagcctccctcagctagtggcactcccggacagagggtaataatatcttgcagcggttcaaccagtaatataggtaataattacgtaagttggtatcaacagctcccaggcacagctcctaaattgctcatctactatgatcaacgcccttccggtgttcctgataggttttccggttcaaagagtggcacctccgccagtctcgctatatctggacttcaatcagaggacgaagctgactattattgtgccgcttgggacgacagcctgaacgggtgggttttcggcgggggcactaagctgacagtccttggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 68 - DNA, 1344 bp, synthetic construct
  source 1..1344: mol_type = other DNA
gaggttcagctgctggaatctggcggaggattggttcagcctggcggttctctgagactgtcttgtgccgcctcctccttcaccttctacaactacgacatgacctgggtccgacaggcccctggcaaaggattggaatgggtggccggcatcggcacctctggcaccgatatgtactacgccgactccgtgaagggcagattcaccatctctcgggacaactccaagaacaccctgtacctgcagatgaactccctgagagccgaggacaccgccatctactactgcgccaaggattactactactacggcatggacgtgtggggccaaggcacaacagtgacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 69 - DNA, 1347 bp, synthetic construct
  source 1..1347: mol_type = other DNA
gaggttcagctgctggaatctggcggaggattggttcagcctggcggctctctgagactgtcttgcgaggcctctggcttcgacttctccacctacgccatgaactgggtccgacaggctcctggcaaaggcctggaatgggtcgccaccatcaattccgagggcaccatcacctctcacgcccctgctgtgaagggcagattcaccatctctcgggacaactccaagaacaccgcctacgtgcagatgaactccctgagagccgaggactccgccgtgtactactgttcttctaccaccggcaccacacacggcatggatgtgtggggacgcggcaccacagtgaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 70 - DNA, 648 bp, synthetic construct
  source 1..648: mol_type = other DNA
cagtctgtgctgacccagcctccttctgcttctggcacacctggccagagagtgaccatctcttgctccggctcctccagcaacatccagaacaactacgtgtactggtatcagcagctgcccggcaccgctcctaaactgctgatctacttcaactcccagcggccttctggcgtgcccgatagattctccggctctaagtctggcacctctgccagcctggctatctctggactgcagagcgaggacgaggccgactactattgtggcacctgggacgattccctgaacggctgggttttcggcggaggcacaaagctgacagtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 71 - protein, 7 aa, synthetic construct
GFNFEDY

SEQ ID NO: 72 - protein, 7 aa, synthetic construct
NRQGDIS

SEQ ID NO: 73 - protein, 9 aa, synthetic construct
NHDYHGMDV

SEQ ID NO: 74 - protein, 13 aa, synthetic construct
SGSRSNIGNNFVN

SEQ ID NO: 75 - protein, 7 aa, synthetic construct
LNSQRPS

SEQ ID NO: 76 - protein, 11 aa, synthetic construct
ASWDDSLNGWV

SEQ ID NO: 77 - protein, 7 aa, synthetic construct
GFIFRDY

SEQ ID NO: 78 - protein, 6 aa, synthetic construct
TNDGSS

SEQ ID NO: 79 - protein, 7 aa, synthetic construct
VYYGMDD

SEQ ID NO: 80 - protein, 13 aa, synthetic construct
SGGSSNIGSNYVS

SEQ ID NO: 81 - protein, 7 aa, synthetic construct
FNFQRPS

SEQ ID NO: 82 - protein, 11 aa, synthetic construct
AAWDDRVNVWV

SEQ ID NO: 83 - protein, 7 aa, synthetic construct
SFTFYNY

SEQ ID NO: 84 - protein, 6 aa, synthetic construct
GTSGTD

SEQ ID NO: 85 - protein, 9 aa, synthetic construct
DYYYYGMDV

SEQ ID NO: 86 - protein, 13 aa, synthetic construct
SGSTSNIGNNYVS

SEQ ID NO: 87 - protein, 6 aa, synthetic construct
YDQRPS

SEQ ID NO: 88 - protein, 11 aa, synthetic construct
AAWDDSLNGWV

SEQ ID NO: 89 - protein, 7 aa, synthetic construct
GFDFSTY

SEQ ID NO: 90 - protein, 6 aa, synthetic construct
NSEGTI

SEQ ID NO: 91 - protein, 10 aa, synthetic construct
TTGTTHGMDV

SEQ ID NO: 92 - protein, 13 aa, synthetic construct
SGSSSNIQNNYVY

SEQ ID NO: 93 - protein, 7 aa, synthetic construct
FNSQRPS

SEQ ID NO: 94 - protein, 11 aa, synthetic construct
GTWDDSLNGWV

SEQ ID NO: 95 - protein, 10 aa, synthetic construct
GFNFEDYGMN

SEQ ID NO: 96 - protein, 11 aa, synthetic construct
SINRQGDISTY

SEQ ID NO: 97 - protein, 9 aa, synthetic construct
NHDYHGMDV

SEQ ID NO: 98 - protein, 13 aa, synthetic construct
SGSRSNIGNNFVN

SEQ ID NO: 99 - protein, 7 aa, synthetic construct
LNSQRPS

SEQ ID NO: 100 - protein, 11 aa, synthetic construct
ASWDDSLNGWV

SEQ ID NO: 101 - protein, 10 aa, synthetic construct
GFIFRDYNMN

SEQ ID NO: 102 - protein, 10 aa, synthetic construct
IITNDGSSTA

SEQ ID NO: 103 - protein, 7 aa, synthetic construct
VYYGMDD

SEQ ID NO: 104 - protein, 13 aa, synthetic construct
SGGSSNIGSNYVS

SEQ ID NO: 105 - protein, 7 aa, synthetic construct
FNFQRPS

SEQ ID NO: 106 - protein, 11 aa, synthetic construct
AAWDDRVNVWV

SEQ ID NO: 107 - protein, 10 aa, synthetic construct
SFTFYNYDMT

SEQ ID NO: 108 - protein, 10 aa, synthetic construct
GIGTSGTDMY

SEQ ID NO: 109 - protein, 9 aa, synthetic construct
DYYYYGMDV

SEQ ID NO: 110 - protein, 13 aa, synthetic construct
SGSTSNIGNNYVS

SEQ ID NO: 111 - protein, 6 aa, synthetic construct
YDQRPS

SEQ ID NO: 112 - protein, 11 aa, synthetic construct
AAWDDSLNGWV

SEQ ID NO: 113 - protein, 10 aa, synthetic construct
GFDFSTYAMN

SEQ ID NO: 114 - protein, 10 aa, synthetic construct
TINSEGTITS

SEQ ID NO: 115 - protein, 10 aa, synthetic construct
TTGTTHGMDV

SEQ ID NO: 116 - protein, 13 aa, synthetic construct
SGSSSNIQNNYVY

SEQ ID NO: 117 - protein, 7 aa, synthetic construct
FNSQRPS

SEQ ID NO: 118 - protein, 11 aa, synthetic construct
GTWDDSLNGWV

SEQ ID NO: 119 - protein, 8 aa, synthetic construct
GFNFEDYG

SEQ ID NO: 120 - protein, 9 aa, synthetic construct
INRQGDIST

SEQ ID NO: 121 - protein, 11 aa, synthetic construct
TKNHDYHGMDV

SEQ ID NO: 122 - protein, 8 aa, synthetic construct
RSNIGNNF

SEQ ID NO: 123 - skipped

SEQ ID NO: 124 - protein, 11 aa, synthetic construct
ASWDDSLNGWV

SEQ ID NO: 125 - protein, 8 aa, synthetic construct
GFIFRDYN

SEQ ID NO: 126 - protein, 8 aa, synthetic construct
ITNDGSST

SEQ ID NO: 127 - protein, 9 aa, synthetic construct
VKVYYGMDD

SEQ ID NO: 128 - protein, 8 aa, synthetic construct
SSNIGSNY

SEQ ID NO: 129 - skipped

SEQ ID NO: 130 - protein, 11 aa, synthetic construct
AAWDDRVNVWV

SEQ ID NO: 131 - protein, 8 aa, synthetic construct
SFTFYNYD

SEQ ID NO: 132 - protein, 8 aa, synthetic construct
IGTSGTDM

SEQ ID NO: 133 - protein, 11 aa, synthetic construct
AKDYYYYGMDV

SEQ ID NO: 134 - protein, 8 aa, synthetic construct
TSNIGNNY

SEQ ID NO: 135 - skipped

SEQ ID NO: 136 - protein, 11 aa, synthetic construct
AAWDDSLNGWV

SEQ ID NO: 137 - protein, 8 aa, synthetic construct
GFDFSTYA

SEQ ID NO: 138 - protein, 8 aa, synthetic construct
INSEGTIT

SEQ ID NO: 139 - protein, 12 aa, synthetic construct
SSTTGTTHGMDV

SEQ ID NO: 140 - protein, 8 aa, synthetic construct
SSNIQNNY

SEQ ID NO: 141 - skipped

SEQ ID NO: 142 - protein, 11 aa, synthetic construct
GTWDDSLNGWV

SEQ ID NO: 143 - protein, 6 aa, Homo sapiens
FPLDTL

SEQ ID NO: 144 - protein, 4 aa, Homo sapiens
EIGL

SEQ ID NO: 145 - protein, 4 aa, synthetic construct
YGMN

SEQ ID NO: 146 - protein, 9 aa, synthetic construct
RQAPGKGLD

SEQ ID NO: 147 - protein, 6 aa, synthetic construct
SSINRN

SEQ ID NO: 148 - protein, 5 aa, synthetic construct
GDIST

SEQ ID NO: 149 - protein, 9 aa, synthetic construct
FTISRDNSK

SEQ ID NO: 150 - protein, 8 aa, synthetic construct
CTKNHDYH

SEQ ID NO: 151 - protein, 32 aa, synthetic construct
QSVLTQPPSASGTPGQRVTISCSGSRSNIGNN

SEQ ID NO: 152 - protein, 24 aa, synthetic construct
LNSQRPSGVPDRFSGSKSGTSASL

SEQ ID NO: 153 - protein, 5 aa, synthetic construct
RDYNM

SEQ ID NO: 154 - protein, 8 aa, synthetic construct
QAPGKGEW

SEQ ID NO: 155 - protein, 20 aa, synthetic construct
SIITNDGSSTAYSDSVKGRF

SEQ ID NO: 156 - protein, 34 aa, synthetic construct
QSVLTQPPSASGTPGQRVTISCSGGSSNIGSNYV

SEQ ID NO: 157 - skipped

SEQ ID NO: 158 - protein, 21 aa, synthetic construct
QRPSGVPDRFSGSKSGTSASL

SEQ ID NO: 159 - protein, 5 aa, synthetic construct
DRVNV

SEQ ID NO: 160 - protein, 24 aa, synthetic construct
YYDQRPSGVPDRFSGSKSGTSASL

SEQ ID NO: 161 - protein, 12 aa, synthetic construct
SGGGLVQPGGSL

SEQ ID NO: 162 - protein, 6 aa, synthetic construct
EASGFD

SEQ ID NO: 163 - protein, 9 aa, synthetic construct
RQAPGKGLE

SEQ ID NO: 164 - protein, 19 aa, synthetic construct
INSEGTITSHAPAVKGRFT

SEQ ID NO: 165 - protein, 15 aa, synthetic construct
CSSTTGTTHGMDVWG

SEQ ID NO: 166 - protein, 34 aa, synthetic construct
QSVLTQPPSASGTPGQRVTISCSGSSSNIQNNYV

SEQ ID NO: 167 - protein, 23 aa, synthetic construct
NSQRPSGVPDRFSGSKSGTSASL

SEQ ID NO: 168 - protein, 6 aa, synthetic construct
SLNGWV

SEQ ID NO: 169 - skipped

SEQ ID NO: 170 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 171 - protein, 64 aa, Homo sapiens
GRELVIPCRVTSPNITVTLKKFPLDTLIPDGKRIIWDSRKGFIISNATYKEIGLLTCEATVNGH

SEQ ID NO: 172 - protein, 98 aa, Homo sapiens
IDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVH

SEQ ID NO: 173 - protein, 162 aa, synthetic construct
GRELVIPCRVTSPNITVTLKKFPLDTLIPDGKRIIWDSRKGFIISNATYKEIGLLTCEATVNGHIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVH

SEQ ID NO: 174 - protein, 8 aa, synthetic construct
LEVLFQGP

SEQ ID NO: 175 - protein, 18 aa, synthetic construct
SINRNGDISTYHVDSVKG

SEQ ID NO: 176 - protein, 7 aa, synthetic construct
NRNGDIS

SEQ ID NO: 177 - protein, 11 aa, synthetic construct
SINRNGDISTY

SEQ ID NO: 178 - protein, 9 aa, synthetic construct
INRNGDIST

SEQ ID NO: 179 - protein, 5 aa, synthetic construct
GGGGS

SEQ ID NO: 180 - protein, 6 aa, synthetic construct
HHHHHH

SEQ ID NO: 181 - protein, 202 aa, Homo sapiens
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDK

SEQ ID NO: 182 - protein, 202 aa, Macaca fascicularis
DTGRPFVEMYSEIPEIIHMTEGRELIIPCRVTSPNITVTLKKFPLDTLIPDGKRVIWDSRKGFIISNATYKEIGLLTCEATVNGHLYKTNYLTHRQTNTIIDVQISTPRPVKLLRGHTLILNCTATTPLNTRVQMTWSYPDEKNKRASVRRRIDQSNSHANIFYSVLTIDKVQNKDKGLYTCRVRSGPSFKSVNTSVHIYDK

SEQ ID NO: 183 - protein, 202 aa, Rattus sp.
DAGSPFIEMHSDIPKLVHMTEGRELIIPCRVTSPNITVTLKKFPFDALTPDGQRIAWDSRRGFIIANATYKEIGLLTCEATVNGHLYQTSYLTHRQTNTILDVQISPPSPVRFLRGQTLVLNCTVTTDLNTRVQMSWNYPGKATKRASIRQRIDQSNPHSNVFHSVLKINNVESRDKGLYTCRVKSGSSFRTFNTSVHVYEK

SEQ ID NO: 184 - protein, 202 aa, Mus sp.
DAGSPFIEMHTDIPKLVHMTEGRQLIIPCRVTSPNVTVTLKKFPFDTLTPDGQRITWDSRRGFIIANATYKEIGLLNCEATVNGHLYQTNYLTHRQTNTILDVQIRPPSPVRLLHGQTLVLNCTATTELNTRVQMSWNYPGKATKRASIRQRIDRSHSHNNVFHSVLKINNVESRDKGLYTCRVKSGSSFQSFNTSVHVYEK

SEQ ID NO: 185 - protein, 202 aa, Oryctolagus sp.
DADKPFVEMYTEIPEVLYMHEGRELVIPCRVTSPNINVTLKQFPSDQVIPHGERITWNSRRGFIVSSATYKEIGILYCEASVNGQVYKTHYLIHRLTNTILDVQMSTPSPVKLLKGRALNLNCTATTPLNTRVQMTWSYPGQTSKRASVRQRIDQSSAHANVFYSVLVIDQVQNRDKGLYTCQVKSGPSFRSASTSVHVYDK

SEQ ID NO: 186 - protein, 202 aa, Gallus sp.
DTSNPFVEMHSDIPKIIHMTVGKEMIIPCRVTAPNIAVTLKKIPRETLIPDGKTIIWDNMRGFRIPEATYRFIGLLSCETTIGGHKYSTKYLTHRETNTIFDIKLSTPRLVKLLKGDSLAINCTVKAAWNTRVQMTWTYPGEAMKRGSVTQRIDQKNREANVFYSILVIDKVRDIDKGQYACHVKSGPSNKLVNTTVIVYDK